SEQUENCE LISTING (66 sequences)

SEQ ID NO: 1 - DNA, 20 bp, Artificial Sequence
  Primer
gaaggatgtggtccgagtgt

SEQ ID NO: 2 - DNA, 20 bp, Artificial Sequence
  Primer
gtgaagtgagggctcccata

SEQ ID NO: 3 - DNA, 20 bp, Artificial Sequence
  Primer
aaccccaagatgcacaactc

SEQ ID NO: 4 - DNA, 20 bp, Artificial Sequence
  Primer
gcttagcctcgtcgatgaac

SEQ ID NO: 5 - DNA, 23 bp, Artificial Sequence
  Primer
aatacctcagcctccagcagatg

SEQ ID NO: 6 - DNA, 24 bp, Artificial Sequence
  Primer
tgcgtcacaccattgctattcttc

SEQ ID NO: 7 - DNA, 21 bp, Artificial Sequence
  Primer
gtggatgtctttgtgcaccag

SEQ ID NO: 8 - DNA, 20 bp, Artificial Sequence
  Primer
gacacggatggattccagac

SEQ ID NO: 9 - DNA, 22 bp, Artificial Sequence
  Primer
ggacctggactttattctctcc

SEQ ID NO: 10 - DNA, 20 bp, Artificial Sequence
  Primer
gataggtgaagctgcaggtg

SEQ ID NO: 11 - DNA, 20 bp, Artificial Sequence
  Primer
ccttccaatcagctgccttc

SEQ ID NO: 12 - DNA, 20 bp, Artificial Sequence
  Primer
gatctgcctgcctctctcat

SEQ ID NO: 13 - DNA, 20 bp, Artificial Sequence
  Primer
gtcgacatcacttgccagac

SEQ ID NO: 14 - DNA, 20 bp, Artificial Sequence
  Primer
tggtcggatgaaactcctcc

SEQ ID NO: 15 - DNA, 20 bp, Artificial Sequence
  Primer
aggagtgtgtgctaaccgtt

SEQ ID NO: 16 - DNA, 20 bp, Artificial Sequence
  Primer
cagttcgtttcagtgccaca

SEQ ID NO: 17 - DNA, 20 bp, Artificial Sequence
  Primer
gagagactgggttattcctc

SEQ ID NO: 18 - DNA, 21 bp, Artificial Sequence
  Primer
gatgctgtagaaaaccttgcc

SEQ ID NO: 19 - DNA, 20 bp, Artificial Sequence
  Primer
tggacacgtctgtgctcttc

SEQ ID NO: 20 - DNA, 20 bp, Artificial Sequence
  Primer
gtcttggcgtcttctcgaac

SEQ ID NO: 21 - DNA, 20 bp, Artificial Sequence
  Primer
atttgtgggaccctcgacat

SEQ ID NO: 22 - DNA, 20 bp, Artificial Sequence
  Primer
ggtaaaagctcgcccacaaa

SEQ ID NO: 23 - DNA, 23 bp, Artificial Sequence
  Primer
ctgactacctcatgaagatcctc

SEQ ID NO: 24 - DNA, 22 bp, Artificial Sequence
  Primer
cattgccaatggtgatgacctg

SEQ ID NO: 25 - DNA, 20 bp, Artificial Sequence
  Primer
agagggacatctcacggttc

SEQ ID NO: 26 - DNA, 19 bp, Artificial Sequence
  Primer
ggttgccccagaagtatcg

SEQ ID NO: 27 - DNA, 21 bp, Artificial Sequence
  Primer
ggtgccagttaaagatgacgc

SEQ ID NO: 28 - DNA, 20 bp, Artificial Sequence
  Primer
gaggcaaaatggtcggcaag

SEQ ID NO: 29 - DNA, 21 bp, Artificial Sequence
  Primer
gacctccacagagaagtcgag

SEQ ID NO: 30 - DNA, 21 bp, Artificial Sequence
  Primer
tgcctttttcttagggcagag

SEQ ID NO: 31 - DNA, 19 bp, Artificial Sequence
  Primer
gcaagggcaagctctatgg

SEQ ID NO: 32 - DNA, 22 bp, Artificial Sequence
  Primer
aggactcgtaggcgttgtagtt

SEQ ID NO: 33 - DNA, 18 bp, Artificial Sequence
  Primer
cgctttctcccagaccaa

SEQ ID NO: 34 - DNA, 24 bp, Artificial Sequence
  Primer
ggcagacaggttaaagtagaggag

SEQ ID NO: 35 - DNA, 18 bp, Artificial Sequence
  Primer
caccagcagcgactctga

SEQ ID NO: 36 - DNA, 22 bp, Artificial Sequence
  Primer
gatccagactctgaccttttgc

SEQ ID NO: 37 - DNA, 20 bp, Artificial Sequence
  Primer
agatgcctcacacggagact

SEQ ID NO: 38 - DNA, 20 bp, Artificial Sequence
  Primer
ggactggtggaagaatcagg

SEQ ID NO: 39 - DNA, 18 bp, Artificial Sequence
  Primer
cgaccatctgccgctttg

SEQ ID NO: 40 - DNA, 21 bp, Artificial Sequence
  Primer
gccgcagcttacacatgttct

SEQ ID NO: 41 - DNA, 20 bp, Artificial Sequence
  Primer
agcacatcaactcggaggag

SEQ ID NO: 42 - DNA, 20 bp, Artificial Sequence
  Primer
agcacatcaactcggaggag

SEQ ID NO: 43 - DNA, 21 bp, Artificial Sequence
  Primer
acagcaaatgacagctgcaaa

SEQ ID NO: 44 - DNA, 17 bp, Artificial Sequence
  Primer
tcggcatcgcggttttt

SEQ ID NO: 45 - DNA, 20 bp, Artificial Sequence
  Primer
ggttggtatccggggacttc

SEQ ID NO: 46 - DNA, 20 bp, Artificial Sequence
  Primer
tccgttggaactgatggagt

SEQ ID NO: 47 - DNA, 18 bp, Artificial Sequence
  Primer
accaggccatggatgaag

SEQ ID NO: 48 - DNA, 20 bp, Artificial Sequence
  Primer
cttaattgctggggaattgg

SEQ ID NO: 49 - DNA, 20 bp, Artificial Sequence
  Primer
ctggctgtggcaaggtcttc

SEQ ID NO: 50 - DNA, 20 bp, Artificial Sequence
  Primer
cagccctcaaactcgcactt

SEQ ID NO: 51 - DNA, 20 bp, Artificial Sequence
  Primer
ggaagcccaagaacctgaat

SEQ ID NO: 52 - DNA, 19 bp, Artificial Sequence
  Primer
gttgctggagttgctggaa

SEQ ID NO: 53 - DNA, 21 bp, Artificial Sequence
  Primer
aatacttcccccacaacacaa

SEQ ID NO: 54 - DNA, 18 bp, Artificial Sequence
  Primer
ctctcccgcaccagtcat

SEQ ID NO: 55 - DNA, 18 bp, Artificial Sequence
  Primer
acgccgagttgagcaaga

SEQ ID NO: 56 - DNA, 18 bp, Artificial Sequence
  Primer
tctgcctcctccacgaag

SEQ ID NO: 57 - DNA, 19 bp, Artificial Sequence
  Primer
accgccaccaaactgagat

SEQ ID NO: 58 - DNA, 20 bp, Artificial Sequence
  Primer
ttgtagtgggcagtgggatt

SEQ ID NO: 59 - DNA, 19 bp, Artificial Sequence
  Primer
aactcaagaaggcggatgg

SEQ ID NO: 60 - DNA, 19 bp, Artificial Sequence
  Primer
ggaggaaaaccttcgtgct

SEQ ID NO: 61 - DNA, 20 bp, Artificial Sequence
  Primer
acccagttcatagcggtgac

SEQ ID NO: 62 - DNA, 20 bp, Artificial Sequence
  Primer
ccattgggagtacccaggtt

SEQ ID NO: 63 - DNA, 19 bp, Artificial Sequence
  Primer
agccacatcgctcagacac

SEQ ID NO: 64 - DNA, 19 bp, Artificial Sequence
  Primer
gcccaatacgaccaaatcc

SEQ ID NO: 65 - DNA, 27 bp, Artificial Sequence
  Primer
ggatgttattaagatgaagatagttgg

SEQ ID NO: 66 - DNA, 25 bp, Artificial Sequence
  Primer
cctaaactccccttcaaaatctatt

(19) United States
(12) Patent Application Publication
(10) Pub. No.: US 2020/0062719 A1
(43) Pub. Date: Feb. 27, 2020

(54) Application of Imidazopyridine Derivatives in Regenerative Medicine

(71) Applicants: University of Heidelberg, Heidelberg (DE); Universitätsklinikum Jena, Jena (DE)

(72) Inventors: Xinlai Cheng, Heddesheim (DE); Stefan Wolfl, Dossenheim (DE); Ralf Mrowka, Jena (DE); James Adjaye, Düsseldorf (DE)

(73) Assignees: University of Heidelberg, Heidelberg (DE); Universitätsklinikum Jena, Jena (DE)

(21) Appl. No.: 16/540,100

(22) Filed: Aug. 14, 2019

(30) Foreign Application Priority Data
Aug. 14, 2018 (EP) 18188930.4

(51) Int. Cl. C07D 247/02; C12N 15/113; C12N 5/074

(52) U.S. Cl. CPC C07D 247/02; C12N 15/113; C12N 5/0696; C12N 2501/603; C12N 2310/141; C12N 2501/602; C12N 2501/606; C12N 2501/608; C12N 2501/604

(57) ABSTRACT

A method of producing a pluripotent stem cell is provided. The method is comprising contacting a non-pluripotent donor cell obtained from a mammalian donor with a compound characterized by general formulas (1) and (3). Furthermore, methods for inducing OCT4 and NANOG, increasing histone 3 lysine methylation and the maintenance of pluripotency are provided.

### CROSS-REFERENCE TO RELATED APPLICATIONS

Benefit is claimed to European Patent Application No. EP18188930.4, filed Aug. 14, 2018, the contents of which are incorporated by referenced herein in their entirety.

### FIELD

The present invention relates to an in vitro/ex vivo method for producing induced pluripotent stem cells from non-pluripotent donor cells.

### BACKGROUND

Pluripotent stem cells, such as embryonic stem cells, have the potential to differentiate into any of the three germ layers: endoderm, mesoderm and ectoderm, and therefore give rise to any cell type derived from these germ layers.

Nuclear reprogramming from terminally differentiated somatic cells into induced pluripotent stem cells (iPSCs) achieved by ectopic expression of a core of transcription factors, like Oct4, Sox2, Klf4, c-Myc, Nanog and Lin28 provides a promising source for autologous organ transplantation, drug discovery and disease modelling, opening a new era in the field of regenerative medicine.

Methods available for the generation of pluripotent stem cells are somatic cell nuclear transfer (SCNT) and reprogramming of human fibroblasts. Both methods have their distinct disadvantages and limitations. The reprogramming of human fibroblasts is a time-consuming (3-4 weeks) and inefficient (<0.01% of cells) process. It also can be hampered by random mutations appearing in the course of reprogramming and during maintenance. Therefore, higher reprogramming efficiency and optimized cultivation conditions for iPSC maintenance can contribute to risk reduction. Improved reprogramming efficiency for generation of individual patients own iPSCs will also allow to avoid risks associated with autologous iPSC cell line derived allografts, like immunoreaction, evidenced by allogeneic transplantation of iPSC-derived neural cells in nonhuman primates (Morizane et al. Stem Cell Reports. 2013 Sep. 26; 1(4):283-9).

In contrast to using genetic manipulation for reprogramming somatic cells, the use of small molecules shows distinct advantages in controlling the reprogramming process. Recently a number of small molecules have been disclosed that greatly contribute to the establishment of more efficient and reliable protocols for the generation and maintenance of iPSCs (Watanabe et al, Nat Biotechnol. 2007 June; 25(6):681-6; Ying et al Nature. 2008 May 22; 453(7194):519-23). Small molecule cocktails that exhibit excellent reprogramming efficiency for the generation of mouse iPSCs have been disclosed recently (Hou et al, Science. 2013 Aug. 9; 341(6146):651-4; Zhao et al, Cell. 2015 Dec. 17; 163(7):1678-91).

### SUMMARY

Herein the inventors disclose that 2-arylpyrimidazoles—including zolpidem, an approved drug for insomnia treatment, —act as novel potent pluripotency inducers for the generation and maintenance of human iPSCs.

Based on the above-mentioned state of the art, the objective of the present invention is to provide means and methods for the induction of pluripotency in mammalian donor cells and the maintenance of pluripotency in these cells. This objective is attained by the subject matter of the claims of the present specification.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D show the validation of activity of O4I3 (101) in luciferase assays. A) Synthesis of O4I3 (101). B) Relative luciferase activity in NCCIT-Oct4 cells treated with increasing concentrations of O4I3 for 48 h. The data were collected from at least five independent experiments. C) Relative luciferase activity in NCCIT-Nanog cells. Data are represented as mean±SD from at least 20 independent experiments ***: p<0.001; **: p<0.01; *: p<0.05. D) Continuous activation of Oct4 in NCCIT-Oct4 reporter cells at indicated days (D1, D2, D3 and D4). ***: p<0.001; **: p<0.01; *: p<0.05.

FIGS. 2A-2D show that O4I3 supports maintenance of human induced pluripotent stem cells (hiPSCs). A) O4I3 promoted the viability of hiPSCs in single cell expansion. B) Homogeneous expression of Tra-1-80 and Oct4 up to passage (p) 30 in hiPSCs treated with 50 nM O4I3 analyzed by FACS C) Relative fold changes to non-treatment in hiPSCs treated with O4I3 (50 nM) for 24 h analyzed by qRT-PCR. D) Gene expression profiling analysis of functional annotation enrichment for categories related to pluripotency, development and homeostasis in O4I3-treated iPSCs for 24 h. Data are represented as mean±SD from at least 3 independent experiments ***: p<0.001; **: p<0.01; *: p<0.05.

FIGS. 3A-3C show that O4I3 facilitates the conversion of fibroblasts into hiPSCs. A) Induction of Oct4 and B) Nanog expression in human fibroblasts (HF2) treated with O4I3 (0.5 μM) at day 1 (1 D), day 3 (3D), day 5 (5D) and day 7 (7D) detected by qRT-PCR. C) O4I3 increased the formation of AP+ colonies in episome-based reprogramming of four human fibroblasts.

FIGS. 4A-4F show cell identity of newly generated hiPSCs. A) Established hiPSC cell lines showing hESC-like morphologies. B) Expression of differentiation markers of three germ layers detected by qRT-PCR. C) Comparison of E-Cadherin and Tra-1-60 expression by FACS in HF2 and iPSCs. D) pluripotency-associated gene expression by immunoblotting. NCCIT and PSC1/2/3 were used as positive control. E) Pairwise comparison of log 2-transformed gene expression using scatter plots. Red dashed lines depict the two-fold change in expression values. F) Heatmap of expression profiles of selected pluripotency-associated genes (bright green label) and fibroblast-associated genes (dark green label) expression. The cells in both heatmaps depict log 2 transformed gene expression values, centred on the gene-specific median expression across all samples. Genes and samples are reordered using hierarchical clustering based on complete linkage and Euclidean distance.

FIGS. 5A-5E show that O4I3 facilitates the binding of H3K4Me3 to promoter of Oct4 in human fibroblasts. A) Venn diagram for comparison of up- and down-regulated gene expression in O4I3 (500 nM) treated human fibroblasts (HFF) with ectopic expression of Oct4, Sox2, Klf4 and Myc (OSKM) as well as hiPSCs. HFF treated with O4I3; HFF transfected with episomal OSKM; hiPSCs. B) Induction of H3K4Me4 by O4I3 in HFFs detected by immunoblotting. C) Inhibitors and D) activators of reprogramming, E) Enrichment of H3K4Me3 at the promoter of Oct4 analyzed by Chip-qPCR. The data were normalized to iPSCs.

FIGS. 6A-6B show activity in NCCIT-Oct4 reporter cells treated with derivatives. A) Activity of compounds 100 to 123 relative to treatment with compound 101 (O4I3). B) Activity of compounds 100 to 123 relative to control treatment (DMSO). Data are represented as mean±SD from at least 4 independent experiments.

FIG. 7 shows the maintenance of neuronal stem cells. Neural stem cells were cultivated in SPN medium (DMEM-F12 Knockout medium containing 2% StemPro® NSC SFM, EGF (20 ng/mL) and FGF (20 ng/mL)) until cell confluence reached 80-90%. Acutase was used to get single cell suspension and washed with PBS. 20,000 cells/cm⁻¹ were seeded into matrigel-coated plates using SPN medium with or without O4I3. The MTT assay was applied to determine cell viability.

FIG. 8 shows the luciferase activity in NCCIT-Oct4 cells (luciferase activity is driven by the Oct4-promoter) treated with increasing concentrations of zolpidem (Zo) for 48 hours.

FIG. 9 shows the luciferase activity in NCCIT-Nanog cells (luciferase activity is driven by the Nanog-promoter) treated with increasing concentrations of zolpidem (Zo) for 48 hours.

FIG. 10 shows the microarray analysis on the phosphorylation level of reprogramming-related proteins in NCCIT or JoPaca-1 cells. Cells were analysed in the presence (24 hours O4I3 1 μM) or absence (NT) of O4I3.

FIG. 11 shows induction of differentiation markers in hPSCs treated with O4I3 at the higher concentration (10 μM).

FIG. 12 shows comparative qRT-PCR analysis of reprogramming related gene expression in pluripotent stem cells (PSC), human fibroblasts (HF2) and human fibroblasts treated with O4I3 (0.5 μM) at day 1 (O4I3 1 D), day 3 (O4I3 3D), day 5 (O4I3 5D) and day 7 (O4I3 7D).

FIG. 13 shows that O4I3 (50 nM) increases the formation of alkaline phosphatase positive (AP+) colonies in episome-based reprogramming of human fibroblasts (HF 1 and HF2). 1 and 2 AP+ colonies were obtained by using episome-based reprogramming in HF5 in the presence of Zolpidem (10 μM) from two independent experiments.

FIG. 14 shows the abundance of H3K4Me3 in O4I3-treated fibroblasts in the presence/absence of reprogramming factors Oct4, Sox2, Klf4 and Myc (OSKM). The fluorescent intensities of single cells selected by using ImageJ software are compared.

FIG. 15 shows functional enrichment analysis of differentially expressed genes in O4I3-treated HFF, including only the top ten Gene Ontology terms enriched in the sets of up-regulated and down-regulated genes, separately. The most enriched terms in the sets of common genes that are also differentially expressed in hPSC (as compared to HFF) was included.

FIG. 16 shows the activation of H3K4Me3. Levels of H3K4Me3 have been determined after indicated treatment and normalized to the level of iPSCs.

FIGS. 17A-17F O4I3 Is an Epigenetic Modulator. (A) Venn diagram of up- and downregulated genes in O4I3 (50 nM)-treated human fibroblasts (HF) with ectopic expression of OCT4, SOX2, KLF4, and MYC (OSKM) as well as hiPSCs. HF-O4I3, HF cells treated with O4I3 (50 nM for 24 h); HF-OSKM, HF cells transfected with episomal OSKM; iPSCs, the newly generated iPSCs. Differential expression was calculated with respect to HF cells. (B) Comparison of expression profiles of genes involved in epigenetic modification, chromatin remodeling, and related biological processes. (C) Occupation of H3K4Me3 or H3K27Me3 at the promoter of OCT4 in HF cells transfected with OSKM alone or in the presence of 50 nM of O4I3 (03-OSKM) for 3 days. (D) O4I3 elevates the global levels of H3K4Me3 detected by immunocytochemistry. Cells were treated with 250 nM O4I3 for 3 days. Scale bar, 40 mm. (E) Immunoblotting results show that O4I3 promotes global H3K4Me3 expression, whereas it represses H3K9Me3 protein levels in a concentration- and time-dependent manner. Cells were treated either with increasing concentrations of O4I3 for 24 h or with 250 nM of O4I3 for the indicated time points. O4I3 protected methylated H3K4 with an IC50 value of 20 nM (F).

FIGS. 18A-18I O4I3 Is a Selective KDM5A Inhibitor. (A) Comparison of O4I3 inhibitory effect on KDM5, LSD1, and KDM4 in vitro using the whole-cell nuclear extraction. (B) The inhibitory effect of O4I3 on the members of KDM5 family of demethylases isolated from cells. (C) A selective KDM5A inhibitor JIB-04 induces OCT4 expression inNCCIT-OCT4 cells. Four histone demethylase inhibitors (HDMs), namely, CPI-455, JIB-04, GSK-J4, and daminozide, were incubated with NCCIT-OCT4 reporter cells for 48 h. (D) JIB-04 (5 mM) induces OCT4 expression in fibroblasts at the indicated time points (D, days). (E) Comparison of KDM5A and KDM5B expression levels in fibroblast (HF1), resistant fibroblast (HF4), HF4 transfected with OSKM, iPSCs, and NCCIT. (F) Knockdown of KDM5A (si5A) activates OCT4 in NCCIT-OCT4 reporter cells. Cells were transfected with various KDM5 siRNA concentrations for 48 h. (G) Transient knockdown (48 h) of KDM5A induces OCT4 and SOX2 expression in fibroblasts. (H) Overexpression of KDM5A (OE5A) compromises the effect of 0413 (100 nM, 48 h) in NCCIT-OCT4 and HEK-OCT4 cells. (I) The number of TRA-1-60-positive colonies was reduced in reprogramming-resistant fibroblasts with overexpression of KDM5A. In (C), (D), and (F) statistical significance was compared withmock (0.1% DMSO) treatment, in (G) with non-targeting siRNA and in (E) with HF4, whereas in (H) and (I) it was compared with OSKM-treated fibroblasts using two-way ANOVA followed by a post-hoc Tukey test. Data are represented as mean G SD. ***p<0.001, **p<0.01, *p<0.05.

FIGS. 19A-19F KDM5A Is a Reprogramming Barrier (A and B) (A) Suppression of KDM5A activity by siRNA or JIB-04 (5 mM, 48 h) promotes the enrichment of H3K4Me3 at the promoter of OCT4 in resistant fibroblasts, (B) but not that of H3K27Me3. Ct values were obtained from qRT-PCR. (C) Suppression of KDM5A activity by JIB-04 or KDM5A siRNA (siKA) induces POU5F1, SOX2, and CDH1mRNA levels during reprogramming, as determined by qRT-PCR. (D) Correlation between reprogramming efficiency and KDM5A expression in HF1-HF11. The same amount of each cDNA was used as template for qRT-PCR where the Ct values indicate the expression of KDM5A. (E) Knockdown effects of increasing concentrations of anti-KDM5A siRNA (siKDM5A, 48 h) on H3K4Me3 expression levels in HF. (F) Reprogramming efficiency using various patient primary fibroblasts, as determined by the number of TRA-1-60-positive colonies. Cells were either transiently transfected with the indicated siRNA oligos in the first 5 days or treated with JIB-04 (5 mM, 48 h). siK5ABCD, anti-KDM5A/B/C/D siRNA; siK5AD, anti-KDM5A/D siRNA; siK5CD, anti-KDM5C/D siRNA; siK5A, anti-KDM5A siRNA; siK5B, anti-KDM5B siRNA; siK5C, anti-KDM5C siRNA; and siK5D, anti-KDM5D siRNA. In (A), (B), and (F) statistical significance was compared with OSKM-treated fibroblasts, whereas in (C) it was compared with DMSO (0.1%) treatment using two-way ANOVA and a post-hoc Tukey test. Data are represented as mean G SD. ***p<0.001, **p<0.01, *p<0.05.

FIGS. 20A-20F Metabolic stability is the key factor for maintaining the activity of O4I2 derivatives in PSCs. A) Synthesis of O4I2 derivatives. B) Structures of carboxylic derivatives. C) Relative luciferase activity in HEK-OCT4, NCCIT-OCT4 and NCCIT-NANOG reporter cell lines upon treatment with O4I2 ester derivatives. 0.2% DMSO was used as mock treatment. D) Metabolic analysis of O4I2 incubated with NCCIT for 24 hours. O4I2 and its metabolite O4I2-COON were detected by HPLC (UV 254 nm, blue chromatogram) coupled to MS (Total ion chromatogram [TIC] indicated in black). Extracted ion chromatograms (EIC) of ions at m/z 255 (red, O4I2-COON) and m/z 283 (green, O4I2) are gated by their respective red and green boxes. E) Structures of non-carboxylic O4I2 derivatives. F) Activity of compounds (15-23) measured in the three reporter cell lines. Data are represented as mean values ±one SD from at least 10 independent experiments. *: p<0.05; **: p<0.01 and ***: p<0.001.

FIG. 21A-21E Reprogramming of human fibroblasts without exogenous overexpression of OCT4. A) Illustration of hiPSCs generation without OCT4. HF1-LTR7-EGFP were pre-treated with O4I3 (250 nM) for 2 days and then with O4I4 (20 μM) in combination with episomal expression of SKM (CSKM). B) GFP-positive colonies could be detected in HF1-LTR7-EGFP treated with O4I4 (20 μM) or in combination. C) The number of TRA-1-60 positive colonies in human fibroblasts (HF1, HF2 and HF3) treated with OSKM, SKM or CSKM. D) Comparison of relative Ct-values of OCT4 expression (DeltaCt=Ct_{gene}−Ct_{actin}) analyzed by qRT-PCR. Actin was used as control. E) Expression of OCT4 and SOX2 in CSKM-iPSCs detected by immunostaining.

FIGS. 22A-22H Reprogramming of human fibroblasts without exogenous overexpression of OCT4. A) GFP-positive colonies could be detected in HF1-LTR7-EGFP treated with O4I4 (20 μM) or in combination. B) Illustration of hiPSCs generation without OCT4. HF1-LTR7-EGFP were pre-treated with O4I3 (250 nM) for 2 days and then with O4I4 (5 μM) in combination with episomal expression of SKML (CSKML). The medium was refreshed every day. O4I4 was added in the first 7 days. E8 medium was used at day 10. C) The number of TRA-1-60 positive colonies in human fibroblasts (HF1, HF2 and HF3) treated with OSKML, SKML or CSKML. D) Comparison of relative Ct-vaules of OCT4 expression (DeltaCt=Ct_{gene}−Ct_{actin}) analyzed by qRT-PCR. Actin was used as control. E) Expression of OCT4 and SOX2 in CSKML-iPSCs detected by immunostaining. F) Expression of OCT4 and other pluripotency markers in CSKML. PSC1-3 and NCCIT were used as positive control, while HF1 as negative control. G) Hypomethylation of OCT4 promoter in CSKM-iPSCs detected by bisulfite sequencing. H) Comparison of global gene expression pattern of CSKML-iPSCs to PSC and HF1.

FIG. 23A-23C ATAC-seq analysis for the comparison of chromatin accessibility in OSKML-mediated and CSKML-mediated reprogramming. A) Principal component comparison of HF1, HF1 treated with OSKML, HF1 treated with CSKML and iPSCs for 7 days, 14 days and 21 days. At 21 days TRA-1-60+ cells were collected for analysis. B) Comparison of global chromatin accessibility. C) Comparison of chromatin accessibility at the promoter region of OCT4.

FIGS. 24A-24I An essential role of HMGA1 in CSKML-mediated reprogramming. A) Heat map of top upregulated genes in O4I4-treated fibroblasts in comparison to those expressions in PSC. B) Comparison of chromatin accessibility at the promoter of HMGA1 in HF1 treated either with OSKML or CSKML for 7 days. C) Chemical Induction of HMGA1 is prior to OCT4 expression. HF1 was treated with O4I3 (0.5 μM) and O4I4 (10 μM) over the indicated period. The medium was changed very day. D) HMAG1 and OCT4 are co-expressed in CSKML-treated fibroblasts. E) HMGA1 siRNA blocks CSKML-induced OCT4 expression. HF1 was treated as indicated. Cells were collected at day 7 for immunoblot analysis. F) HMGA1 knockdown inhibits OCT4 expression in both CSKML- and OSKML-mediated reprogramming. HMGA1 siRNA (siH) was co-transfected with SKML. Cells were collected at day 5 for immunoblot analysis and qPCR. G) Occupation of HMGA1 at the promoter of OCT4 detected by CHIP-qPCR. HF1 was transfected with SKML. HMGA1 was co-transfected at day 0 and day 3. H) Comparison of the number of TRA-1-60 positive colony according to the day when HMGA1 was overexpressed. HMGA1 was transfected at indicated days and every 2-3 days the transfection was re-performed. I) Reprogramming efficiency of HSKML-induced reprogramming in three human fibroblasts calculated by the number of TRA-1-60 positive colony. HMGA1 was co-trasfected with SKML for 7 days. **: p<0.01 and ***: p<0.001.

FIGS. 25A-25F CSKML-mediated HMGA1 activation is dependent on BMP6-ID. A) Comparison of genes expression which up-regulated or down-regulated in CSKML-treated fibroblasts to these in SKML-treated obtained from RNA-Seq, as well as in O4I4-treated to mock treatment. The heat map shows genes up-regulated or down-regulated in both case. HF1 was treated with O4I4 (10 μM) or 0.1% DMSO as mock, or transfected with SKML or in the combination (CSKML) for 48 h. The total RNA was collected for RNA-seq. B) CSKML activates BMP-SMAD-ID signalling pathway. BMP receptor inhibitor DM, BMP antagonist Noggin or SMAD4 siRNA blocks this effect. Dorsophormin (DM) or Noggin (200 ng/mL), or SMAD4 siRNA was added together with the treatment of CSKML for 48 h. C) Overexpression of ID1/2 enhances expression of HMGA1 and OCT4. HF1 was transfected with ID1/2 or in the combination with O4I3/4 or CSKML for 48 h. D) BMP6 enhances the expression of OCT4 induced by CSKML, which was blocked by ID1/2 siRNAs. BMP6 with/without ID1/2 siRNAs was added during the treatment with CSKML in the first 72 h. E) Adding BMP6 or overexpression of ID1/2 enhances the number of TRA-1-60 positive colony. F) Illustration of CSKML-mediated OCT4 expression.

### DETAILED DESCRIPTION

### Terms and Definitions

The term pluripotent stem cell in the context of the present specification relates to cells with pluripotent abilities. Pluripotency is a continuum ranging from completely pluripotent (=omnipotent) to partially pluripotent (=multipotent) cells. Omnipotent cells can differentiate into any cell type of the body. Examples for omnipotent stem cells (=completely pluripotent) are embryonic stem cells. In contrast, adult stem cells (=partially pluripotent stem cell) can usually only differentiate into the cell type of the organ they originate from. Multipotent cells have the gene activation potential to differentiate into discrete cell types. A non-limiting example of multipotent cells are neural stem cells. In addition to pluripotency, the pluripotent stem cell has the ability to go through several cycles of cell division while maintaining its undifferentiated state. The term pluripotent stem cell therefore encompasses such cells as embryonic stem cells, adult stem cells, neural stem cells and induced pluripotent stem cells.

The term C₁-C₃ alkyl in the context of the present specification relates to a saturated hydrocarbon having 1, 2 or 3 carbon atoms. Non-limiting examples for a C₁-C₃ alkyl are methyl, ethyl and n- or iso-propyl.

The term C₁-C₃ alkoxy moiety in the context of the present specification relates to a C₁-C₃ alkyl as specified above, connected to the core moiety by an oxygen. Examples of a C₁-C₃ alkoxy are methoxy (CH₃O—), ethoxy (CH₃CH₂O—) and n-propoxy (CH₃CH₂CH₂O—) or iso-propoxy ((CH₃)₂CHO—).

A C₁-C₃ carboxylic acid in the context of the present specification relates to a linear or branched hydrocarbon having 1, 2 or 3 carbon atoms and comprising a carboxyl group (—C(═O)OH). Non-limiting examples for a C₁-C₃ carboxylic acid are carbonic acid (OH—COOH), methanoic acid (H—COOH), acetic acid (CH₃—COOH) and propionic acid (CH₃CH₂COOH).

The term C₁-C₄ alkyl in the context of the present specification relates to a saturated linear or branched hydrocarbon having 1, 2, 3 or 4 carbon atoms, wherein in certain embodiments one carbon-carbon bond may be unsaturated and one CH₂ moiety may be exchanged for oxygen (ether bridge) or nitrogen (NH, or NR with R being methyl, ethyl, or propyl; amino bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl. In certain embodiments, a C₁-C₄ alkyl is a methyl, ethyl, propyl or butyl moiety.

A C₁-C₆ alkyl in the context of the present specification relates to a saturated linear or branched hydrocarbon having 1, 2, 3, 4, 5 or 6 carbon atoms, wherein one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples for a C₁-C₆ alkyl include the examples given for C₁-C₄ alkyl above, and additionally 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-inyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety.

The term C₄-C₇ cycloalkyl in the context of the present specification relates to a saturated hydrocarbon ring having 4, 5, 6 or 7 carbon atoms, wherein in certain embodiments, one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples of a C₄-C₇ cycloalkyl moiety include cyclobutyl (—C₄H₇), cyclopentenyl (C₅H₉), and cyclohexenyl (C₆H₁₁) moieties. In certain embodiments, a cycloalkyl is substituted by one C₁ to C₄ unsubstituted alkyl moiety. In certain embodiments, a cycloalkyl is substituted by more than one C₁ to C₄ unsubstituted alkyl moieties.

The term unsubstituted C, alkyl when used herein in the narrowest sense relates to the moiety —CₙH₂ₙ— if used as a bridge between moieties of the molecule, or —CₙH₂ₙ₊₁ if used in the context of a terminal moiety. It may still contain fewer H atoms if a cyclical structure or one or more (non-aromatic) double bonds are present.

The term Cₙ alkylene in the context of the present specification relates to a saturated linear or branched hydrocarbon comprising one or more double bonds. An unsubstituted alkylene consists of C and H only. A substituted alkylene may comprise substituents as defined herein for substituted alkyl.

The term Cₙ alkylyne in the context of the present specification relates to a saturated linear or branched hydrocarbon comprising one or more triple bonds and may also comprise one or more double bonds in addition to the triple bond(s). An unsubstituted alkylyne consists of C and H only. A substituted alkylyne may comprise substituents as defined herein for substituted alkyl.

The term “substituted” refers to the addition of a substituent group to a parent compound.

“Substituent groups” can be protected or unprotected and can be added to one available site or to many available sites in a parent compound. Substituent groups may also be further substituted with other substituent groups and may be attached directly or by a linking group such as an alkyl or hydrocarbyl group to a parent compound. “Substituent groups” useful in the context of alkyl or oxyalkyl substitution as disclosed herein include, without limitation, halogen, oxygen, nitrogen, sulphur, hydroxyl, alkyl, alkenyl, alkynyl, acyl (—C(O)R^{a}), carboxyl (—C(O)OR^{a}), aliphatic groups, alicyclic groups, alkoxy, substituted oxy (—OR^{a}), aryl, aralkyl, heterocyclic radical, heteroaryl, heteroarylalkyl, amino (—N(R^{b})(R^{c})), imino(=NR^{b}), amido (—C(O)N(R^{b})(R^{c}) or —N(R^{b})C(O)R^{a}), hydrazine derivates (—C(NH)NR^{a}R^{b}), tetrazole(CN₄H₂), azido (—N₃), nitro (—NO₂), cyano (—CN), isocyano (—NC), cyanato (—OCN), isocyanato (—NCO), thiocyanato (—SCN); isothiocyanato (—NCS); carbamido (—OC(O)N(R^{b})(R^{c}) or —N(R^{b})C(O)OR^{a}), thiol (—SR^{b}), sulfinyl (—S(O)R^{b}), sulfonyl (—S(O)₂R^{b}), sulfonamidyl (—S(O)₂N(R^{b})(R^{c}) or —N(R^{b})S(O)₂R^{b}) and fluorinated compounds —CF₃, —OCF₃, —SCF₃, —SOCF₃ or —SO₂CF₃, wherein each R^{a}, R^{b} and R^{c} is, independently from any other R^{a}, R^{b} and R^{c}, H or a further substituent group with a preferred list including without limitation, C₁-C₃ alkyl alkyl, C₂-C₃ alkenyl, C₂-C₃ alkynyl, alkoxy, C₁-C₃ acyl, aryl, heteroaryl, alicyclic, heterocyclic and heteroarylalkyl.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable-linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, choride Cl, bromide Br, iodide I, phosphonate PO₃H₂, PO₃R₂, phosphate OPO₃H₂ and OPO₃R₂, sulfhydryl SH, suflalkyl SR, sulf oxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term amino substituted alkyl or hydroxyl substituted alkyl relates to an alkyl according to the above definition that is modified by one or several amine or hydroxyl groups NH₂, NHR, NR₂ or OH, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified. An alkyl having more than one carbon may comprise more than one amine or hydroxyl. Unless otherwise specified, the term “substituted alkyl” refers to alkyl in which each C is only substituted by at most one amine or hydroxyl group, in addition to bonds to the alkyl chain, terminal methyl, or hydrogen.

The term carboxyl substituted alkyl refers to an alkyl according to the above definition that is modified by one or several carboxyl groups COOH, or derivatives thereof, particularly carboxylamides CONH₂, CONHR and CONH₂, or carboxylic esters COOR, with R having the meaning as laid out in the preceding paragraph and different from other meanings assigned to R in the body of this specification.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —(CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂ and —CH₂CH(CH₂NHEt)₂ for terminal moieties and —CH₂CHNH₂—, —CH₂CHNHMe-, —CH₂CHNHEt- for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

The term halogen-substituted alkyl refers to an alkyl according to the above definition that is modified by one or several halogen atoms selected (independently) from F, Cl, Br, I.

The term fluoro substituted alkyl refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

Non-limiting examples of hydroxyl- and fluoro-substituted alkyl include —CHFCH₂OH, —CF₂CH₂OH, —(CHF)₂CH₂OH, —(CF₂)₂CH₂OH, —(CHF)₃CH₂OH, —(CF₂)₃CH₂OH, —(CH₂)₃OH, —CF₂CH(OH)CH₃, —CF₂CH(OH)CF₃, —CF(CH₂OH)CHFCH₃, and —CF(CH₂OH)CHFCF₃.

The term aryl in the context of the present specification relates to a cyclic aromatic C₅-C₁₀ hydrocarbon that may comprise a heteroatom (e.g. N, O, S). Examples of aryl include, without being restricted to, phenyl and naphthyl, and any heteroaryl. A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. An aryl or a heteroaryl in the context of the specification additionally may be substituted by one or more alkyl groups.

An aryl methylene in the context of the present specification relates to a CH₂ (-methylene) group substituted by an aryl moiety. One non-limiting example of aryl methylene is a benzyl (Bn) group. If used in particular, a heteroaryl methylene in the context of the present specification signifies a CH₂ (-methylene) group substituted by a heteroaryl moiety. Examples for heteroaryl include, without being restricted to, pyrrole, thiophene, furan, imidazole, pyrazole, thiazole, oxazole, pyridine, pyrimidine, thiazin, quinoline, benzofuran and indole. An aryl or a heteroaryl in the context of this specification additionally may be substituted by one or more alkyl groups.

A first aspect of the invention relates to a method of producing a pluripotent stem cell, in particular an induced pluripotent stem cell. This method comprises contacting a non-pluripotent donor cell obtained from a mammalian donor with a compound characterized by general formula (1).

Certain embodiments of the present invention do not encompass the manipulation of cells in-vivo, but is practiced in-vitro, and does not relate to the manipulation of primates, particularly humans, or to the manipulation of primate, particularly human, embryos.

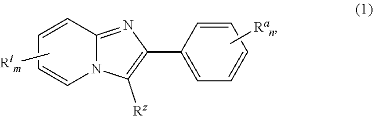

The substituent R^{I} is attached to the six-membered ring of the pyrimidazole (imidazo[1,2-A]pyridine) moiety. The point of attachment might be any of the four carbon atoms available for substitution. The index m of R^{I}ₘ indicates the number of substituents that are attached to the pyrimidazole moiety. The value of m is 1 or 2.

The substituent R^{I} is selected from —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃.

In certain embodiments, the substituents are selected from halogen, oxygen, nitrogen, sulphur and hydroxyl.

The substituent R^{z} is selected from general formula (2) or hydrogen.

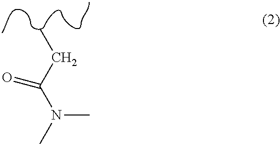

In certain embodiments, the compound according to aspect 1 is zolpidem (CAS No. 82626-48-0), with m of R^{I}ₘ being 1, R^{I} being CH₃, R^{z} being general formula (2), n of R^{a}ₙ being 1 and R^{a} being CH₃.

The substituent R^{a} is attached to the aryl moiety. The point of attachment might be any of the six carbon atoms of the aryl moiety. The index n of R^{a}ₙ indicates the number of substituents that are attached to the aryl moiety. The value of n is 1 or 2.

The substituent R^{a}, independently from each other R^{a}, is selected from —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃. R^{a} may also take any of the values given for R′, as well.

In certain embodiments, two R^{a} bonded to two adjacent carbon atoms in the aryl moiety form a circular structure via the hydrocarbon C₄H₄ (the moiety attached to the imidazole ring is a naphtyl)

The carbon atom of the aryl moiety that is linked to the imidazole moiety is not substituted by R^{a}.

In certain embodiments, the value of m is 1.

In certain embodiments, the value of m is 2.

In certain embodiments, the value of n is 1.

In certain embodiments, the value of n is 2.

In certain embodiments, R′ is methyl.

In certain embodiments, R^{a} is selected from —H, —CH₃, —F, —Cl, —Br, —I, —OH, —NO₂, —CN or —OMe.

In certain embodiments, R^{a} is methyl.

In certain embodiments, the compound used in the method according to the first aspect of the invention is characterized by general formula (3).

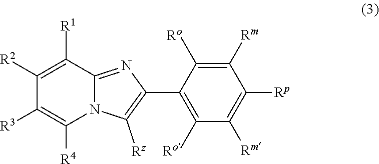

One of R¹, R², R³ and R⁴ is selected from: —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃. The other ones are —H. In other words, only one of the substituents R¹, R², R³ and R⁴ is bonded to the pyridine moiety. The meaning of the substituents is according to the definition provided above.

The substituent R^{z} is selected from general formula (2) or hydrogen.

One of R^{o}, R^{m}, R^{p}, R^{m′} and R^{o′} is selected from: —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃. The other ones are —H. In other words, only one of the substituents R^{o}, R^{m}, R^{p}, R^{m′} and R^{o′} is bonded to the aryl moiety. The meaning of the substituents is according to the definition provided above.

In certain embodiments, any two neighbouring positions of R^{o}, R^{m}, R^{p}, R^{m′} or R^{o′} together form a circular structure via the hydrocarbon C₄H₄.

In certain embodiments, R^{m} and R^{p} together form a circular structure via the hydrocarbon C₄H₄.

In certain embodiments, each R^{I}, R^{a}, R¹, R², R³, R⁴, R^{o}, R^{m}, R^{p}, R^{m′} and R^{o′} is selected independently from each other from —H, —CH₃, —F, —Cl, —Br, —I, —OH, —NO₂, —CN or —OMe.

In certain embodiments, R¹ is selected from —CH₃, —H.

In certain embodiments, R² is selected from —CH₃, —H.

In certain embodiments, R³ is selected from —CH₃, —H, —F, —Cl, —Br.

In certain embodiments, R⁴ is selected from —CH₃, —H.

In certain embodiments, R^{o′} is selected from —NO₂, —F, —H.

In certain embodiments, R^{o} is selected from —NO₂, —F, —H.

In certain embodiments, R^{p} is selected from —CH₃, —F, —Cl, —Br, —NO₂, —OMe, —OH, —CN, —H.

In certain embodiments, R^{m} is selected from —C₁, —H, —F, —OH, —NO₂, —OMe.

In certain embodiments, R^{m′} is selected from —C₁, —H, —F, —OH, —NO₂, —OMe.

In certain embodiments, R^{z} is selected from —H or formula (2).

In certain embodiments, the compound is N,N-dimethyl-2-(6-methyl-2-p-tolylimidazo[1,2-a]pyridin-3-yl)acetamid (100).

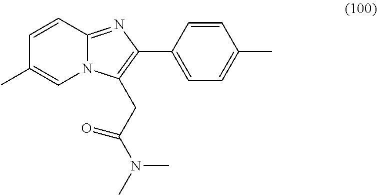

### [Zolpidem; CAS No. 82626-48-0].

In certain embodiments, the compound is 6-methyl-2-(p-tolyl)imidazo[1,2-a]pyridine (101).

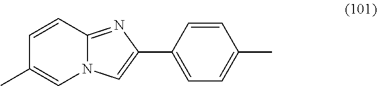

In certain embodiments, the compound is 2-(4-fluorophenyl)-6-methyl-imidazo[1,2-a]pyridine (102).

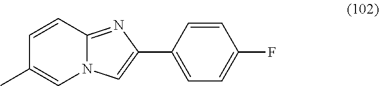 [SMILES: Cc1ccc2nc(-c3ccc(F)cc3)cn2c1]

In certain embodiments, the compound is 2-(4-chlorophenyl)-6-methyl-imidazo[1,2-a]pyridine (103).

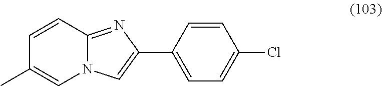 [SMILES: Cc1ccc2nc(-c3ccc(Cl)cc3)cn2c1]

In certain embodiments, the compound is 2-(4-bromophenyl)-6-methyl-imidazo[1,2-a]pyridine (104).

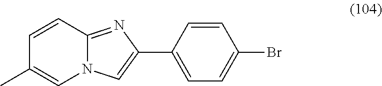 [SMILES: Cc1ccc2nc(-c3ccc(Br)cc3)cn2c1]

In certain embodiments, the compound is 6-methyl-2-(4-nitrophenyl)imidazo[1,2-a]pyridine (105).

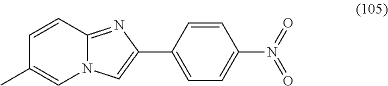 [SMILES: Cc1ccc2nc(-c3ccc([N+](=O)[O-])cc3)cn2c1]

In certain embodiments, the compound is 2-(4-methoxyphenyl)-6-methyl-imidazo[1,2-a]pyridine (106).

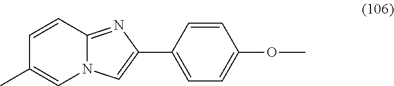

In certain embodiments, the compound is 4-(6-methylimidazo[1,2-a]pyridin-2-yl)phenol (107).

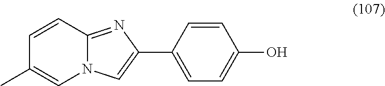 [SMILES: Cc1ccc2nc(-c3ccc(O)cc3)cn2c1]

In certain embodiments, the compound is 4-(6-methylimidazo[1,2-a]pyridin-2-yl)benzonitrile (108).

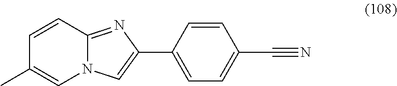 [SMILES: Cc1ccc2nc(-c3ccc(C#N)cc3)cn2c1]

In certain embodiments, the compound is 6-methyl-2-phenyl-imidazo[1,2-a]pyridine (109).

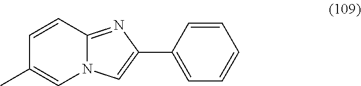 [SMILES: Cc1ccc2nc(-c3ccccc3)cn2c1]

In certain embodiments, the compound is 6-methyl-2-(3-nitrophenyl)imidazo[1,2-a]pyridine (110).

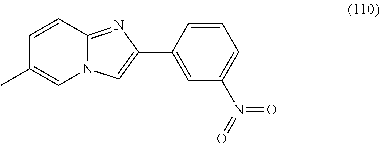 [SMILES: Cc1ccc2nc(-c3cccc([N+](=O)[O-])c3)cn2c1]

In certain embodiments, the compound is 6-methyl-2-(2-nitrophenyl)imidazo[1,2-a]pyridine (111).

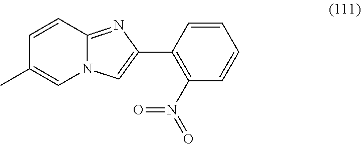 [SMILES: Cc1ccc2nc(-c3ccccc3[N+](=O)[O-])cn2c1]

In certain embodiments, the compound is 2-(3-fluorophenyl)-6-methyl-imidazo[1,2-a]pyridine (112).

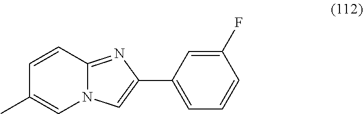 [SMILES: Cc1ccc2nc(-c3cccc(F)c3)cn2c1]

In certain embodiments, the compound is 2-(2-fluorophenyl)-6-methyl-imidazo[1,2-a]pyridine (113).

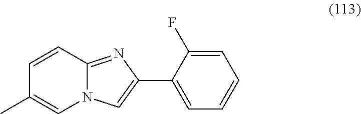 [SMILES: Cc1ccc2nc(-c3ccccc3F)cn2c1]

In certain embodiments, the compound is 2-(3-chlorophenyl)-6-methyl-imidazo[1,2-a]pyridine (114).

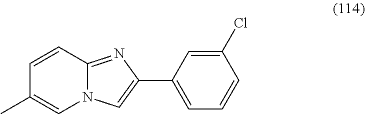 [SMILES: Cc1ccc2nc(-c3cccc(Cl)c3)cn2c1]

In certain embodiments, the compound is 3-(6-methylimidazo[1,2-a]pyridin-2-yl)phenol (115).

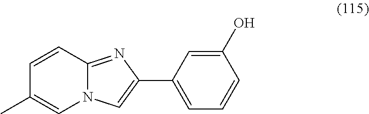 [SMILES: Cc1ccc2nc(-c3cccc(O)c3)cn2c1]

In certain embodiments, the compound is 2-(3-methoxyphenyl)-6-methyl-imidazo[1,2-a]pyridine (116).

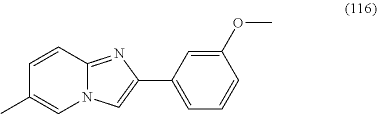

In certain embodiments, the compound is 7-methyl-2-(p-tolyl)imidazo[1,2-a]pyridine (117).

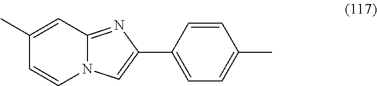

In certain embodiments, the compound is 8-methyl-2-(p-tolyl)imidazo[1,2-a]pyridine (118).

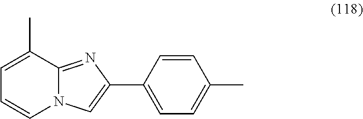 [SMILES: Cc1ccc(-c2cn3cccc(C)c3n2)cc1]

In certain embodiments, the compound is 5-methyl-2-(p-tolyl)imidazo[1,2-a]pyridine (119).

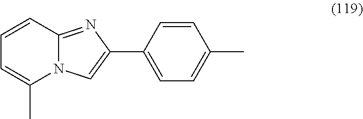 [SMILES: Cc1ccc(-c2cn3c(C)cccc3n2)cc1]

In certain embodiments, the compound is 6-chloro-2-(p-tolyl)imidazo[1,2-a]pyridine (120).

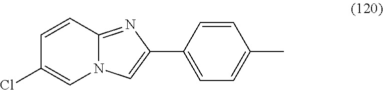 [SMILES: Cc1ccc(-c2cn3cc(Cl)ccc3n2)cc1]

In certain embodiments, the compound is 6-fluoro-2-(p-tolyl)imidazo[1,2-a]pyridine (121)

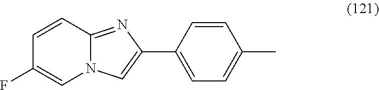 [SMILES: Cc1ccc(-c2cn3cc(F)ccc3n2)cc1]

In certain embodiments, the compound is 6-bromo-2-(p-tolyl)imidazo[1,2-a]pyridine (122).

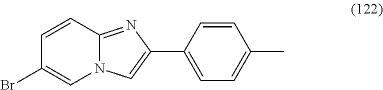 [SMILES: Cc1ccc(-c2cn3cc(Br)ccc3n2)cc1]

In certain embodiments, the compound is 6-methyl-2-(2-naphthyl)imidazo[1,2-a]pyridine (123).

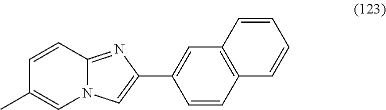 [SMILES: Cc1ccc2nc(-c3ccc4ccccc4c3)cn2c1]

An alternative of this first aspect of the invention relates to a method of producing a pluripotent stem cell, in particular a method of producing an induced pluripotent stem cell, wherein the method comprises contacting a non-pluripotent donor cell obtained from a mammalian (particularly a human) donor with a compound characterized by general formula (4):

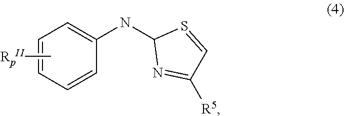

Therein, each R^{II}, independently from any other R^{II}, is selected from
—H,
substituted or unsubstituted C₁-C₃ alkyl, particularly methyl,
substituted or unsubstituted C₁-C₃ alkoxy, particularly O—CH₃,
—F, —Cl, —Br, —I, —NO₂, —OH, —NH₂, —SO₃H, —CN, —NCO, —CNO, —NCS, —SCN, particularly —F, —Cl, —Br, —NO₂, —OH, and —CN,
substituted or unsubstituted C₁-C₃ carboxylic acid, —CH₂F, —CHF₂, or —CF₃, particularly COOH.

p is 1, 2 or 3, particularly p is 1 or 2, and R⁵ is selected from
—H,
substituted or unsubstituted C₁-C₄ alkyl, particularly t-butyl,
substituted or unsubstituted aryl, particularly unsubstituted phenyl or naphtyl;
COOR, wherein R is an unsubstituted or substituted C₁-C₄ alkyl, particularly R is an unsubstituted C₁-C₄ alkyl, more particularly R is ethyl;

The following combinations are not encompassed in the definition of compound (4):
R⁵ is COOR with R being an unsubstituted or substituted C₁-C₄ alkyl, particularly R is an unsubstituted C₁-C₄ alkyl, more particularly R is ethyl, and
R^{II} is halogen or hydrogen and p is selected from 1 or 2.

In certain embodiments, the compound according to formula (4) is the compound according to formula (5), (6) or (7):

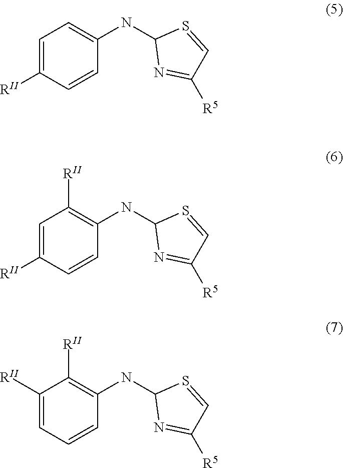

wherein R^{II} and R⁵ have the same meanings as indicated above.

In certain embodiments, each R^{II}, independently from any other R^{II}, is selected from methyl and ethyl. In certain particular embodiments, all R^{II} are methyl.

In certain embodiments, R⁵ is selected from substituted or unsubstituted C₃-C₄ alkyl. In certain particular embodiments, R⁵ is unsubstituted t-butyl.

In certain embodiments, R⁵ is selected from substituted or unsubstituted aryl. In certain particular embodiments, R⁵ is unsubstituted phenyl or naphtyl.

In certain embodiments, the compound according to formula (4) is selected from any one of the formulae (419), (420), (421), (422) and (423). A particular embodiment of the method of the invention uses the compound according to formula (423)

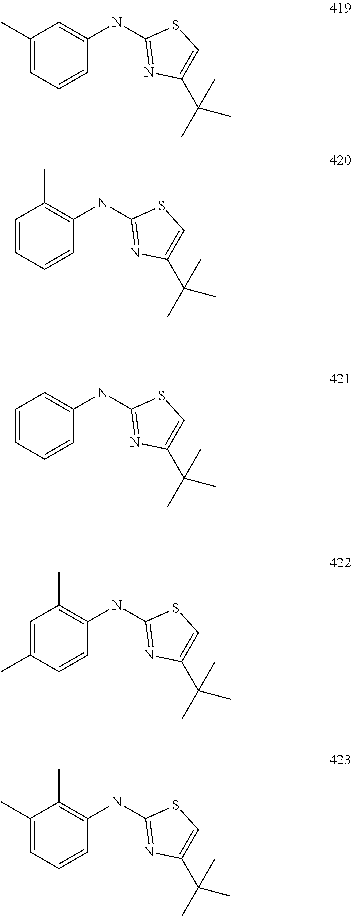

In certain embodiments, the mammalian donor is a human, in other words, a cell of human origin is modified.

In certain embodiments, the method according to the first aspect of the invention further comprises introducing into the mammalian non-pluripotent donor cell at least one element selected from:
a nucleic acid sequence encoding a polypeptide,
a polypeptide, and
a non-coding nucleic acid sequence.

The polypeptide is selected from:
the family of OCT polypeptides,
the family of KLF polypeptides,
the family of SOX polypeptides,
the family of MYC polypeptides, or
the family of LIN polypeptides.

In certain embodiments, the polypeptide is OCT4.

In certain embodiments, the polypeptide is KLF4.

In certain embodiments, the polypeptide is SOX2.

In certain embodiments, the polypeptide is L-MYC.

In certain embodiments, the polypeptide is Lin28.

The non-coding nucleic acid sequence is selected from: miR-302b, miR-372, miR-302, miR-367, miR-200c and miR-369.

In certain embodiments, a multitude of nucleic acid sequences encoding a polypeptide, polypeptides or non-coding nucleic acid sequences are introduced into the mammalian non-pluripotent donor cell. In other words, one “element” can comprise several nucleic acid sequences or polypeptides within the same category of the “element”.

In certain embodiments, the compound is used in a final concentration between 0.0005 μmol/l and 100 μmol/l.

In certain embodiments, the compound is used in a final concentration between 0.001 μmol/l and 15 μmol/l.

In certain embodiments, the compound is used in a final concentration between 0.001 μmol/l and 10 μmol/l.

According to a second aspect of the invention, a method of inducing OCT4 and NANOG in a mammalian cell is provided. The method is comprising contacting a mammalian donor cell with a compound according to the first aspect of the invention.

According to a third aspect of the invention a method of increasing methylation of histone 3 lysine 4 (H3K4) in a mammalian non-pluripotent donor cell to augment a method of producing an induced pluripotent stem cell is provided. The method is comprising contacting the donor cell with a compound according to the first aspect of the invention.

It is known in the art that fibroblasts may become resistant to reprogramming approaches to induce pluripotency. One condition known to increase the capacity of a donor cell to be reprogrammed is the maintenance of an open chromatin state that allows for transcription factor binding. In this context trimethylation of histone H3 lysine 4 (H3K4Me3) is reported to provide such an open chromatin state (A. Soufi, G. Donahue and K. S. Zaret, Cell, 2012, 151, 994-1004; Koche, et al., Cell Stem Cell, 2011, 8, 96-105; Polo and Hochedlinger, Cell Stem Cell, 2010, 7, 5-6).

According to an alternative aspect of the invention, a method for augmenting a method of producing an induced pluripotent stem cell is provided. The method is comprising contacting the donor cell with a compound according to the first aspect of the invention.

In certain embodiments according to the first, second and third aspect of the invention, a compound is used in its final concentration for a duration of 12 hours to 10 days.

In certain embodiments according to the first, second and third aspect of the invention, a compound is used in its final concentration for a duration of 1 day to 8 days.

In certain embodiments according to the first, second and third aspect of the invention, a compound is used in its final concentration for a duration of 2 days to 7 days.

In certain embodiments according to the first, second and third aspect of the invention, a compound is used in its final concentration for a duration of 2 to 8 months.

According to a fourth aspect of the invention, a method of maintaining pluripotency in mammalian pluripotent stem cells is provided. The method is comprising contacting the mammalian pluripotent stem cell with a compound according to the first aspect of the invention.

In certain embodiments, the mammalian pluripotent stem cell is a multipotent neural stem cell.

In certain embodiments, according to all aspects of the invention, the method is practiced in vitro or ex vivo.

In certain embodiments, according to all aspects of the invention the compound is used in a final concentration between 0.0005 μmol/l and 100 μmol/l.

In certain embodiments, according to all aspects of the invention the compound is used in a final concentration between 0.001 μmol/l and 15 μmol/l.

In certain embodiments, according to all aspects of the invention the compound is used in a final concentration between 0.001 μmol/l and 10 μmol/l.

According to a fifth aspect of the invention, an induced pluripotent mammalian stem cell obtained or obtainable by a method according to any one of the other aspects of the invention is provided.

According to a sixth aspect of the invention, a cell culture medium comprising a compound according to the first aspect of the invention is provided.

In certain embodiments, the compound is used in a final concentration between 0.0005 μmol/l and 100 μmol/l.

In certain embodiments, the compound is used in a final concentration between 0.001 μmol/l and 15 μmol/l.

In certain embodiments, the compound is used in a final concentration between 0.001 μmol/l and 10 μmol/l.

According to a seventh aspect of the invention, a composition for promoting formation of induced pluripotent mammalian stem cells is provided. The composition is comprising a compound according to the first aspect of the invention.

The invention is further illustrated by the following examples and figures, from which further embodiments and advantages can be drawn. These examples are meant to illustrate the invention but not to limit its scope.

### Examples

Drug repurposing provides a successful way to develop new therapies with known, medically approved drugs within remarkably short periods. The inventors intentionally sought for chemicals sharing backbones with approved drugs with the expectation of having advantages in pharmacodynamics and kinetics.

In an effort to find such chemical structures able to induce pluripotency, a high-throughput screening (HTS) was performed. The screen was based on cellular luciferase reporter assays under the control of the promoter activity of a pluripotency-associated transcription factor (Oct4) in HEK293 cells. About 4000 imidazopyridine derivatives with significantly differential luciferase activity were identified in this screen.

All methods and reagents used in these examples are disclosed below.

### Synthesis of Lead Compound:

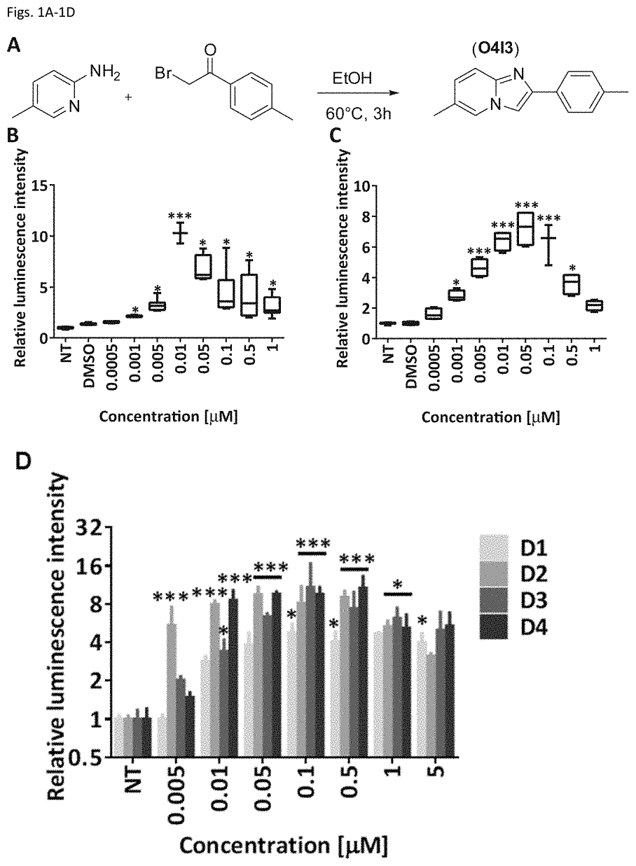

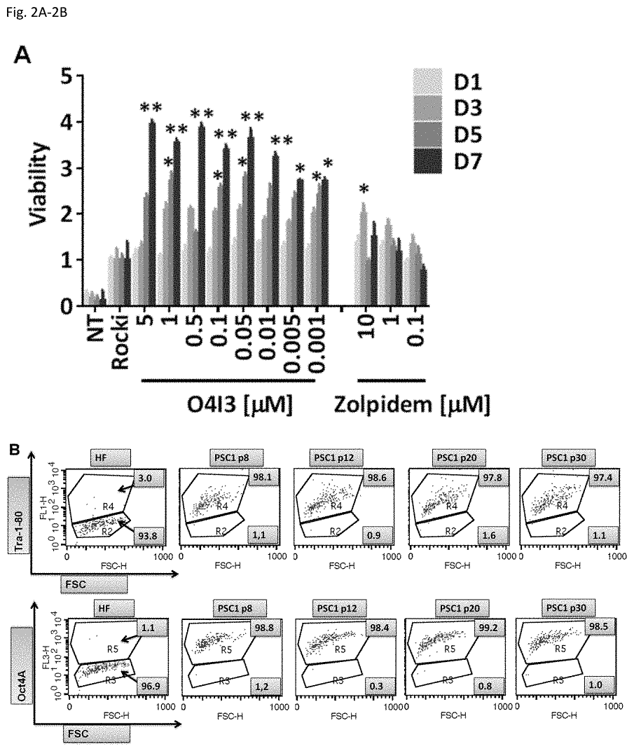

To study the activating and inhibitory effects of the identified compounds in detail, one of the lead structures, 6-methyl-2-(p-tolyl)imidazo[1,2-a]pyridine (O4I3; (101)), was selected and synthesized. This was achieved by heating ω-bromoacetophenone with an excess of 2-aminopyridine for 3 h (FIG. 1A), achieving an excellent yield (>95%), because the product could be readily isolated from the reaction mixture according to distinct solubility under acidic conditions.

### Effect of Lead Compound (O4I3) and Zolpidem on Oct4

The ability of the lead compound to induce Oct4 at translational level was tested in a luciferase reporter cell line in human embryonic carcinoma NCCIT cells driven by Oct4 response elements (NCCIT-Oct4). The compound lead to a nearly 10-fold induction of Oct4 at 10 nM compared to untreated cells (FIG. 1B). Consequently, the lead compound was named “Oct4 inducing compound 3” (O4I3).

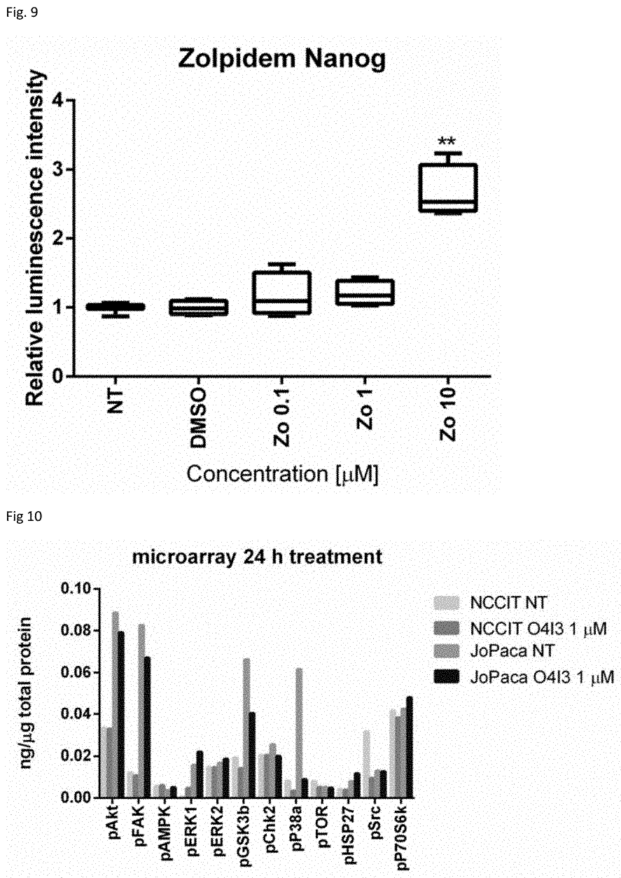

It is known in the art that Nanog is a direct target of Oct4 and can be activated by other Oct4 inducing compounds. Accordingly, a similar pattern of activation was observed in O4I3-treated NCCIT-Nanog reporter cells, carrying a Nanog response element (FIG. 1C). At higher concentrations of O4I3 (FIGS. 1B and 1C) less activity was observed. Moreover, the activity of zolpidem (N,N-Dimethyl-2-(6-methyl-2-p-tolylimidazo[1,2-a]pyridin-3-yl)acetamid) (100) containing a similar backbone to O4I3 was tested in both reporter cell lines. An approximate 2-fold induction at 10 μM was detected (FIG. 8 and FIG. 9). Notably, O4I3 (1 nM) could achieve a comparable induction at a 10,000-fold lower concentration. Collectively, these results documented that O4I3 and zolpidem activated Oct4 at both transcriptional and translational level.

### Time Course of Oct4 Activation

To address the question whether short or longer-term activation is achieved, the induction of Oct4 was monitored over several days in these experiments. A continuous and constant activation over 4 days was observed in NCCIT-Oct4 cells treated with O4I3 (FIG. 1C). Without wishing to be bound by theory, the inventors believe that the lower activity at higher concentrations might be caused by activation of certain signaling pathways antagonizing pluripotency. Indeed, excessive Oct4 expression can lead to differentiation of embryonic stem cells (ESCs) to extra-embryonic endoderm and mesoderm.

In good agreement, the inventors found the induction of those markers, T and Gata4, in hiPSCs treated with 10 μM O4I3 (FIG. 11), suggesting that the differentiation was induced by O4I3 at high concentrations.

Effect on iPSC Self Renewal

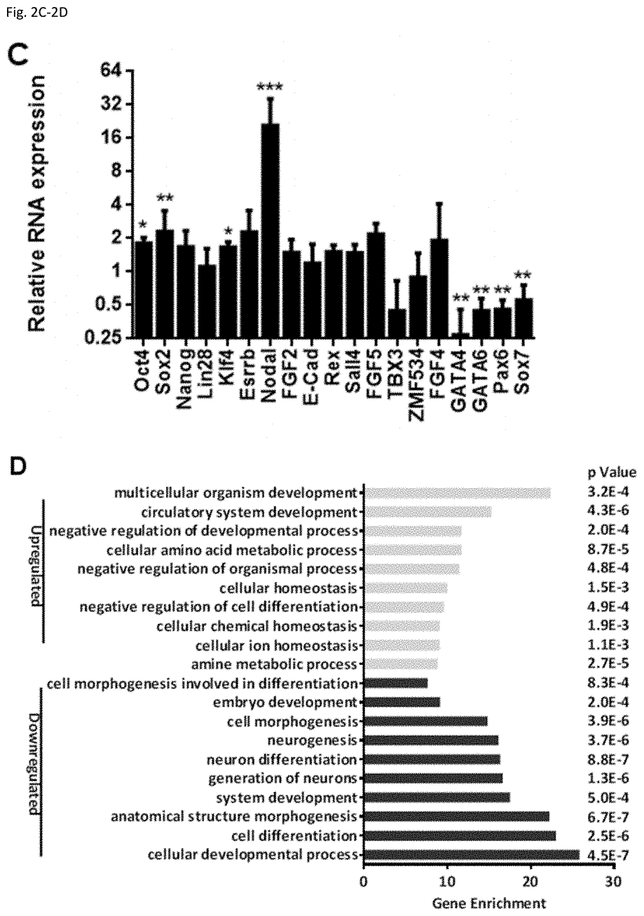

Since O4I3 induced sustainable expression of Oct4 and Nanog, the inventors questioned if it supports iPSC self-renewal in single cell expansion. Therefore, the viability of iPSCs, after replating Accutase®-dissociated single iPSCs, was tested in the presence or absence of O4I3. Rocki (Y-27632) was used for the first 24 h to increase iPSCs survival. A nearly 300% higher viability in cells treated with O4I3 (1 nM) was observed. Using 50 nM of O4I3 even 400% increase was observed (FIG. 2A). FACS analysis confirmed homologous expression of iPSCs markers, Tra-1-81 and Oct4, for at least 30 passages (FIG. 2B).

O4I3 Maintains Pluripotency of Human iPSCs

The cellular responses to O4I3 was analyzed in two stem cell lines, NCCIT and JoPaca-1 (FIG. 10). Using a phosphorylation microarray (Holenya et al, Proteomics. 2011 May; 11(10):2129-33) revealed that O4I3 selectively activated Erk1, an essential element for supporting mouse ESC self-renewal, but not Erk2, whose knockdown protects mouse ESC differentiation.

Moreover, inactive phosphorylation of GSK3β was observed. Without wishing to be bound by theory, the inventors believe that this is implicating activation of PI3K/Akt and Wnt, two signaling pathways that are fundamental for maintenance of human PSCs.

Inhibition of p38 and Src was also observed. Both are known in the art as constituents of chemical cocktails for conversion of human primed iPSCs to a naïve state.

In addition, zolpidem also sustained the maintenance of iPSCs, although at a much higher concentration and with lower efficacy than O4I3 (FIG. 2A).

Therefore, the inventors investigated if O4I3 can support human PSC self-renewal. The viability of previously well-characterized hiPSCs (thereafter called hPSCs) was investigated after replating Accutase®-dissociated single hPSCs in the presence/absence of O4I3, where Rocki (Y-27632) was used for the first 24 h to increase hPSCs survival. A nearly 300% higher viability was found in cells treated with O4I3 even at 1 nM, and 400% at 50 nM (FIG. 2A). FACS analysis confirmed homologous expression of PSCs markers, Tra-1-81 and Oct4, for at least 30 passages under feeder free condition (FIG. 2B). In addition, zolpidem also sustained the maintenance of iPSCs, although at a much higher concentration and with lower efficacy (FIG. 2A).

Gene expression analysis by qRT-PCR showed that O4I3 stabilized or slightly increased the expression of pluripotency-associated genes in iPSCs, including Oct4, Sox2, Nanog and Nodal, while expression of genes regulating differentiation was reduced, like Gata4, Gata6, Pax6 and Sox7 (FIG. 2C).

Functional annotation analysis of DNA microarray profiles of gene expression in O4I3-treated iPSCs showed that O4I3 inhibited cell differentiation, while maintained cellular homeostasis (FIG. 2D). Thus, the synergism of promoting iPSC proliferation and self-renewal, and in parallel inhibiting differentiation could explain the observed positive effect of O4I3 on iPSCs maintenances.

### Effects of O4I3 on Self-Renewal of Neural Stem Cells

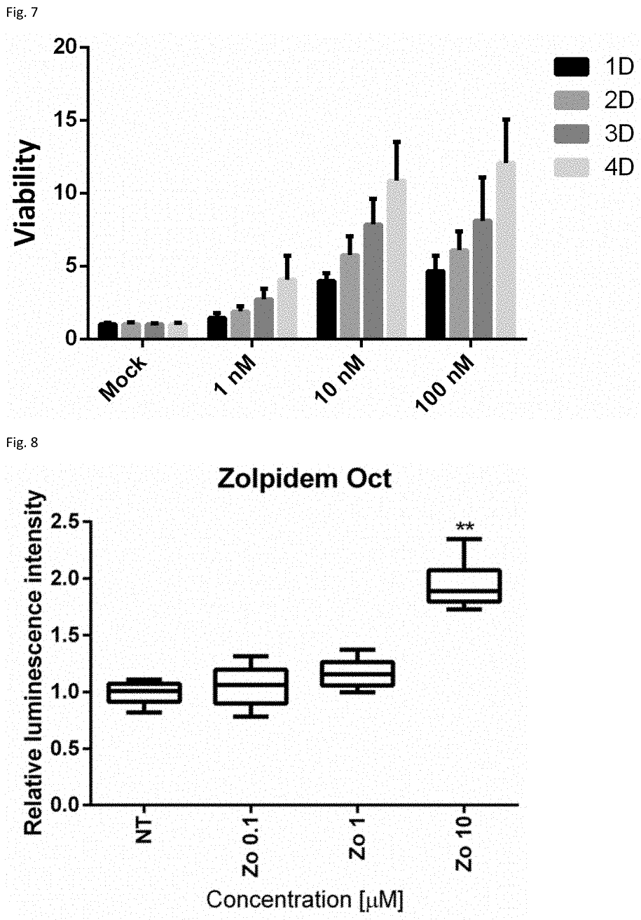

The self-renewal of multipotent neural stem cells was also tested with the compound of the current invention. Neural stem cells were cultivated in SPN medium with or without O4I3 at different final concentrations (FIG. 7). The MTT assay was applied to determine cell viability. Starting from a concentration of 1 nM O4I3 the viability of neural stem cells was increased in comparison to mock treated neural stem cells.

### Effects of O4I3 on Human Fibroblasts

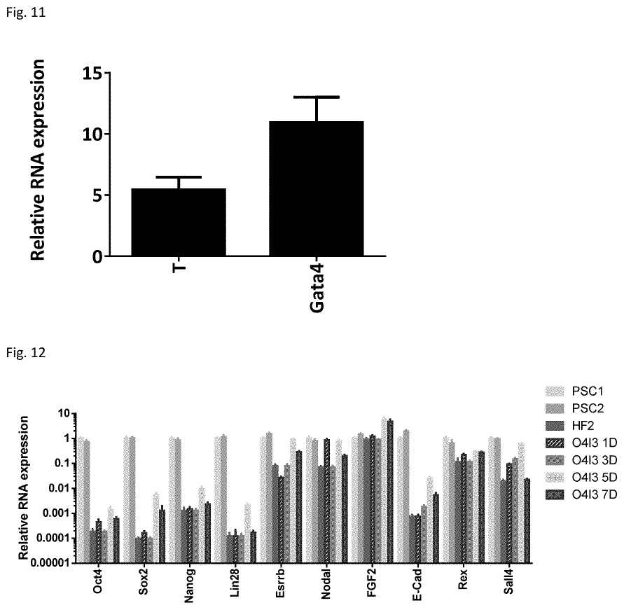

Analyzing the effects of O4I3 on human fibroblasts, the activation of reprogramming associated genes, including an about 10-fold induction of Oct4, Nanog and Lin28, and an about 100-fold increase of Sox2 and E-Cadherin mRNA levels was detected. In particular, expression of several essential genes for reprogramming is equal to those in pluripotent stem cells, such as Esrrb, FGF2 and Sall4 (FIG. 12).

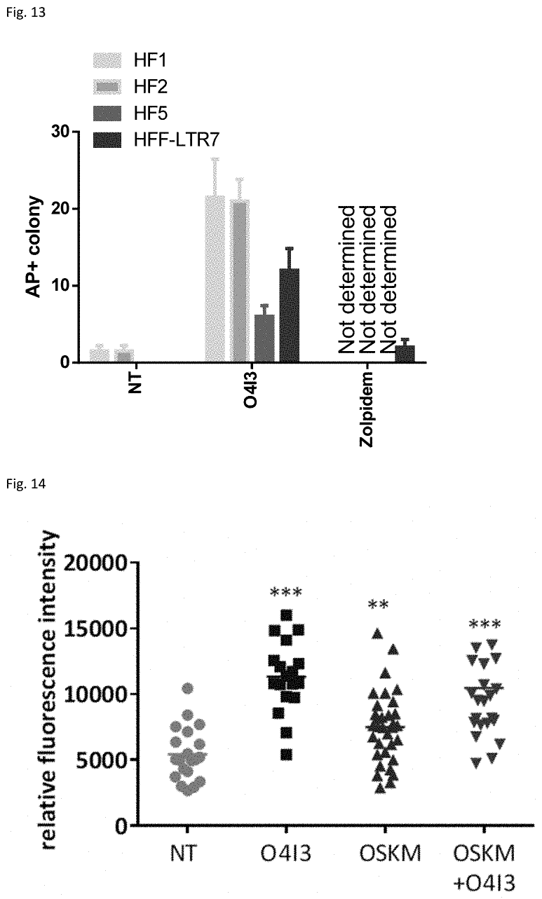

O4I3 was applied in commercially available episome-based reprogramming cocktail containing episomal vectors for overexpression of Oct4, Sox2, Klf4, Lin28 and L-Myc, as well as suppression of p53. In comparison to non-treated fibroblast cultures, 20 times more alkaline phosphatase (AP) positive colonies were detected in the presence of O4I3 at a concentration of 50 nM in two tested human primary fibroblasts (HF1 and HF2) (FIG. 13).

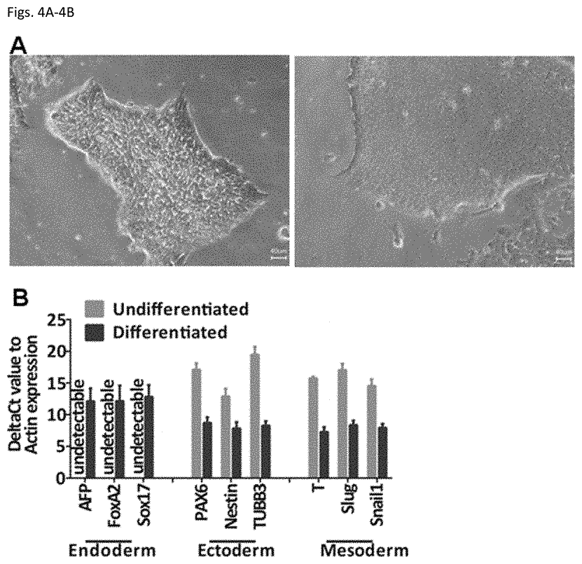

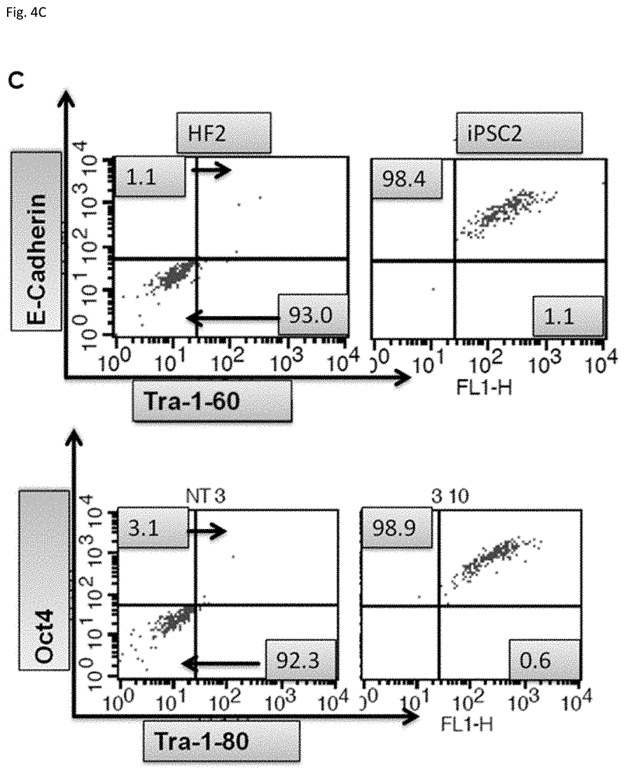

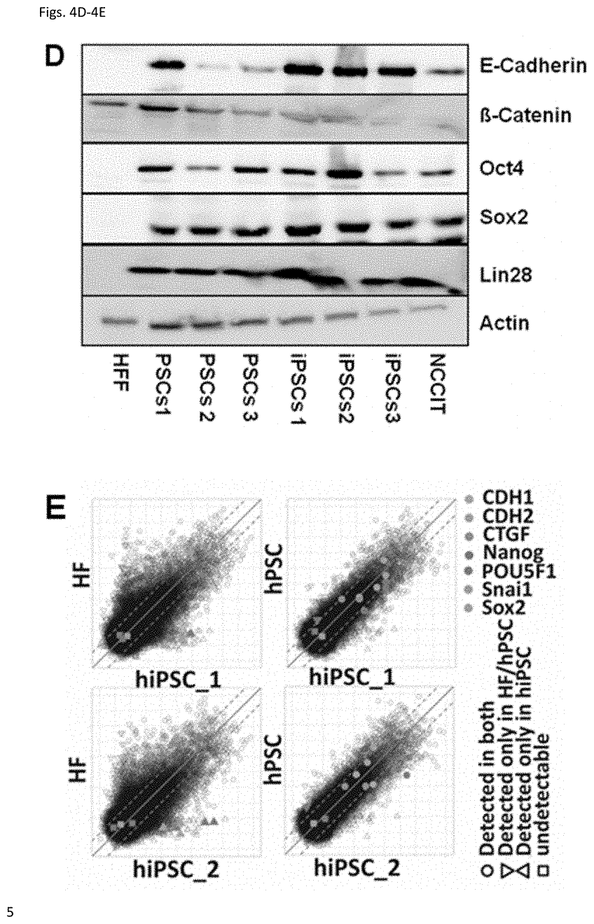

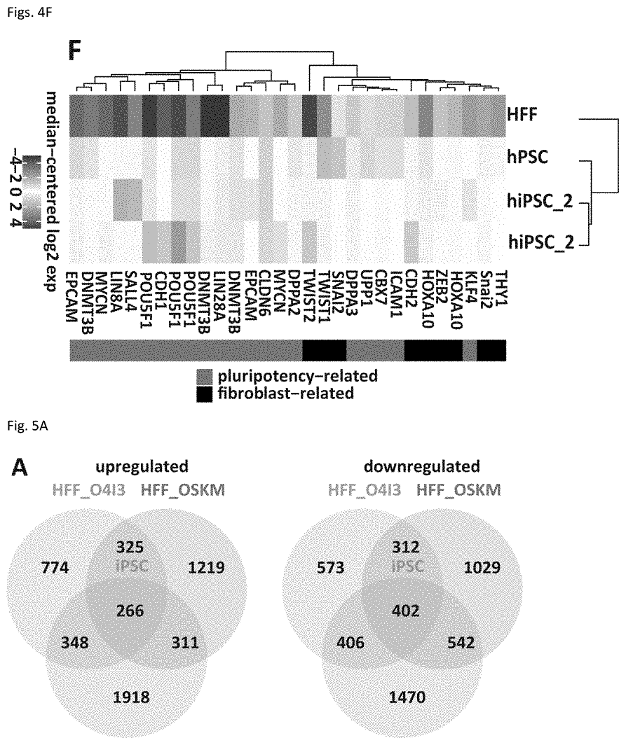

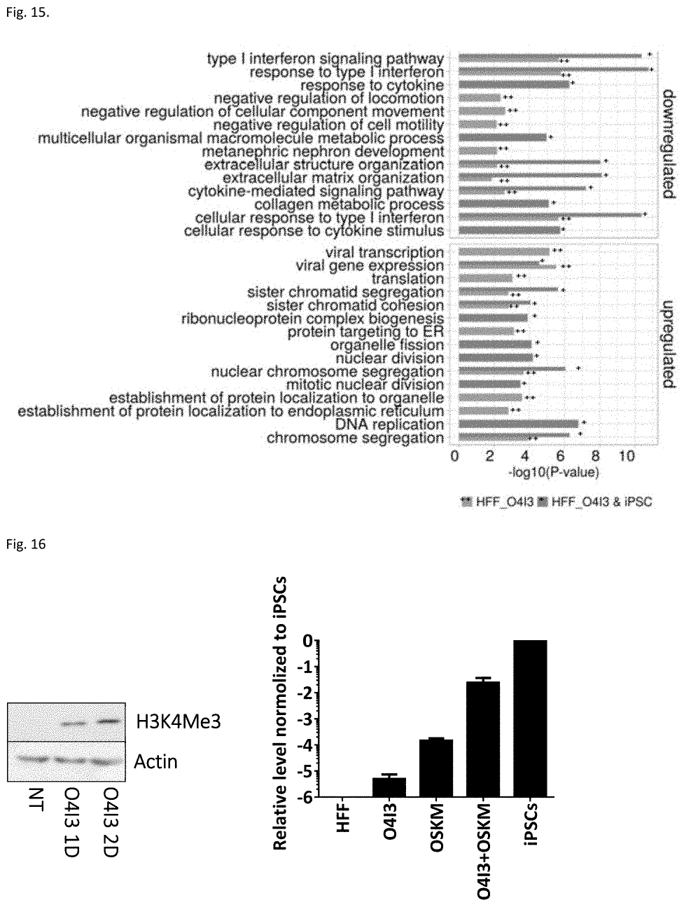

The iPSCs exhibited pluripotent characteristics and showed typical iPSC morphology (FIG. 4A). More than 95% of them co-expressed Tra-1-60 and E-Cadherin (FIG. 4C) as well as Tra-1-81 and Oct4 as analyzed by FACS (FIG. 16). Immunoblotting (FIG. 4D) and immunocytochemistry (FIG. 15) further revealed co-expression of pluripotent markers Nanog and E-Cadherin, and Oct4 and Sox2. Immunoblotting also confirmed high levels of putative iPSC markers, comparable to established pluripotent stem cells and NCCIT (FIG. 4D).

The inventors compared DNA microarray data and found that the resulting iPSCs show a distinct pattern of global gene expression to human fibroblasts, which are similar to hPSCs (FIG. 4F).

Pluripotency-associated genes (Pou5F1 encoding Oct4, CDH1 encoding E-Cadherin, Epcam and DNMT3B) were highly expressed in newly generated hiPSC_1 and hiPSC_2, whereas fibroblast-related genes (CDH2, Twist1, Twist2 and Snai2) were suppressed, comparable to characterized hPSCs (FIG. 4F). Consistently, scatter plots also show high similarity of gene expression pattern between new hiPSCs and hPSCs, but not fibroblasts (FIG. 4E) Moreover, the established iPSCs were differentiated using standard protocols and expression of markers of mesoderm (T, Slug and Snail1), Ectoderm (Pax6, Nestin and Tuj1) and Endoderm (AFP, SoxA2 and Sox17) was detected by qRT-PCR (FIG. 4B), confirming the pluripotency of our hiPSCs, which can give rise to all three germ layers.

### O4I3 Enhances the Binding Affinity of H3K4Me3 at the Promoter of Oct4

To obtain a deeper insight into the mechanism of action, global gene expression in HFF, HFF treated with O4I3 (HFF_O4I3), HFF transfected with episomal OSKM (HFF_OSKM) and hiPSCs was compared. It was found that O4I3 induced expression of genes positively regulating cell cycle progression and DNA replication (e.g. CDK1, MYC and CDC25) and promoted de-differentiation by upregulation of epithelial genes (CDH1/E-Cadherin, Epcam, Crb3 and OcIn) and downregulation of mesenchymal genes (Twist2), two essential cellular responses at the early stage of reprogramming. Of note, the overlap of up- and down-regulated genes of HFF_O4I3 and HFF_OSKM or iPSCs were more than 40% (FIG. 5A). More than 30% among them are involved in epigenetic modification and chromatin remodeling, including Oct4 (Pou5f1), Lin28, MYCN, KIFs, DNMT3B, HDAC2, TET1, EZH2 and JARID2, which are of importance in somatic reprogramming and maintenance of pluripotency.

Gene expression profiling using O4I3-treated HFF with a subset of dysregulated genes (160 microarray probes) coding for 140 proteins with protein-protein interactions involved in epigenetic modification and chromatin remodelling. The network was built with STRING using only highest confidence level interactions between the 140 proteins (and all source of information available in STRING). Expected number of interactions (edges) for random network of the same size is 242, while the observed network has 879. Average node degree 12.6 and average local clustering coefficient is 0.665.

Gene Ontology-based functional enrichment analysis of regulated genes in HFF_0413 compared to HFF was performed and found that a number of specialized terms associated with epigenetic and chromatin regulation were the top regulated terms (FIG. 15), including sister chromatid segregation (GO:0000819), sister chromatid cohesion (GO:0007062), ribonucleoprotein complex biogenesis (GO:0022613), organelle fission (GO:0048285), DNA replication (GO:0006260).

Recently, Onder et al. (Nature, 2012, 483, 598-602) screened the influence of a panel of epigenetic regulators and revealed that inhibition of DOT1L promoted iPSCs generation. The inventors analyzed the expression of those genes upon O4I3 treatment in HFFs and found that 7 genes, including DOT1L, were downregulated, which were reported by Onder et al. to enhance the reprogramming, while MBD3 was significantly upregulated, whose suppression might counteract the generation of iPSCs.

The importance of active tri-methylation of histone H3 lysine 4 (H3K4Me3) in promoting transcription factors binding to an ‘open’ chromatin state and thereby initiation of reprogramming is known in the art. In good agreement, the inventors found the abundance of H3K4Me3 in O4I3-treated reprogramming-resistant fibroblasts by immunostaining (FIG. 14) and immunoblotting (FIG. 5B). To directly study if O4I3 facilitated the binding of H3K4Me3 to the promoter of Oct4, a CHIP-qPCR experiment was performed. An increased enrichment of H3K4Me4 at the promoter of Oct4 was detected in reprogramming-resistant patient fibroblasts (FIG. 5E), which might explain the conversion of resistant fibroblasts into hiPSCs using the combination of O4I3 together with transient expression of OSKM.

### Effect on Fibroblasts Resistant to Reprogramming

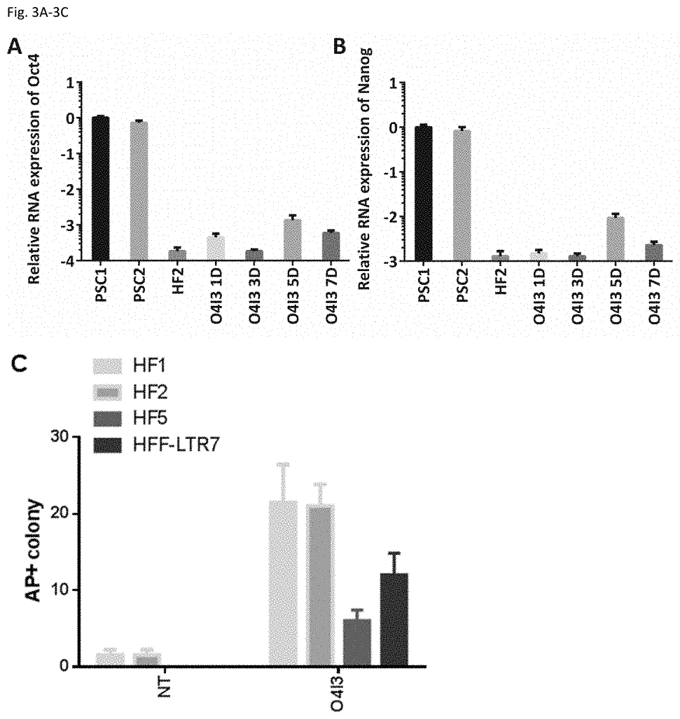

It is known in the art that senescence or other unknown factors can confer donor fibroblasts resistance to reprogramming. Therefore, O4I3 was tested for its ability to enable reprogramming of refractory fibroblasts. Two human fibroblasts were selected. HFF-LTR7-EGFP at passage >40 carrying EGFP driven by a LTR7 promoter, which was activated during reprogramming, and HF5, isolated from a patient sample, both of which were resistant to an episome-based reprogramming approach (FIG. 13). Treatment with O4I3 (500 Nm) resulted in the appearance of iPSC-like colonies appeared (FIG. 3C). Additional, Zolpidem also facilitated the reprogramming of resistant fibroblasts at 1 μM but with lower efficiency (FIG. 13).

### O4I3 Promotes the Methylation of H3K4

hiPSC derivation is an epigenetic reprogramming process. Genome-wide analysis of histone modification and chromatin remodeling revealed the number of alternations occurring at the early stage of reprogramming, including the hypermethylation of H3K4 and the demethylation of H3K27 and H3K9. These loosen the compacted heterochromatin and promote transcription factors binding to the “open” chromatin to initiate the reprogramming.

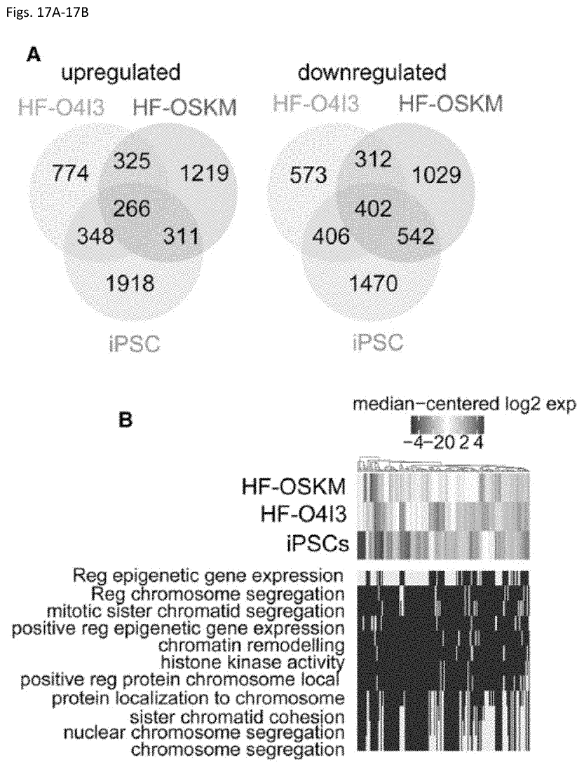

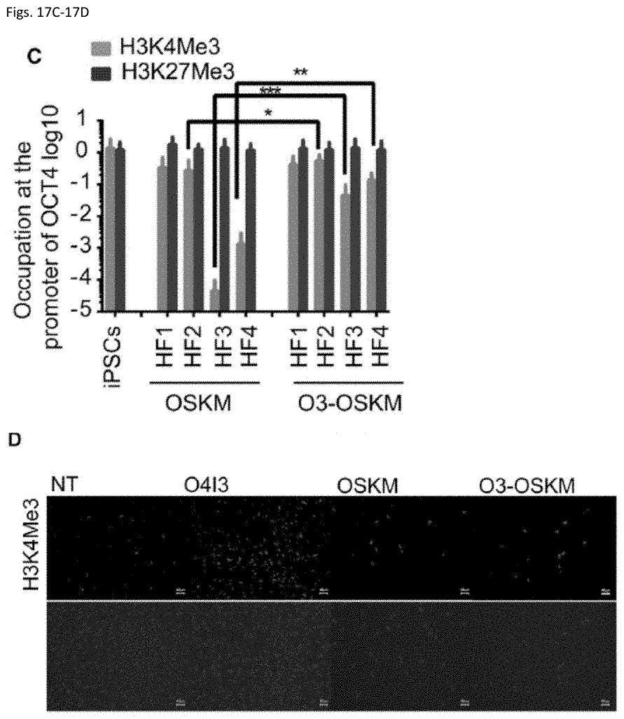

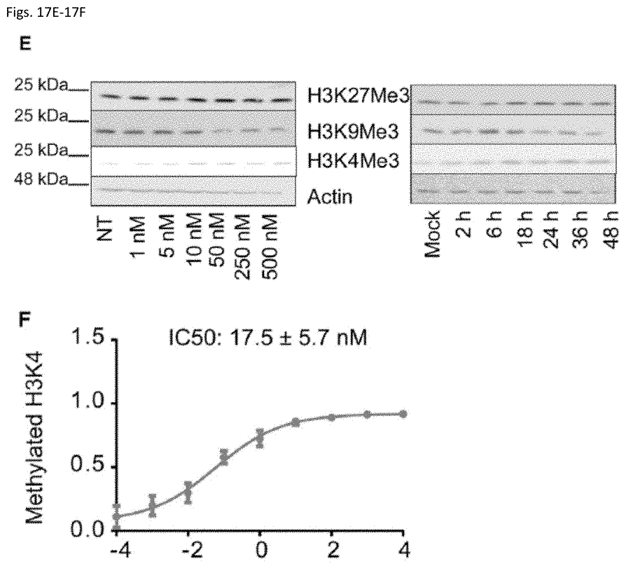

The inventors investigated the transfection efficiency in HF1 and HF4 using the same episomal vector carrying cytomegalovirus (CMV)-driven GFP. The inventors could not observe a significant difference between two cell lines, as determined by FACS analysis. This result suggested that the resistance was unlikely associated with low transfection efficiency. To study the epigenetic effects of O4I3 and its relevance to reprogramming, the inventors focused on two histone modifications at the promoter of OCT4, namely, H3K4Me3, known to be related to gene activation, and H3K27Me3, which indicates gene repression. Chromatin immunoprecipitation-qPCR results in two reprogrammable fibroblasts (HF1 and HF2) and in two reprogramming-resistant fibroblasts (HF3 and HF4) showed that OSKM was sufficient to induce abundant occupation of H3K4Me3 at the promoter of OCT4 in HF1 and HF2 in a comparable manner to those in iPSCs, while producing 1,000- to 10,000-fold less in reprogramming-resistant cells (FIG. 17C). The level of H3K27Me3 at the OCT4 promoter was minimally affected in our experiments (FIG. 17C). Analysis on the global level of H3K4Me3 by immunocytochemistry showed the increase of H3K4Me3 upon O4I3 treatment (FIG. 17D). Immunoblotting confirmed a dose- and time-dependent increase of global H3K4Me3 expression in fibroblast, whereas H3K27Me3 remained mostly unaffected (FIG. 17E). In an in vitro methylation assay, O4I3 protected methylated H3K4 with an 1050 value of 20 nM (FIG. 17F). Trimethylation of H3K9 has been reported to block reprogramming by recruiting heterochromatin protein 1 to form heterochromatin at the core of pluripotency loci, which interferes with the hypermethylation of H3K4. Accordingly, the inventors found the reduction of global H3K9Me3 posterior to H3K4Me3 activation (FIG. 17E).

### O4I3 Is a Potent KDM5 Inhibitor

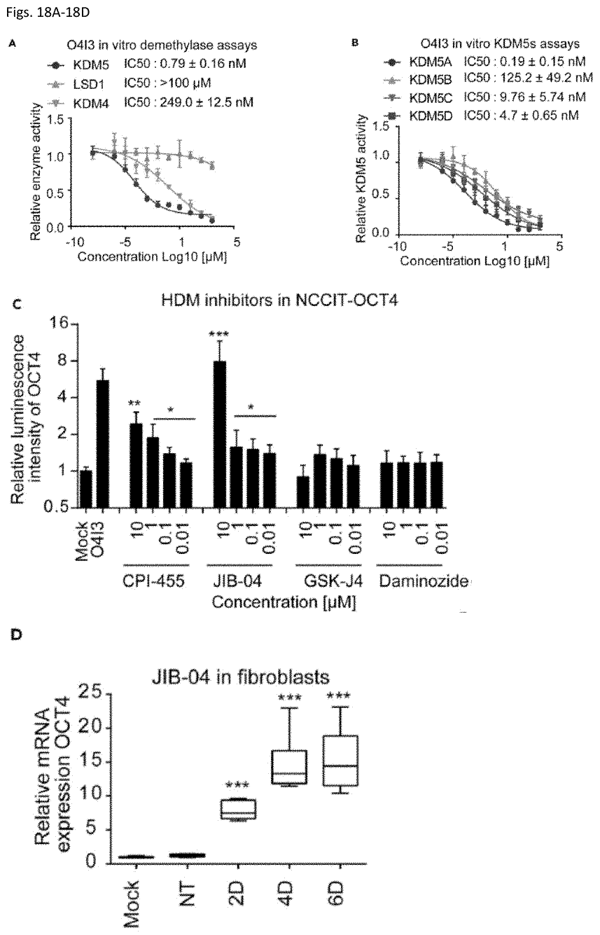

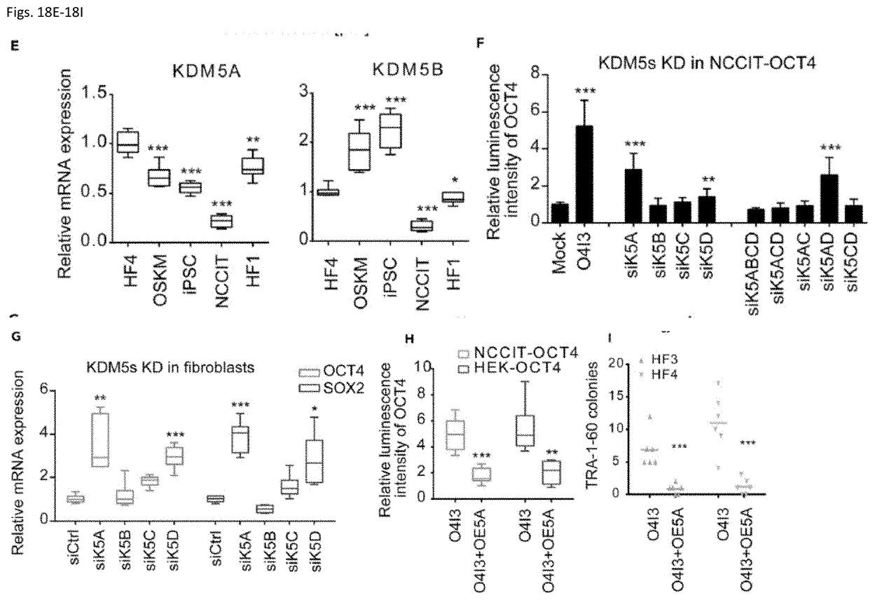

HMT and HDM are two major classes of enzymes, contributing to the regulation of histone methylation. Lysine-specific demethylase 1 (LSD1) and histone lysine demethylase 5 (KDM5, also known as JARID1) majorly catalyze demethylation of H3K4. A few KDM5 chemical inhibitors have been reported to inhibit demethylation of H3K4, leading to an increase of global methylated H3K4 in various cell types. The inventors tested the inhibitory effect of O4I3 on LSD1 and KDM5. KDM4 (also known as JMJD2), the HDM of H3K9 and H3K36, was also included. The inventors found that O4I3 inhibited KDM5 with 1050 values of 0.79 nM, whereas it inhibited KDM4 with a 500-fold less potency (1050: 249 nM). In the case of LSD1, the inventors hardly detected the inhibitory effect of the molecule even at a concentration of 100 mM (FIG. 18A).

In mammalian cells, the KDM5 family consists of four members, namely, KDM5A (known as JARID1A), KDM5B (known as JARID1B or PLU1), KDM5C (JARID1C), and KDM5D (JARID1D or SMCY). Selectivity was found for KDM5A with an IC50 value of 0.19 nM, whereas 20-, 40-, and 1,000-fold less potent 1050 values were obtained in the case of KDM5D, KDM5C, and KDM5B, respectively (FIG. 18B). As expected, zolpidem protected H3K4 methylation and inhibited the expression of both KDM5 and KDM4 atmicromolar concentrations. Next, the inventors tested four KDM chemical inhibitors. The inventors found 3- and 5-fold induction of OCT4 expression in NCCIT-OCT4 reporter cell line in the presence of KDM5 inhibitors, CPI-455 and JIB-04, respectively, but not in the presence of H3K27 demethylase inhibitor, GSK-J4 or the KDM2/7 inhibitor, daminozide, a plant growth regulator (FIG. 18C). The inventors detected the induction of OCT4, SOX2, and E-Cadherin expression in JIB-04-treated HFs (FIG. 18D).

JIB-04 is a potent KDM5A inhibitor (1050: 230 nM) and was found to produce a higher induction of OCT4 compared with the pan KDM5 inhibitor, CPI-445 (FIG. 18C). The inventors questioned if the members of the KDM5 family contribute differently to the induction of pluripotency. KDM5B has been shown to be crucial in the early embryonic development and plays an important role in ESC maintenance and neural differentiation. However, little is known about the function of other members of the KDM5 family of genes in development. The inventors thus re-analyzed the KDM5A-D expression during human preimplantation and compared their expression patterns with those of the putative pluripotency markers, OCT4 and NANOG. KDM5B showed a similar expression pattern to OCT4 and NANOG, whereas KDM5A behaved oppositely. In the case of KDM5D, the difference was not significant and KDM5C remained unaffected between ESC and oocyte. The inventors further compared the expression of KDM5A-5D in fibroblasts, reprogrammed fibroblasts by OSKM, iPSCs, and NCCIT (FIG. 18E). KDM5A was preferentially expressed in reprogramming-resistant fibroblasts, whereas KDM5B was more pluripotency related (FIG. 18E), which was in agreement with previous gene expression profiling data, as well as immunoblotting results. As JIB-04 also inhibits KDM4 and KDM6 demethylases, the inventors repressed the expression of KDM5A-D using RNA interference (small interfering RNA [siRNA]) and found significantly higher OCT4 reporter activity in NCCIT-OCT4 cells (FIG. 18F), as well as higher mRNA levels of OCT4 and SOX2 in HFs in the absence of KDM5A and KDM5D expression, but not in KDM5B and KDM5C knockdown cells (FIG. 18G). Notably, the effects of KDM5D repression were found to be less significant when compared with those of KDM5A knockdown, most probably due to the various transfection efficiencies. Moreover, knockdown of KDM5A also increased the global levels of H3K4Me3 with or without OSKM. Overexpression of KDM5A compromised these effects caused by KDM5A inhibition (either O4I3 or JIB-04) in NCCIT-OCT4 reporter cells in a concentration-dependent manner, as well as in HEK-OCT4 reporter cells (FIG. 18H). Conversely, cells were resistant to KDM5A chemical inhibitors in the absence of KDM5A expression. Of note, at concentrations of 10 and 100 nM, O4I3 treatment alone showed higher OCT4 levels when compared with that of O4I3 combined with siRNA-mediated KDM5A inhibition, which may be due to the insufficient knockdown efficiency. As expected, overexpression of KDM5A also affected the formation of TRA-1-60-positive colonies in OSKM-mediated reprogramming of HFs (FIG. 18I). Taken together, the inventors' results confirm the importance of KDM5A in the induction of pluripotency markers and the process of reprogramming of HFs.

### Inhibition of KDM5A Promotes Reprogramming of Resistant Fibroblasts

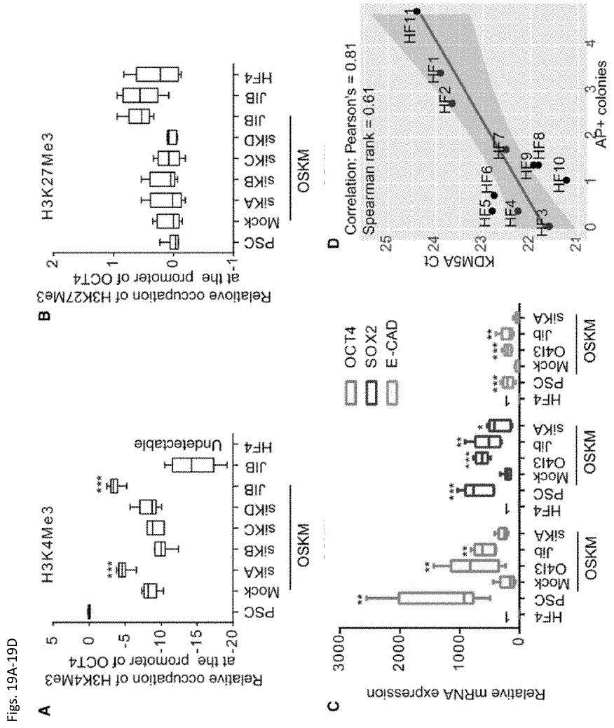

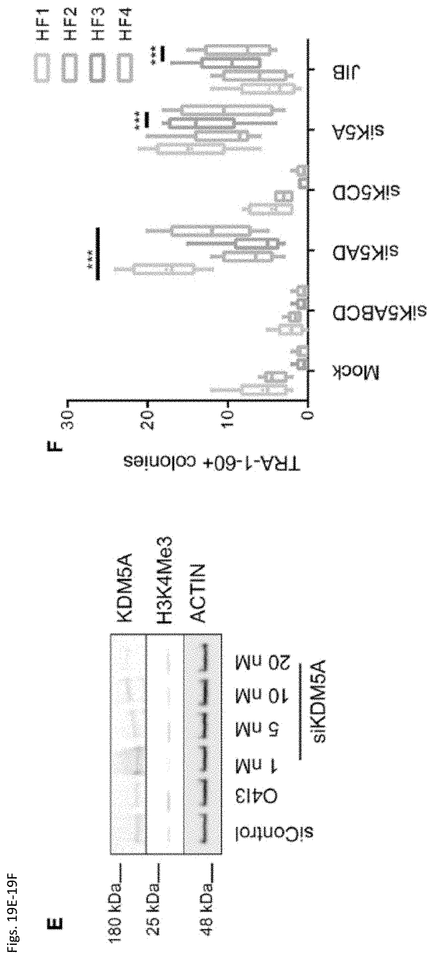

In reprogramming-resistant HF4, the level of H3K4Me3 at the promoter of OCT4 was increased by OSKM, but was still nearly 1,000-fold lower than that in PSCs (FIG. 19A). In combination with OSKM overexpression, genetic repression of KDM5A expression using either KDM5A siRNA or chemical inhibition of KDM5A activity by JIB-04, significantly elevated the levels of H3K4Me3 (FIG. 19A). However, a clear influence on H3K4 methylation was not observed in the presence of KDM5B-, KDM5C-, or KDM5D-siRNA (FIG. 19A). Correspondingly, OSKM failed to force the expression of reprogramming-associated genes (POU5F1, SOX2, and CDH1) in the absence of KDM5A siRNA or JIB-04 in reprogramming-resistant fibroblasts (FIG. 19C), implicating that OSKM was not sufficient to activate OCT4 to initiate reprogramming in those patient primary fibroblasts. Indeed, the reprogramming efficiency is negatively correlated with the expression of KDM5A in 11 HFs (FIG. 19D). Moreover, KDM5A siRNA increased the global level of H3K4Me3 in a dose-dependent manner (FIG. 19E). Of note, the alternation in the occupation of H3K27Me3 at the promoter of OCT4 was more refractory to chemical inhibition or genetic repression of KDM5s in the inventors' experiments (FIG. 19B). Transient suppression of KDM5A expression with either KDM5A siRNA or chemical inhibition mimicked the effect of O4I3 to either increase the episomal-based reprogramming efficiency in normal fibroblasts or to facilitate the formation of ESC-like TRA-1-60-positive colonies in reprogramming-resistant fibroblasts (FIG. 19F). These ESC-like colonies could be isolated and expanded to establish stable hiPSC cell lines with the expression of pluripotency markers, like OCT4, SOX2, E-Cadherin, TRA-1-60, and TRA-1-81, and full differentiation potential.

### Modifications of Lead Compound

A structure-activity relationship of the lead compound was performed to identify modifications that might further enhance the activity or exhibit other beneficial properties. The modified compounds were synthesized according to the methods disclosed herein.

### Activation of Oct4

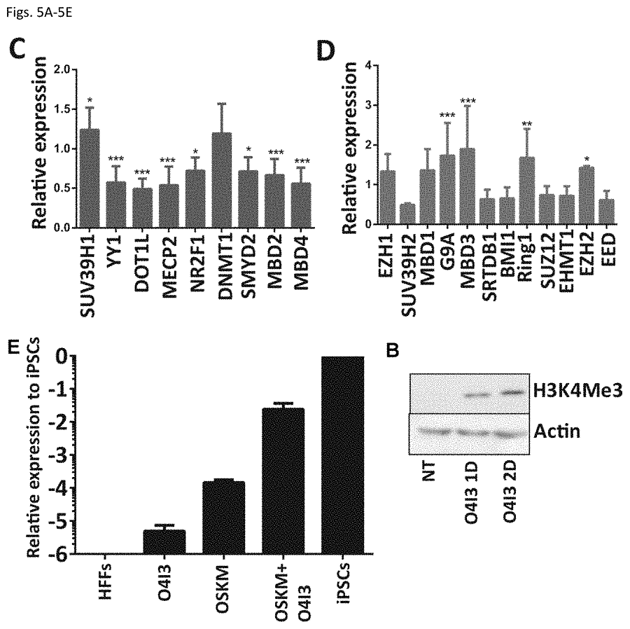

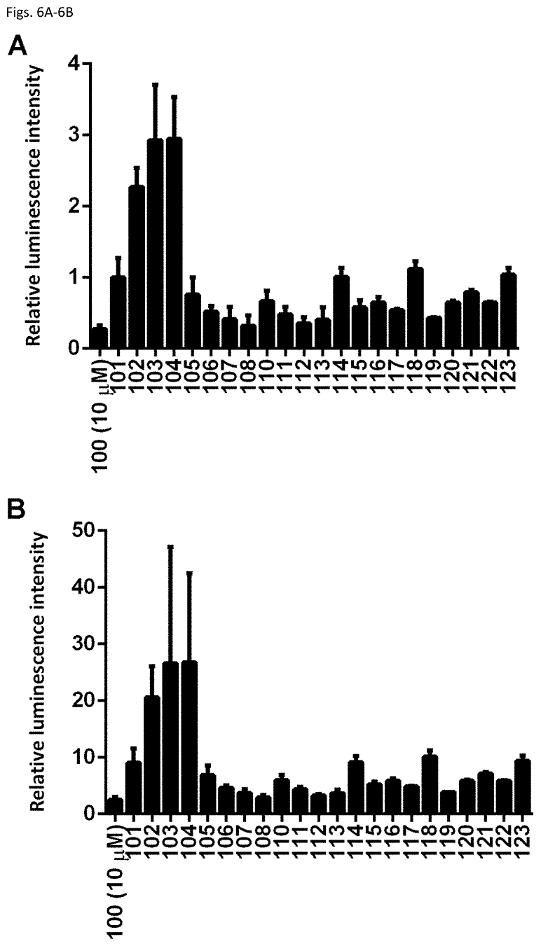

The methyl group on the aryl moiety was replaced with various substituents and their activity was evaluated in NCCIT-Oct4 cells (FIG. 6). Replacement with halogens (compounds (102), (103) and (104)) generally increased the activity (relative to compound (101); O4I3), while either electron withdrawing group, —NO₂ (compound (105)) and —CN (compound (108)), or electron donating groups, —OMe (compound (106)) and —OH (compound (107)), as substituents significantly reduced the induction at the tested concentration (100 nM) as compared to the activity of O4I3. The Oct4 inducing activity was not affected by the replacement of the methyl group with hydrogen (compound (109)).

The position of the substituents NO₂ and F was varied and resulted in a declined activity in the meta (m-) or ortho (o-) position as compared to the para position of compounds (105) and (102).

The position of the substituent was varied and a declined activity of the resulting derivatives was observed regardless of position (m- or o-) or substituent (NO₂ or F).

Further modifications on the aryl moiety as well as on the pyridine moiety were tested and their activity measured in NCCIT-Oct4 cells as shown in Table 1.

TABLE 1
Synthesized compounds tested in NCCIT-Oct4 cells

| | |
|---|---|
| 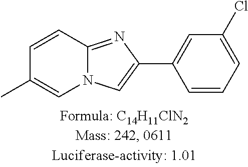 [SMILES: Cc1ccc2nc(-c3cccc(Cl)c3)cn2c1] | (114) |
| 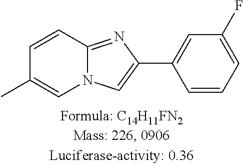 [SMILES: Cc1ccc2nc(-c3cccc(F)c3)cn2c1] | (122) |
| 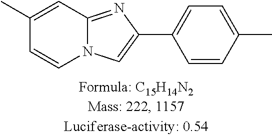 | (117) |
| 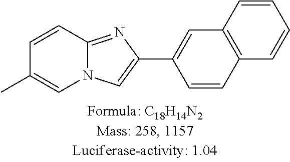 [SMILES: Cc1ccc2nc(-c3ccc4ccccc4c3)cn2c1] | (123) |
| 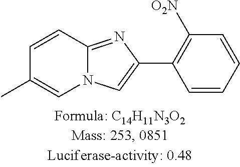 [SMILES: Cc1ccc2nc(-c3ccccc3[N+](=O)[O-])cn2c1] | (111) |
| 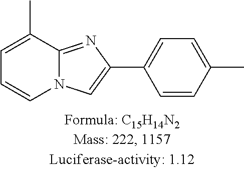 [SMILES: Cc1ccc(-c2cn3cccc(C)c3n2)cc1] | (118) |
| 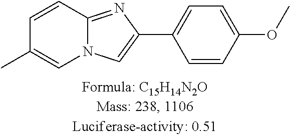 | (106) |
| 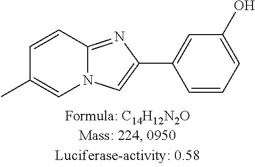 [SMILES: Cc1ccc2nc(-c3cccc(O)c3)cn2c1] | (115) |
| 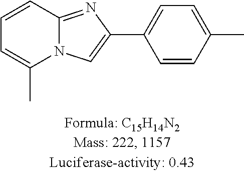 [SMILES: Cc1ccc(-c2cn3c(C)cccc3n2)cc1] | (119) |
| 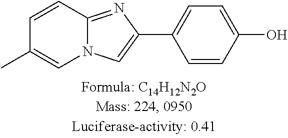 [SMILES: Cc1ccc2nc(-c3ccc(O)cc3)cn2c1] | (107) |
| 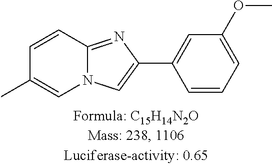 | (116) |
| 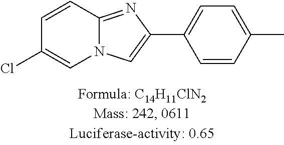 [SMILES: Cc1ccc(-c2cn3cc(Cl)ccc3n2)cc1] | (120) |
| 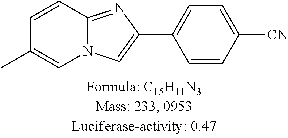 [SMILES: Cc1ccc2nc(-c3ccc(C#N)cc3)cn2c1] | (108) |
| 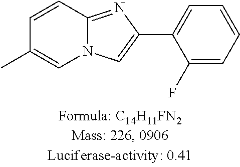 [SMILES: Cc1ccc2nc(-c3ccccc3F)cn2c1] | (113) |
| 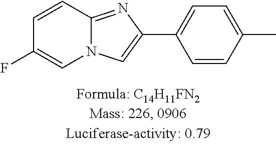 [SMILES: Cc1ccc(-c2cn3cc(F)ccc3n2)cc1] | (121) |
| 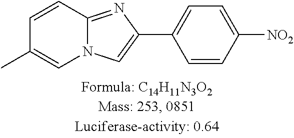 [SMILES: Cc1ccc2nc(-c3ccc([N+](=O)[O-])cc3)cn2c1] | (105) |
| 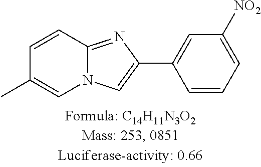 [SMILES: Cc1ccc2nc(-c3cccc([N+](=O)[O-])c3)cn2c1] | (110) |
| 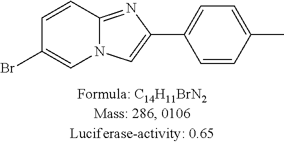 [SMILES: Cc1ccc(-c2cn3cc(Br)ccc3n2)cc1] | (122) |

Luciferase activity was measured in NCCIT-Oct4 cells and is expressed in relation to the activity of O4I3 (101).

### Chemical Optimization of O4I2 Results Metabolically Stable O4I4

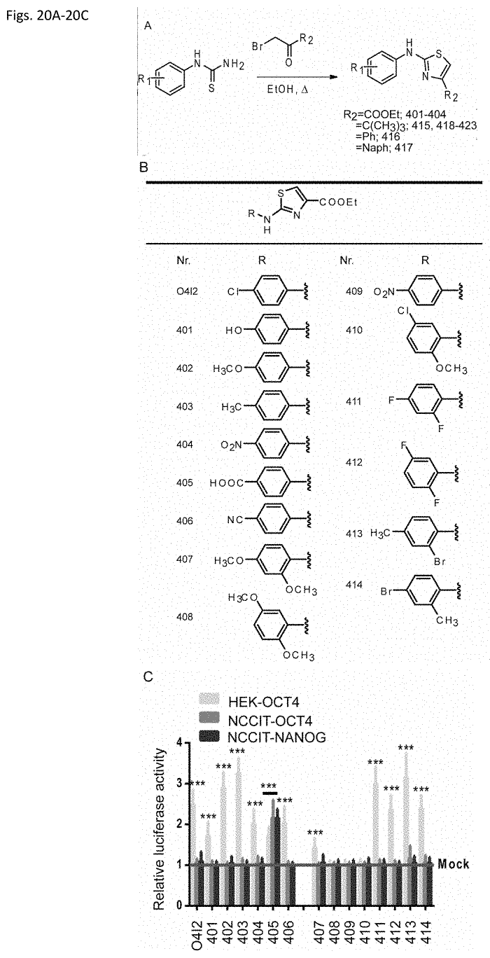

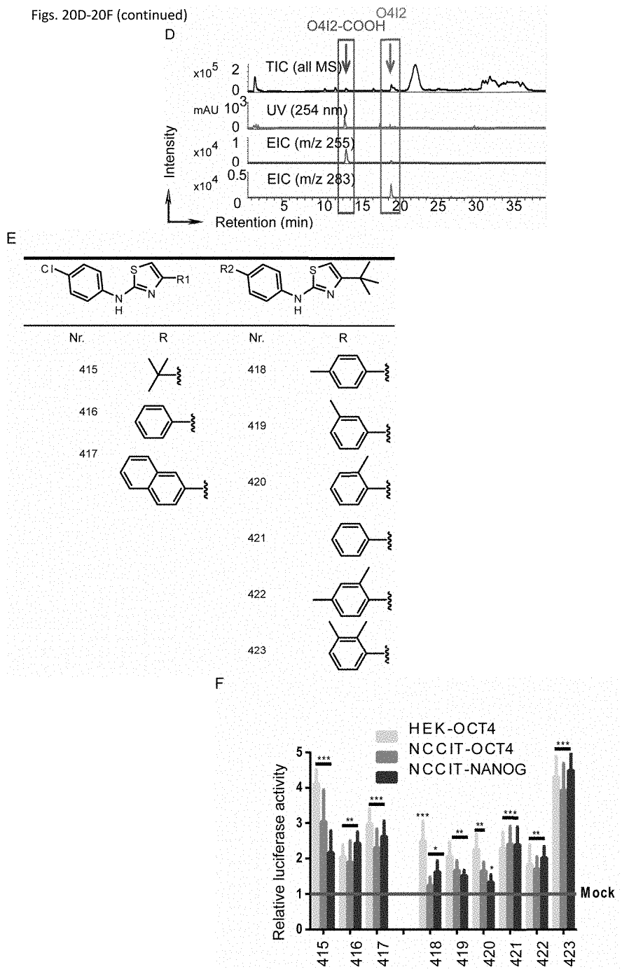

The inventors adopted Hantzsch thiazole synthesis (FIG. 20A) and produced a series of new O4I2-ester derivatives (FIG. 20B). Their activities were measured in three luciferase report cell lines, namely HEK-OCT4, driven by the activation of OCT4-promoter, and NCCIT-OCT4 and NCCIT-NANOG, whose intensities are controlled by endogenous OCT4 and NANOG respectively. Nearly all compounds showed the induction in HEK-OCT4 cells, while lost their activities in NCCIT cells (FIG. 20C). Because acid hydrolysis of O4I2 resulting 2-(4-chlorophenylamino)thiazole-4-carboxylic acid (called O4I2-COON) was inactive in the HEK-OCT4 cells, the inventors hypothesized that metabolic instability interfered the function of chemicals. Carboxylesterase 1 (CES1) is an enzyme majorly contributing to ester-to-acid conversion. The inventors compared the expression of CES1-5A using published gene expression data and found that CES1 indeed is highly expressed in NCCIT and PSCs. HPCL and LC/MS analysis on O4I2-treated cellular extraction implicated the occurrence of ester-to-acid conversion (FIG. 20D).

The inventors replaced the ethyl ester with other groups, including t-Butyl, -Ph and Naph (FIG. 20E) and detected comparable activities of all resulting compounds (415-423) in tested three luciferase reporter cell lines (FIG. 20F). Of those, 423 was among the most active compounds (FIG. 20F). The inventors called it OCT4 inducing compound 4 (O4I4) and assessed its activity further.

O4I4 Activates OCT4-Associated Signalings in Various Cells and Extends Lifespans and C. elegans Models

Consistently, in unmodified NCCIT cells O4I4 forced the expression of OCT4 (3-fold increase as compared to 0.1% DMSO treatment) and NANOG (30-fold increase) as well as other pluripotency-related genes, like E-CADHERIN, SOX2, REX1 and STAT3, 0414-mediated enrichment of OCT4 and NANOG, as well as SOX2 and E-CADHERIN, was also detected by fluorescence-activated cell sorting. Immunoblotting showed a concentration-dependent increase of OCT4 level in O4I4-treated NCCIT cells. It was reported that removal of bFGF led to the loss of pluripotency-marker expression in hPSCs, including OCT4, SOX2, E-CADHERIN and NANOG, which however was rescued in the presence of O4I4 (FIG. 21A), indicating a positive function of O4I4 in maintaining the pluripotency-associated transcriptional circuitry in hPSC.

In human primary fibroblasts, O4I4 promoted the expression of pluripotency-associated genes, including OCT4, SOX2, NANOG, LIN28, E-CADHERIN, REX1 and SALL4. A dose-dependent increase of OCT4 protein in human primary fibroblasts upon treatment was confirmed by immunoblotting (FIG. 21B). FACS analysis further confirmed the O4I4-mediated induction of OCT4 and E-CADHERIN. Based on Gene Ontology and Pathway (KEGG and REACTOME) annotations, functional enrichment analysis of differentially expressed genes in fibroblasts treated with O4I4 (20 μM) for 24 h revealed high enrichment of biological processes associated with repression of differentiation and promotion of cell proliferation and adhesion.

Recently, it was reported that transient expression of Yamanaka factors extended the lifespan in mice, indicating anti-aging effect of partial reprogramming. C. elegans provides a leading in vivo system in aging study. The inventors observed an increased survival ratio under oxidative stress (FIG. 21C) and life extension in C.elegans treated with 0.5 μM O4I4 (FIG. 21D). daf-16 encodes an ortholog of human forkhead box O transcription factor (FOXO), whose activation in nuclei is related to extend the lifespan in C.elegans. O4I4 treatment led to the nucleic translocation of darf-16 (FIG. 21E), suggesting that O4I4-mediated anti-aging effect might be due to the positive interaction of OCT4 and replated pluripotency-gene with FOXO.

O4I4 can Replace OCT4 for the Generation of Human iPSCs

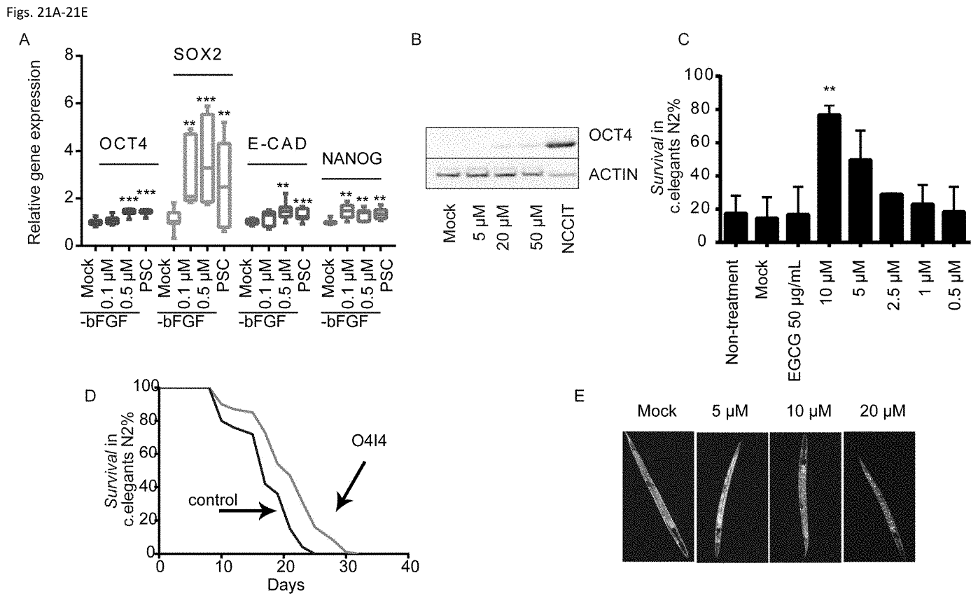

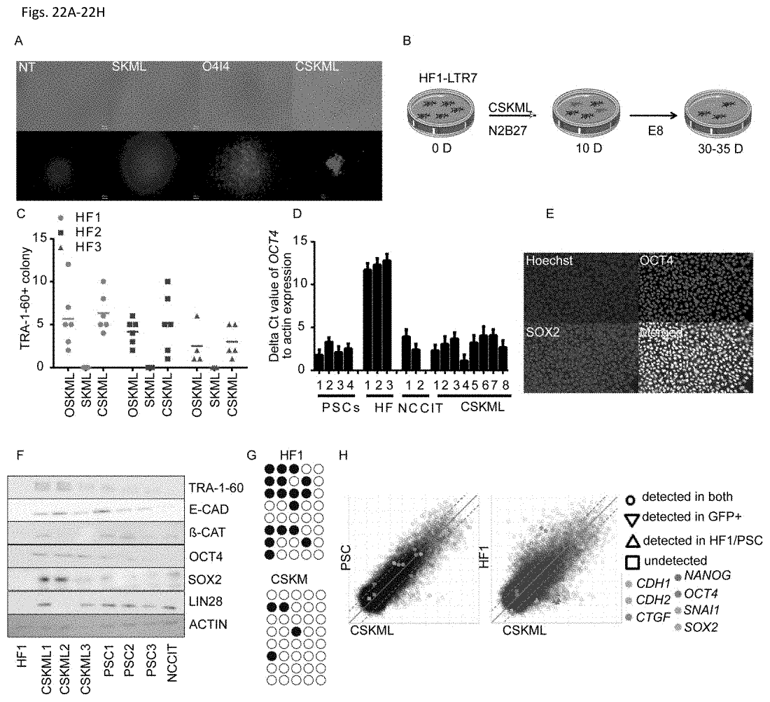

To examine if O4I4 can replace OCT4 to reprogram human fibroblasts into the pluripotent state, the inventors first introduced the pluripotent reporter pT2-LTR7-GFP into human primary fibroblasts, to generate a cell line called HF1-LTR7-GFP fibroblasts. Interestingly, some colonies with weak GFP signal appeared in fibroblasts treated merely with O4I4 (FIG. 22A). Those might be the partially reprogrammed cells (FIG. 22A), as the inventors failed to expand these colonies, implicating the lack of other factors for the generation of fully reprogrammed iPSCs. Therefore, the inventors then combined O4I4 with transient episomal expression of SOX2, KLF4, L-MYC and LIN28 (SKML) reprogram HF1-LTR7-GFP fibroblasts. ESC-like colonies appeared occasionally.

The inventors made the final combination containing O4I3, O4I4 plus SOX2, KLF4, L-MYC and LIN28, named CSKML (FIG. 22B).

In CSKML-treated fibroblasts the inventors observed a few hESC-like colonies with high GFP signal (FIG. 22A). The inventors successfully isolated and expanded the colonies reprogrammed from various primary human fibroblasts (HF1-HF3). The number of TRA-1-60 positive colonies was comparable to episomal^{OSKML}-mediated reprogramming (FIG. 22C). Some of colonies still showed typical hESC-like morphologies after expansion for long term and could be maintained in both feeder and feeder-free conditions. These cells expressed pluripotency markers, like OCT4 and SOX2, whose levels were comparable to PSCs1-4 (FIG. 22D). At the protein level, abundant expression of OCT4, SOX2, E-CADHERIN and NANOG was detected (FIG. 22E, 22F). FACS analysis confirmed that more than 95% cells were positive to OCT4, TRA-1-81, E-CADHERIN and TRA-1-60. Bisulfite sequencing demonstrated the hypomethylation at the promoter region of OCT4 expressed in CSKML-induced iPSCs (FIG. 22G) indicating reactivation of endogenous OCT4. Global gene expression profile of CSKML-iPSCs showed higher similarity to that of pluripotent stem cell lines than that of fibroblasts (FIG. 21H). To evaluate the differentiation potential of the CSKML-iPSCs, the inventors performed in vitro differentiation assays using well-established monolayer or embryonic bodies and detected the expression of genes from all three germ layers by qRT-PCR and immunocytochemistry.

Several chemical cocktails (e.g. A83-01, PD0325901, PS48, parnate, CHIR99021 and sodium butyrate) with ectopic expression of OCT4 have been reported to generate human pluripotent cells. In combination of O4I3 and O4I4 (0413/4) with those chemicals, however, the inventors could not establish fully reprogrammed iPSCs from human fibroblasts, suggesting that the further optimization of the chemical cocktail is required.

### O4I3/4 Facilitates the Chromatin Remodeling at the OCT4 Locus During Reprogramming

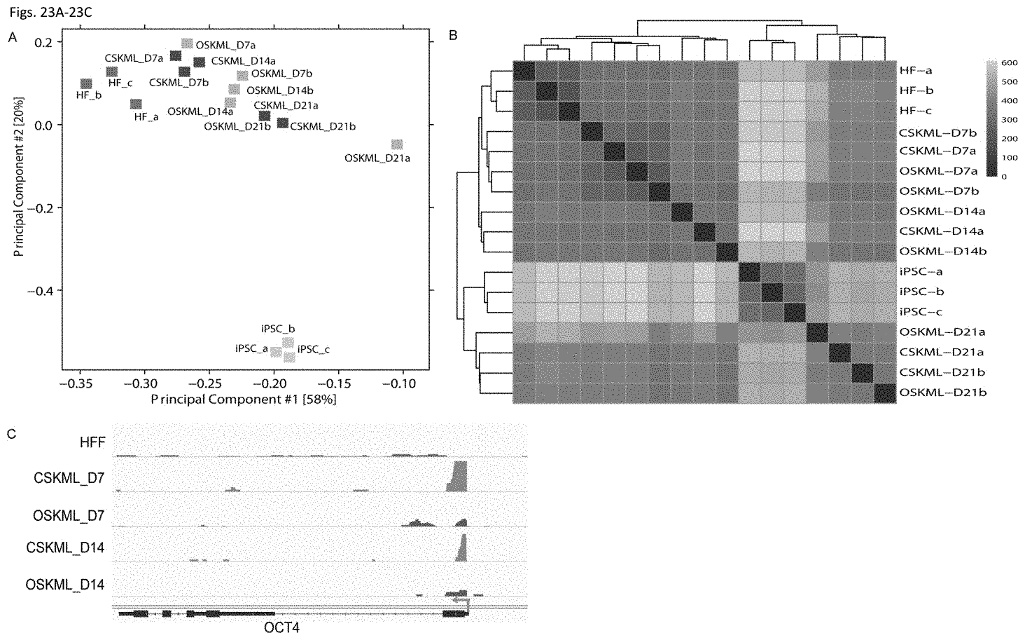

To probe the effects of O4I3/4 on chromatin remodeling during reprogramming, the inventors performed ATAC-seq on fibroblasts and TRA-1-60⁺ cells treated with either CSKML or OSKML at different time points during reprogramming. The Principal component analysis (PCA) of the genome-wide chromatin accessibility data showed that O4I3/4 dramatically promoted chromatin remodeling toward the pluripotent state similar to OSKML-mediated reprogramming (FIGS. 23A and 23B). Consistent with the transcription level, the inventors found that the chromatin at the OCT4 locus was opened more robustly in CSKML-treated fibroblasts in comparison to those in OSKML-treated fibroblasts (FIG. 23C), indicating that endogenous OCT4 was reactivated in CSKML-treated fibroblasts and the activation mechanism could be distinct from OSKML-mediated.

### HMGA1 is Involved in the Generation of CSKML

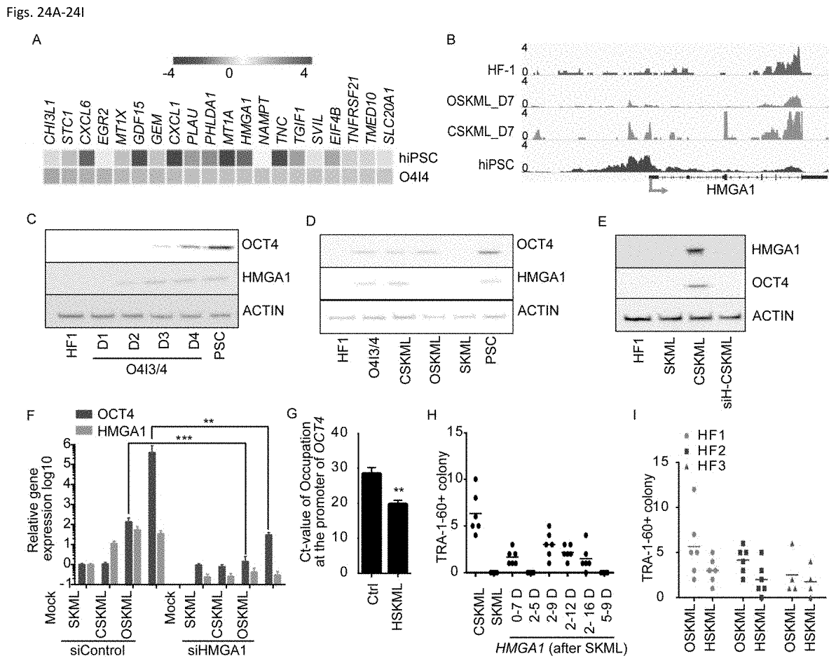

To explore how O4I3/4 reactivates endogenous OCT4 transcription to reprogram human fibroblasts into iPSCs, the inventors performed RNA-seq on human fibroblasts treated with 0413, 0414, 0413/4, CSKML and OSKML, respectively and ranked the gene up-regulated in human fibroblasts treated with O4I3/4 and compared to those in PSCs. As shown in FIG. 24A, HMGA1 (high-mobility group A1) was the top hit and has been reported to support pluripotency. Re-analysis of gene expression during the human preimplantation showed that the upregulation of HMGA1 took place as early as at 4-cell stage prior to OCT4 activation (from 8-10-cell stage), while HMGB1, a homologue of HMGA1, was kept as constant. Moreover, ATAC-seq analysis on the promoter of HMGA1 revealed higher chromatin accessibility upon CSKML treatment than OSKML (FIG. 24B), indicating that HMGA1 might involve in OCT4 activation during pluripotency establishment in vivo.

Definitely, in fibroblasts, the combination of O4I3/4 activated HMGA1 expression prior to OCT4 expression (FIGS. 24C and 24D), while a comparison level of HMGA1 in chemical-treated fibroblasts to that in PSCs was found after 4 days incubation (FIG. 24C). SKML activated neither HMGA1 nor OCT4 expression. Moreover, CSKML failed to promote OCT4 expression in HMGA1-deficient fibroblasts (FIGS. 24E and 24F), supporting the hypothesis that HMGA1 expression is essential for endogenous OCT4 activation during reprogramming.

To figure out if HMGA1 directly activates OCT4 transcription, the inventors analyzed the potential binding sites of transcription factors at the promoters of human OCT4 using Matlnspector. HMG family (indicated as SORY for SOX/SRY-sex/testis determining and related HMG box factors) is the top one in the rank list with totally matched numbers of 50. Consistent with the previous report, the occupation of HMGA1 at the promoter of OCT4 was further confirmed by ChIP-qPCR using specific HMGA1 antibody (FIG. 24G).

Finally, the inventors attempted to replace O4I4 with HMGA1 for reprogramming. The inventors overexpressed HMGA1 in human primary fibroblasts and found that transient overexpression of HMGA1 from day 2- day 9 was more efficient to reprogram human primary fibroblasts (FIG. 24H). The efficiency was slightly lower than those induced by either by CSKML or OSKML (FIGS. 24H and 24I). The inventors called iPSCs generated by overexpression of HMGA1 and SKML as HSKML (FIG. 24I). The new HSKML-iPSCs showed high expression of pluripotency markers, like OCT4, SOX2 and E-CADHERIN in various biological assesses.

Taken together, the inventors' results suggest that O4I3/4 enhanced expression of HMGA1 that contribute to endogenous OCT4 reactivation during human fibroblast reprogramming into iPSCs. Importantly, the inventors identified a novel reprograming factor HMGA1 that can replace OCT4 during human somatic cell reprograming.

### HMGA1 Expression is Dependent on BMPs Signaling Pathway

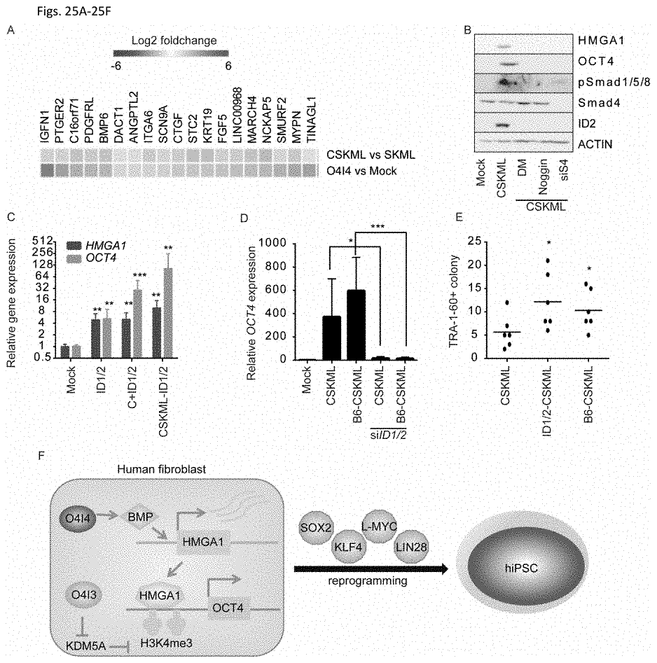

To explore if there are other signaling pathways involving in HMGA1 transcription regulation, the inventors compared the differently expressed genes in CSKML-treated and SKML-treated fibroblasts as well as O4I4-treated and DMSO-treated (mock) obtained from RNA-seq. PDGFRL, BMP6 and DACT1 were induced only in CSKML and are evident to be involved in the regulation of stemness-associated signaling pathways (FIG. 25A). Because BMP6 was reported to influence on fertility and BMPs promoted chemical- and transcription factor-mediated reprogramming, the inventors examined the role of BMP in CSKML-mediated reprogramming. BMPs activation requires phosphorylation of SMAD1/5/8, which in turn forms a complex with SMAD4 to induce expression of downstream gene, like IDs. Indeed, O4I4 promoted ID1 and ID2 expression, while ID3 was unaffected. In CSKML-treated fibroblasts phosphorylation of SMAD1/5/8 and ID1/2 expression was detected (FIG. 25B and FIG. 25C). Overexpression of IDs activated HMGA1 and slightly increased the level of OCT4, which was further elevated in the presence of chemicals or CSKML (FIG. 25C). Repressing BMP signaling in co-incubation with BMP receptor inhibitor DM (Dorsomormin), BMP antagonist protein Noggin or siRNA targeting SMAD4 inhibited the phosphorylation of SMAD1/5/8 and expression of ID1/2 (FIG. 25B), while CSKML-induced expression of HMGA1 and OCT4 also disappeared (FIG. 25B). Adding BMP6 enhanced OCT4 expression, while knockdown of ID1/2 abolished CSKML-induced either HMGA1 or OCT4 activation (FIG. 25D). An improved reprogramming efficiency was observed in the presence of BMP6 or ectopic expression of ID1/2 (FIG. 25E). Those iPSCs were characterized as abovementioned and expressed pluripotency markers including OCT4, SOX2, E-CADHERIN and TRA-1-60, Except for BMP6 BMP2 also phosphorylated SMAD1/5/and activated HMGA1 and OCT4 expression in CSKML-treated reprogramming. Interestingly, BMP9 failed to induce HMGA1 and OCT4.

### Material and Methods

### Reagents

Solvents and reagents obtained from commercial suppliers were at least of reagent grade and were distilled or dried according to prevailing methods prior to use, if necessary. For monitoring the reactions, Alugram SIL G/UV254 sheets for TLC (Macherey & Nagel) were used. Flash column chromatography was accomplished using silica gel 60 (Macherey & Nagel, 0.040-0.063 mm). The purity of compounds was determined at least more than 96% by HPLC analysis.

### General Procedure for the Synthesis

A mixture of 5-methylpyridin-2-amine and 2-bromo-1-phenylethan-1-ones (2:1) in EtOH was stirred at 60° C. for 3 h. TLC was employed to control the reaction. After removal of solvent, the solid was suspended in 1N HCl and stirred for 2-3 h, filtered and washed with water to obtain the products in good yield (>90%).

### Analytical Methods

1H and 13C NMR spectra were recorded on a Varian 300 MHz NMR system (1H: 300 MHz, 13C: 75 MHz). Chemical shifts are reported in ppm from tetramethylsilane with solvent as the internal standard (1H DMSO-d6: δ 2.50; 13C DMSO-d6: δ 39.5). The following abbreviations were used to explain the multiplicities in NMR spectra: s=singlet, d=doublet, t=triplet, q=quartet, m=multiplet. High-resolution mass spectra (HRMS) were recorded on a Bruker ApexQe hybrid 9.4 T FT-ICR (ESI).

### 6-Methyl-2-(p-tolyl)imidazo[1,2-a]pyridine (101), O4I3

O4I3 1H NMR (300 MHz, DMSO-d6) δ 8.71-8.62 (m, 2H), 7.93-7.69 (m, 4H), 7.32 (d, J=8.0 Hz, 2H), 2.41-2.24 (m, 6H). 13C NMR (75 MHz, DMSO-d6) δ 140.6, 139.1, 136.1, 135.6, 130.3, 127.5, 126.8, 126.5, 123.9, 111.6, 110.7, 21.4, 17.9. HRMS (ESI, C15 H14 N2) [M+H]+: calculated m/z, 223, 1230; found m/z, 223, 1230. IR (cm-1): 3342, 2159, 2021, 1661, 1540, 1534, 1527, 1505, 1456, 1366, 1360, 1311, 1269, 1195, 949, 848, 839, 805, 783, 767, 761, 740, 722.

### 2-(4-Fluorophenyl)-6-methyl-imidazo[1,2-a]pyridine (102)

1H NMR (300 MHz, DMSO-d6) δ 8.83-8.54 (m, 2H), 8.30-7.88 (m, 2H), 7.90-7.65 (m, 2H), 7.55-7.17 (m, 2H), 2.38 (d, J=1.1 Hz, 3H). 13C NMR (75 MHz, DMSO-d6) δ 163.4 (d, J=248.5 Hz), 139.4, 136.2, 134.9, 129.1 (d, J=8.7 Hz), 127.5, 126.9, 123.6 (d, J=3.3 Hz), 116.9 (d, J=22.1 Hz), 111.7, 111.1, 17.8. HRMS (ESI, C14 H11 F N2) [M+H]+: calculated m/z, 227, 0979; found m/z, 227.0979. IR (cm-1): 3325, 3114, 2524, 2362, 2165, 2017, 1659, 1501, 1446, 1271, 1238, 1165, 849, 823, 805, 788, 742.

### 2-(4-Chlorophenyl)-6-methyl-imidazo[1,2-a]pyridine (103)

1H NMR (300 MHz, DMSO-d6) δ 8.76 (d, J=0.6 Hz, 1H), 8.68 (q, J=1.2 Hz, 1H), 8.07-7.99 (m, 2H), 7.89-7.73 (m, 2H), 7.65-7.56 (m, 2H), 2.39 (d, J=1.1 Hz, 3H). 13C NMR (75 MHz, DMSO-d6) δ 139.5, 136.3, 135.2, 134.8, 129.8, 128.3, 127.5, 126.9, 126.0, 111.8, 111.6, 17.9. HRMS (ESI, C14 H11 Cl N2) [M+H]+: calculated m/z, 243.0684; found m/z, 243.0684. IR (cm-1): 3303, 2528, 2362, 2162, 2020, 1660, 1527, 1488, 1474, 1455, 1416, 1273, 1094, 1010, 941, 933, 843, 803, 769, 742, 729.

### 2-(4-Bromophenyl)-6-methyl-imidazo[1,2-a]pyridine (104)

1H NMR (500 MHz, DMSO-d6) δ 8.77 (d, J=0.7 Hz, 1H), 8.68 (q, J=1.3 Hz, 1H), 8.00-7.93 (m, 2H), 7.85 (d, J=9.1 Hz, 1H), 7.80-7.73 (m, 3H), 2.40 (d, J=1.2 Hz, 3H). 13C NMR (75 MHz, DMSO-d6) δ 139.6, 136.3, 135.0, 132.8, 128.6, 127.5, 126.9, 126.5, 124.0, 112.0, 111.6, 17.9. HRMS (ESI, C14 H11 Br N2) [M+H]+: calculated m/z, 287.0178; found m/z, 287.0178. IR (cm-1): 2534, 2364, 2161, 2023, 1978, 1655, 1525, 1488, 1455, 1443, 1274, 1067, 1007, 839, 804, 786, 774, 742, 712.

### 6-Methyl-2-(4-nitrophenyl)imidazo[1,2-a]pyridine (105)

1H NMR (300 MHz, DMSO-d6) δ 8.79 (s, 1H), 8.58 (d, J=1.6 Hz, 1H), 8.42-8.28 (m, 2H), 8.34-8.18 (m, 2H), 7.76 (d, J=9.2 Hz, 1H), 7.59 (d, J=9.3 Hz, 1H), 2.36 (d, J=1.1 Hz, 3H). 13C NMR (75 MHz, DMSO-d6) δ 147.8, 141.6, 136.7, 136.0, 134.3, 127.3, 126.3, 126.0, 124.8, 113.7, 113.0, 17.9. HRMS (ESI, C14 H11 N3O2) [M+H]+: calculated m/z, 254.0924; found m/z, 254.0924. IR (cm-1): 2521, 2364, 2160, 2022, 1977, 1601, 1509, 1490, 1449, 1343, 1264, 1245, 1221, 1108, 854, 834, 806, 768, 752, 741, 715.

### 2-(4-methoxyphenyl)-6-methylimidazo [1,2-a] pyridine

1H NMR: δ 8.67 (q, J=1.2 Hz, 1H), 8.63 (d, J=0.7 Hz, 1H), 8.03-7.97 (m, 2H), 7.86 (d, J=9.1 Hz, 1H), 7.77 (dd, J=9.2, 1.6 Hz, 1H), 7.15-7.09 (m, 2H), 3.83 (s, 2H), 2.50 (p, J=1.8 Hz, 3H), 2.41 (d, J=1.1 Hz, 2H). 13C NMR: δ 161.2, 139.1, 135.8, 135.8, 128.3, 127.4, 126.7, 119.2, 115.2, 111.6, 109.9, 55.9, 17.9. HRMS (ESI) calculated m/z, 239.1179; found C15H15N2O m/z, 239.1179 [M+H]+. IR (cm-1): 2539, 2436, 2366, 2157, 2028, 1975, 1660, 1617, 1594, 1577, 1561, 1507, 1441, 1367, 1294, 1256, 1186, 1160, 1023, 952, 840, 808, 795, 743.

### 4-(6-methylimidazo [1,2-a] pyridin-2-yl) phenol

1H NMR: δ 10.22 (s, 1H), 8.66 (s, 1H), 8.54 (s, 1H), 7.85 (dd, J=9.0, 2.3 Hz, 3H), 7.76 (dd, J=9.2, 1.5 Hz, 1H), 7.03-6.90 (m, 2H), 2.41 (d, J=1.2 Hz, 3H). 13C NMR δ 159.7, 139.5, 137.2, 134.9, 128.2, 126.8, 126.5, 118.3, 116.5, 112.0, 109.2, 17.9. HRMS (ESI) calculated m/z, 225.1022; found C14 H15N20 m/z, 225.1022 [M+H]+. IR (cm-1): 2489, 2158, 2027, 1975, 1617, 1591, 1506, 1459, 1397, 1375, 1292, 1275, 1246, 1219, 1179, 1112, 951, 837, 812, 766, 741.

### 4-(6-methylimidazo [1,2-a] pyridin-2-yl) benzonitrile

1H NMR: δ 8.90 (s, 1H), 8.70 (q, J=1.3 Hz, 1H), 8.29-8.18 (m, 2H), 8.09-7.98 (m, 2H), 7.88 (d, J=9.2 Hz, 1H), 7.79 (dd, J=9.3, 1.5 Hz, 1H), 2.41 (d, J=1.1 Hz, 3H). 13C NMR: δ 140.1, 136.4, 134.5, 133.6, 131.9, 127.4, 127.2, 126.9, 118.8, 113.0, 112.5, 112.2, 17.9. HRMS (ESI) calculated m/z, 234.1026; found C15H12N3 m/z, 234.1026 [M+H]+. IR (cm-1): 2781, 2702, 2536, 2498, 2446, 2368, 2156, 2025, 1974, 1656, 1613, 1557, 1528, 1498, 1446, 1302, 1267, 1163, 1135, 1039, 1021, 996, 950, 8757, 848, 835, 808, 791, 746, 717.

### 6-methyl-2-(3-nitrophenyl) imidazo [1,2-a] pyridine

1H NMR: δ 8.79 (t, J=2.0 Hz, 1H), 8.68 (s, 1H), 8.47 (q, J=1.4 Hz, 1H), 8.42 (dt, J=7.8, 1.3 Hz, 1H), 8.21 (dd, J=8.2, 1.0 Hz, 1H), 7.78 (t, J=8.0 Hz, 1H), 7.66 (d, J=9.2 Hz, 1H), 7.38 (dd, J=9.0, 1.7 Hz, 1H), 2.34 (s, 3H). 13C NMR: δ 150.31, 142.50, 137.70, 136.28, 133.14, 131.96, 131.58, 128.54, 127.49, 125.29, 121.80, 113.70, 113.01, 18.10. HRMS (ESI) calculated m/z, 254.0924; found C14H12N3O₂ m/z, 254.0924 [M+H]+. IR (cm-1): 2947, 2930, 2541, 2456, 2363, 2158, 2028, 1974, 1656, 1619, 1554, 1517, 1486, 1456, 1348, 1266, 1215, 1163, 1114, 1071, 970, 904, 879, 837, 808, 768, 724.

### 6-methyl-2-(2-nitrophenyl) imidazo [1,2-a] pyridine

1H NMR: δ 8.40-8.34 (m, 1H), 8.18 (d, J=0.7 Hz, 1H), 7.91 (dd, J=7.8, 1.4 Hz, 1H), 7.83 (dd, J=8.0, 1.2 Hz, 1H), 7.71 (td, J=7.6, 1.3 Hz, 1H), 7.56 (td, J=7.7, 1.4 Hz, 1H), 7.48 (d, J=9.1 Hz, 1H), 7.14 (dd, J=9.2, 1.7 Hz, 1H), 2.28 (d, J=1.1 Hz, 3H). 13C NMR: δ 149.2, 144.1, 140.0, 132.4, 130.9, 129.2, 129.0, 127.4, 124.9, 124.0, 122.3, 116.7, 111.1, 18.0. HRMS (ESI) calculated m/z, 254.0924; found C14H12N3O₂ m/z, 254.0924 [M+H]+. IR (cm-1): 2540, 2487, 2443, 2364, 2341, 2159, 2027, 1974, 1609, 1527, 1459, 1420, 1372, 1345, 1285, 1260, 1208, 1164, 1144, 1105, 1033, 1018, 990, 949, 854, 836, 800, 776, 761, 727.

### 2-(3-fluorophenyl)-6-methylimidazo [1,2-a] pyridine

1H NMR: δ 8.83 (s, 1H), 8.71 (q, J=1.2 Hz, 1H), 7.97 (dt, J=10.2, 2.2 Hz, 1H), 7.96-7.83 (m, 2H), 7.81 (dd, J=9.2, 1.5 Hz, 1H), 7.62 (td, J=8.1, 6.0 Hz, 1H), 7.43-7.28 (m, 1H), 2.41 (d, J=1.1 Hz, 3H). 13C NMR: δ 162.9 (d, J=244.2 Hz), 139.6, 136.4, 134.7 (d, J=2.8 Hz), 132.0 (d, J=8.5 Hz), 129.4 (d, J=8.8 Hz), 127.5, 126.9, 122.8 (d, J=2.9 Hz), 117.4 (d, J=21.1 Hz), 113.5 (d, J=24.1 Hz), 112.0, 111.9, 17.8. HRMS (ESI) calculated m/z, 227.0979; found C14H11FN2 m/z, 227.0979 [M+H]+. IR (cm-1): 3058, 3023, 3012, 2929, 2536, 2448, 2366, 2157, 2102, 2024, 1975, 1664, 1604, 1585, 1529, 1491, 1452, 1362, 1311, 1271, 1196, 1159, 1088, 799, 879, 816, 795, 746.

### 2-(2-fluorophenyl)-6-methylimidazo[1,2-a]pyridine

1H NMR: δ 8.75 (q, J=1.3 Hz, 1H), 8.64 (d, J=3.0 Hz, 1H), 8.26 (td, J=7.8, 1.7 Hz, 1H), 7.91 (d, J=9.2 Hz, 1H), 7.81 (dd, J=9.2, 1.6 Hz, 1H), 7.63-7.53 (m, 1H), 7.53-7.41 (m, 2H), 2.41 (s, 3H). 13C NMR: δ 159.6 (d, J=250.8 Hz), 139.4, 136.3, 132.5 (d, J=9.0 Hz), 130.0, 128.9, 127.2, 126.8, 125.9, 117.0 (d, J=21.4 Hz), 115.5 (d, J=11.9 Hz), 113.8 (d, J=14.0 Hz), 112.0, 18.0 (d, J=3.7 Hz). HRMS (ESI) calculated m/z, 227.0979; found C15H14FN2 m/z, 227.0979 [M+H]+. IR (cm-1): 2930, 2542, 2449, 2371, 2160, 2025, 1973, 1656, 1621, 1523, 1498, 1451, 1357, 1313, 1268, 1227, 1164, 1118, 949, 861, 805, 764, 743.

### Ethyl 2-((4-hydroxyphenyl)amino)thiazole-4-carboxylate

¹H-NMR: 1.28 (t, ³J=6.9 Hz, 3H), 4.24 (q, ³J=6.9, 2H), 6.74 (dd, ³J=6.9 Hz, ⁴J=2.1 Hz, 2H), 7.36 (dd, ³J=6.9 Hz, ⁴J=2.1 Hz, 2H), 7.63 (s, 1H), 9.12 (s, 1H), 10.00 (s, 1H). ¹³C-NMR: 14.2, 60.3, 115.5, 117.2, 119.8, 132.7, 142.4, 152.8, 160.9, 164.6. IR (cm⁻¹): 3308, 3127, 1693, 1586, 1545, 1508, 1464, 1437, 1393, 1297, 1272, 1240, 1212, 1159, 1093, 1021, 959, 876, 840, 819, 793, 771, 722, 674, HRMS (ESI) Calculated m/z: 265.0641; found m/z: 265.0641 [M+H]⁺. Anal. Calcd for C12H12N2O₃S: C, 54.53; H, 4.58; N, 10.60. Found: C, 54.91; H, 4.67; N, 10.23.

### Ethyl 2-((4-methoxyphenyl)amino)thiazole-4-carboxylate

¹H-NMR: 1.28 (t, ³J=7.2 Hz, 3H), 3.72 (S, 3H), 4.25 (q, ³J=7.2 Hz, 2H), 6.92 (dd, ³J=6.9 Hz, ⁴J=2.1 Hz, 2H), 7.52 (dd, ³J=6.9 Hz, ⁴J=2.4 Hz, 2H), 7.67 (s, 1H), 10.23 (s, 1H). ¹³C-NMR spectrum: 14.2, 55.3, 60.4, 114.3, 117.7, 119.3, 134.1, 142.0, 154.6, 160.8, 164.1. IR (cm⁻¹): 3019, 2833, 2541, 2159, 2031, 1976, 1714, 1635, 1588, 1535, 1510, 1323, 1267, 1253, 1212, 1182, 1125, 1109, 1035, 1017, 822, 801, 767, 661, HRMS (ESI) Calculated m/z: 279.0798; found m/z: 279.0798 [M+H]+. Anal. Calcd for C₁₃H₁₄N₂O₃S: C, 56.10; H, 5.07; N, 10.07. Found: C, 56.18; H, 5.47; N, 9.82.

### Ethyl 2-(p-tolylamino)thiazole-4-carboxylate

¹H-NMR: 1.27 (t, ³J=6.9, 3H), 2.23 (s, 3H), 4.23 (q, ³J=6.9 Hz, 2H), 7.11 (d, ³J=8.1 Hz, 2H), 7.47 (d, ³J=8.7 Hz, 2H), 7.69 (s, 1H), 10.25 (s, 1H). ¹³C-NMR: 14.7, 20.8, 60.8, 117.7, 118.4, 129.9, 131.1, 138.8, 142.8, 161.3, 163.9. IR (cm⁻¹): 3262, 2509, 2158, 2023, 1977, 1732, 1542, 1508, 1438, 1190, 1188, 1026, 795, 704, 675. HRMS (ESI) Calculated m/z: 263.0849; found m/z: 263.0849 [M+H]⁺. Anal. Calcd for C₁₃H₁₄N₂O₂S: C, 59.52; H, 5.38; N, 10.68. Found: C, 59.32; H, 5.66; N, 10.51.

### Ethyl 2((4-nitrophenyl)amino)thiazole-4-carboxylate

¹H-NMR: 1.31 (t, ³J=7.2 Hz, 3H), 4.29 (q, ³J=6.9 Hz, 2H), 7.83-7.86 (m, 2H), 7.95 (s, 1H), 8.23-8.26 (m, 2H), 11.16 (s, 1H). ¹³C-NMR: 14.2, 60.6, 116.3, 120.7, 125.5, 140.5, 142.4, 146.4, 160.5, 161.8. IR (cm⁻¹): 3316, 3114, 2440, 2159, 2031, 1976, 1688, 1594, 1561, 1527, 1510, 1487, 1419, 1323, 1303, 1254, 1234, 1181, 1112, 1093, 1027, 977, 882, 859, 847, 822, 772, 749, 689, HRMS (ESI) Calculated m/z: 294.0543; found m/z: 294.0543 [M+H]⁺. Anal. Calcd for C₁₂H₁₁N3O₄S: C, 49.14; H, 3.78; N, 14.33. Found: C, 49.18; H, 3.47; N, 14.42.

### 4-((4-(Ethoxycarbonyl)thiazol-2-yl)amino)benzoic acid

¹H-NMR: 1.28 (t, ³J=8.1 Hz), 4.24 (q, ³J=6.9 Hz, 2H), 7.75 (d, ³J=9.0 Hz, 2H), 7.87 (s, 1H), 7.93 (d, ³J=9.0 Hz, 2H), 10.82 (s, 1H), 11.17 (s, 1H). ¹³C-NMR: 14.2, 60.2, 116.1, 119.7, 122.3, 130.6, 142.4, 144.7, 160.6, 162.2, 165.3. IR (cm⁻¹): 3640, 3261, 2924, 2520, 2163, 2023, 1722, 1674, 1595, 1548, 1439, 1372, 1322, 1277, 1179, 1121, 1099, 1020, 848, 764, 715. HRMS (ESI) Calculated m/z: 315,0410; found m/z: 315,0410 [M+Na]⁺. Anal. Calcd for C₁₃H₁₂N₂O₄S C, 53.42; H, 4.14; N, 9.58. Found: C, 53.63; H, 4.24; N, 9.38.

### Ethyl 2-((4-cyanophenyl)amino)thiazole-4-carboxylate

¹H-NMR: 1.30 (t, ³J=6.9 Hz, 3H), 4.28 (d, ³J=6.9 Hz, 2H), 7.76-7.83 (m, 4H), 7.90 (s, 1H), 10.92 (s, 1H), ¹³C-NMR: 14.2, 60.6, 102.7, 116.8, 120.0, 121.9, 133.5 142.4, 144.4, 160.5, 162.0. IR(cm⁻¹): 3631, 3304, 3190, 3120, 2514, 2212, 2162, 2023, 1975, 1694, 1601, 1524, 1448, 1411, 1316, 1216, 1174, 1121, 1088, 1019, 828, 785, 720. HRMS (ESI) Calculated m/z: 296.0464; found m/z: 296.0464 [M+Na]⁺. Anal. Calcd for C₁₃H₁₁N3O2S C, 57.13; H, 4.06; N, 15.37. Found: C, 57.18; H, 4.07; N, 15.43.

### Ethyl 2-((2, 4-dimethoxyphenyl)amino)thiazole-4-carboxylate

¹H-NMR: 1.27 (t, ³J=6.9 Hz, 3H), 3.76 (s, 3H), 3.81 (s, 3H), 4.24 (q, ³J=7.2 Hz, 2H), 6.51-6.55 (m, 1H), 6.64-6.65 (m, 1H), 7.62 (s, 1H), 7.83-7.86 (m, 1H), 9.56 (s, 1H). ³C-NMR: 14.2, 55.4, 55.7, 60.4, 99.3, 104.4, 118.0, 122.5, 122.6, 141.1, 151.5, 156.8, 160.6, 166.2. IR (cm⁻¹): 3053, 2974, 2522, 2159, 2031, 1977, 1729, 1618, 1586, 1518, 1460, 1334, 1257, 1213, 1197, 1179, 1156, 1129, 1097, 1032, 1011, 836, 818, 768, 666. HRMS (ESI) Calculated m/z: 309.0904; found m/z: 309.0904 [M+H]⁺. Anal. Calcd for C₁₄H₁₆N₂O₄S C, 54.53; H, 5.23; N, 9.09. Found: C, 54.42; H, 5.47; N, 9.01.

### Ethyl 2-((2, 5-dimethoxyphenyl)amino)thiazole-4-carboxylate

¹H-NMR: 1.29 (t, ³J=6.9 Hz, 3H), 3.72 (s, 3H), 3.80 (s, 3H), 4.25 (q, ³J=7.2 Hz, 2H), 6.49-6.53 (m, 1H), 6.91-6.94 (m, 1H), 7.75 (s, 1H), 8.25-8.26 (m, 1H), 9.79 (s, 1H). ¹³C-NMR: 14.2, 55.1, 56.3, 60.3, 105.1, 105.7, 111.4, 130.6, 141.8, 142.0, 142.1, 153.3, 160.8. 163.2. IR (cm⁻¹): 3321, 3119, 2520, 2159, 2031, 1977, 1687, 1602, 1541, 1518, 1481, 1462, 1449, 1368, 1331, 1280, 1251, 1216, 1206, 1173, 1125, 1088, 1052, 1020, 977, 957, 895, 876, 860, 786, 761, 751, 711, 654. HRMS (ESI) Calculated m/z: 309.0904; found m/z: 309.0904 [M+H]⁺. Anal. Calcd for C₁₄H₁₆N₂O₄S: C, 54.53; H, 5.23; N, 9.09. Found: C, 54.43; H, 5.48; N, 9.11.

### Ethyl 2-((3, 5-bis(trifluoromethyl)phenyl)amino)thiazole-4-carboxylate

¹H-NMR: 1.31(t, ³J=7.2 Hz, 3H), 4.27 (d, ³J=7.2 Hz, 2H), 7.61 (s, 1H), 7.93 (m, 2H), 8.38 (s, 1H), 11.10 (s, 1H), ¹³C-NMR: 14.4, 61.0, 113.6-114.5, 116.9 (d, J=4.3 Hz), 120.5, 123.7 (d, J=272.8 Hz), 127.0, 134.2 (q, J=32.6 Hz), 142.7 (d, J=1.7 Hz), 161.0, 162.4. IR (cm⁻¹): 3642, 3315, 2510, 2162, 2023, 1975, 1682, 1578, 1536, 1377, 1269, 1114, 1011, 936, 874, 691, HRMS (ESI) Calculated m/z: 407.0259; found m/z: 407.0259 [M+Na]t Anal. Calcd for C₁₄H₁₀F₆N₂O₂S.0.25 H₂O C, 43.25; H, 2.72; N, 7.21. Found: C, 43.46; H, 2.80; N, 6.95.

### Ethyl 2-((5-chloro-2-methoxyphenyl)amino)thiazole-4-carboxylate

¹H-NMR: 1.30 (t, ³J=7.2 Hz, 3H), 3.87 (s, 3H), 4.26 (q, ³J=7.2 Hz, 2H), 6.99-7.01 (m, 2H), 7.80 (s, 1H), 8.67 (d, ³J=2.1 Hz, 1H), 9.97 (s, 1H). ¹³C-NMR: 14.2, 56.0, 60.4, 111.9, 117.0, 119.9, 120.9, 124.3, 131.0, 141.8, 146.4, 160.7, 162.8. IR (cm⁻¹): 3315, 3122, 2500, 2159, 2031, 1976, 1686, 1596, 1536, 1509, 1491, 1416, 1331, 1250, 1212, 1173, 1125, 1021, 876, 798, 786, 761, 752. HRMS (ESI) Calculated m/z: 313.0408; found m/z: 313.0408 [M+H]⁺. Anal. Calcd for C₁₃H₁₃ClN₂O₃S.0.5H₂O: C, 48.53; H, 4.39; N, 8.71. Found: C, 48.93; H, 4.35; N, 8.37.

### Ethyl 2-((2, 4-difluorophenyl)amino)thiazole-4-carboxylate

¹H-NMR: 1.28 (t, ³J=6.9 Hz, 3H), 4.25 (q, ³J=7.2 Hz, 2H), 7.11-7.14 (m, 1H), 7.23-7.36 (m, 1H), 7.78 (s, 1H), 8.36-8.40 (m, 1H), 10.13 (s, 1H). ¹³C-NMR: ¹³C-NMR: 14.6, 60.9, 106.3 (d, J=2.1 Hz), 107.9 (dd, J=24.6, 7.5 Hz), 116.2 (dd, J=21.5, 10.2 Hz), 120.8, 130.2 (m), 142.3, 147.9 (dd, J=239.1, 2.4 Hz), 158.5 (dd, J=237.3, 2.0 Hz), 161.7, 162.8. IR (cm⁻¹): 3246, 2928, 2518, 2159, 2031, 1976, 1716, 1702, 1538, 1511, 1456, 1432, 1369, 1333, 1318, 1257, 1226, 1197, 1172, 1142, 1116, 1092, 1020, 971, 957, 848, 839, 810, 786, 768, 742, 730, 719, 670, HRMS (ESI) Calculated m/z: 285.0504; found m/z: 285.0504 [M+H]⁺. Anal. Calcd for C₁₂H₁₀F₂ N₂O₂S.0.67H₂O: C, 49.65; H, 3.70; N, 9.65. Found: C, 49.65; H, 3.46; N, 9.22.

### Ethyl 2-((2, 5-difluorophenyl)amino)thiazole-4-carboxylate

¹H-NMR: 1.30 (t, ³J=6.9 Hz, 3H), 4.27 (q, ³J=7.2 Hz, 2H), 6.78-6.84 (m, 1H), 7.25-7.33 (m, 1H), 7.86 (s, 1H), 8.46-8.53 (m, 1H), 10.45 (s, 1H). ¹³C-NMR: ¹³C-NMR: 14.6, 60.8, 104.5 (dd, J=27.1, 23.4 Hz), 111.6 (dd, J=21.7, 3.6 Hz), 120.0, 121.8 (dd, J=9.1, 2.8 Hz), 125.8 (dd, J=11.1, 3.4 Hz), 142.4, 152.4 (dd, J=247.2, 12.2 Hz), 157.3 (dd, J=241.8, 11.3 Hz), 161.1, 164.1. IR (cm⁻¹): 3308, 3124, 2517, 2159, 2031, 1976, 1702, 1644, 1551, 1524, 1491, 1483, 1444, 1373, 1331, 1241, 1214, 1194, 1182, 1116, 1093, 1027, 967, 876, 865, 798, 780, 743, 723, HRMS (ESI) Calculated m/z: 307.0323; found m/z: 307.0323 [M+Na]+. Anal. Calcd for C₁₂H₁₀F₂ N₂O₂S.0.67H₂O: C, 49.65; H, 3.70; N, 9.65. Found: C, 49.44; H, 3.91; N, 9.35.

### Ethyl 2((2-bromo-4-methylphenyl)amino)thiazole-4-carboxylate

¹H-NMR: 1.27 (t, ³J=6.9 Hz, 3H), 2.29 (s, 3H), 4.23 (q, ³J=7.2 Hz, 2H), 7.19-7.20 (m, 1H), 7.22-7.23 (m, 1H), 7.71 (s, 1H), 7.81-7.84 (m, 1H), 9.63 (s, 1H). ¹³C-NMR: 14.2, 20.0, 60.3, 115.9, 1!8.9, 124.1, 129.1, 133.2, 135.7, 136.1, 142.1, 160.8, 165.5. IR (cm⁻¹): 3630, 3121, 2982, 2925, 2517, 2162, 2024, 1975, 1718, 1549, 1485, 1384, 1203, 1090, 1035, 800, 728, 666. HRMS (ESI) Calculated m/z: 340.9954; found m/z: 340.9954 [M+H]⁺. Anal. Calcd for C₁₃H₁₃BrN₂O₂S.0.5 H₂O: C, 44.58; H, 4.03; N, 8.00. Found: C, 44.59; H, 3.63; N, 7.61.

### Ethyl 2((4-bromo-2-methylphenyl)amino)thiazole-4-carboxylate

¹H-NMR: 1.27 (t, ³J=8.4 Hz, 3H), 2.26 (s, 3H), 4.24 (q, ³J=7.2 Hz, 2H), 7.36-7.43 (m, 2H), 7.73 (s, 1H), 7.90-7.93 (m, 1H), 9.55 (s, 1H). ¹³C-NMR: 14.2, 17.7, 60.4, 115.0, 118.8, 122.5, 129.2, 131.4, 132.9, 138.4, 142.1, 160.8, 164.8. IR (cm⁻¹): 3128, 2984, 2929, 2517, 2162, 2023, 1975, 1725, 1548, 1479, 1387, 1207, 1089, 1024, 876, 792, 711. HRMS (ESI) Calculated m/z: 340.9954; found m/z: 340.9954 [M+H]⁺. Anal. Calcd for C₁₃H₁₃BrN₂O₂S.0.5 H₂O: C, 44.58; H, 4.03; N, 8.00. Found: C, 44.80; H, 3.81; N, 7.66.

### 4-(Tert-butyl)-N-(4-chlorophenyl)thiazol-2-amine

¹H-NMR: 1.27 (s, 9H), 6.44 (s, 1H), 7.33 (dd, ³J=2.1 Hz, ⁴J=6.9 Hz, 2H), 7.65 (dd, ³J=2.1 Hz, ⁴J=6.9 Hz, 2H), 10.20 (s, 1H). ¹³C-NMR: 14.7, 20.8, 117.7, 118.4, 129.9, 138.8, 142.8, 161.3, 163.9. IR (cm⁻¹): 3147, 2923, 2853, 2511, 2030, 1976, 1561, 1529, 1488, 1308, 1218, 1201, 1100, 1091, 1014, 954, 854, 827, 801, 713, 692. HRMS (ESI) Calculated m/z: 267.0717; found m/z: 267.0717 [M+H]⁺. Anal. Calcd for C₁₃H₁₅ClN₂S: C, 58.53; H, 5.67; N, 10.50. Found: C, 58.20; H, 5.77; N, 10.45.

### N-(4-chlorophenyl)-4-phenylthiazol-2-amine

¹H-NMR: 7.31-7.46 (m, 6H), 7.77 (d, ³J=8.7 Hz, 2H), 7.92 (d, ³J=7.2 Hz, 2H), 10.41 (s, 1H). ¹³C-NMR: 103.3, 118.2, 124.4, 125.7, 127.6, 128.6, 128.8, 134.4, 140.0, 150.1, 162.7. IR (cm⁻¹): 3378, 3112, 2509, 2159, 2031, 1976, 1585, 1556, 1481, 1442, 1416, 1386, 1323, 1301, 1278, 1215, 1199, 1172, 1090, 1071, 1055, 1025, 1011, 915, 843, 831, 919, 770, 687, 671. HRMS (ESI) Calculated m/z: 287.0404; found m/z: 287.0404 [M+H]⁺. Anal. Calcd for C₁₅H₁₁ClN₂S: C, 62.84; H, 3.87; N, 9.77. Found: C, 62.91; H, 3.72; N, 9.85.

### N-(4-chlorophenyl)-4-(naphthalen-2-yl)thiazol-2-amine

¹H-NMR: 7.41-7.45 (m, 2H), 7.48-7.56 (m, 3H), 7.81-7.85 (m, 2H), 7.90-8.08 (m, 4H), 8.46 (s, 1H), 10.49 (s, 1H). ¹³C-NMR: 104.2, 118.4, 124.0, 124.3, 124.5, 126.0, 126.4, 127.6, 128.1, 128.2, 128.9, 131.8, 132.5, 133.2, 140.1, 150.0, 162.8. IR (cm⁻¹): 3624, 2965, 2521, 21331, 1976, 1615, 1583, 1571, 1530, 1491, 1097, 815, 806, 770, 743. HRMS (ESI) Calculated m/z: 337.0561; found m/z: 337.0561 [M+H]⁺. Anal. Calcd for C₁₉H₁₃ClN₂S: C, 67.75; H, 3.89; N, 8.32. Found: C, 67.51; H, 3.99; N, 8.24.

### 4-(^{tert}-Butyl)-N-(p-tolyl)thiazol-2-amine

¹H-NMR: 1.26 (s, 9H), 2.23 (s, 3H), 6.35 (s, 1H), 7.09 (d, ³J=6.9 Hz, 2H), 7.47 (d, ³J=6.4 Hz, 2H), 9.91 (s, 1H). ¹³C-NMR: 20.3, 29.6, 34.3, 98.7, 116.8, 129.3, 129.7, 139.1, 161.5, 162.8. HRMS (ESI) Calculated m/z: 247.1263; found m/z: 247.1263 [M+H]⁺. Anal. Calcd for C₁₄H₁₈N2S: C, 68.25; H, 7.36; N, 11.37. Found: C, 68.01; H, 7.35; N, 11.30.

### 4-(^{tert}-Butyl)-N-(m-tolyl)thiazol-2-amine

¹H-NMR: 1.26 (s, 9H), 2.27 (s, 3H), 6.38 (s, 1H), 6.71-6.75 (m, 1H), 7.16(t, ³J=7.7 Hz, 1H), 7.39-7.43 (m, 2H), 9.95 (s, 1H). HRMS (ESI) Calculated m/z: 247.1263; found m/z: 247.1263 [M+H]⁺. Anal. Calcd for C₁₄H₁₈N₂S: C, 68.25; H, 7.36; N, 11.37. Found: C, 68.07; H, 7.21; N, 11.35.

### 4-(^{tert}-Butyl)-N-(o-tolyl)thiazol-2-amine

¹H-NMR: 1.23 (s, 9H), 2.20 (s, 3H), 6.25 (s, 1H), 6.96 (dd, ³J=8.1 Hz, ⁴J=2.1 Hz, 1H), 7.00 (s, 1H), 7.23 (d, ³J=8.1 Hz, 1H), 7.63 (s, 1H), 9.03 (s, 1H). ¹³C-NMR: 18.1, 29.6, 34.3, 99.1, 120.5, 123.0, 126.4, 128.5, 130.5, 139.7, 161.3, 164.8. HRMS (ESI) Calculated m/z: 247.1263; found m/z: 247.1263 [M+H]⁺. Anal. Calcd for C₁₄H₁₈N₂S: C, 68.25; H, 7.36; N, 11.37. Found: C, 68.13; H, 7.31; N, 11.25.

### 4-(^{tert}-Butyl)-N-phenylthiazol-2-amine

¹H-NMR: 1.27 (s, 9H), 6.40 (s, 1H), 6.91 (dt, ³J=7.4 Hz, ⁴J=1.1 Hz, 1H), 7.26-7.32 (m, 2H), 7.59-7.63 (m, 2H), 10.04 (s, 1H). ¹³C-NMR: 29.6, 34.4, 99.1, 116.5, 120.8, 128.7, 141.5, 161.5, 162.5. HRMS (ESI) Calculated m/z: 233.1107; found m/z: 233.1107 [M+H]⁺. Anal. Calcd for C₁₃H₁₆N₂S: C, 67.20; H, 6.94; N, 12.06. Found: C, 67.10; H, 6.92; N, 12.25.

### 4-(^{tert}-Butyl)-N-(2,4-dimethylphenyl)thiazol-2-amine

¹H-NMR: 1.23 (s, 9H), 2.20 (s, 3H), 2.23 (s, 3H), 6.25 (s, 1H), 6.96 (dd, ³J=8.1 Hz, ⁴J=2.1 Hz, 1H), 7.00 (s, 1H), 7.63 (d, ³J=8.1 Hz, 1H), 9.03 (s, 1H). ¹³C-NMR: 17.9, 20.4, 29.6, 34.3, 98.5, 121.6, 126.9, 129.5, 131.2, 132.5, 137.3, 161.5, 165.7. HRMS (ESI) Calculated m/z: 261.1420; found m/z: 261.1420 [M+H]⁺. Anal. Calcd for C₁₅H₂₀N₂S: C, 69.19; H, 7.74; N, 10.76. Found: C, 68.80; H, 7.53; N, 10.48.

### 4-(^{tert}-Butyl)-N-(2,3)dimethylphenyl)thiazol-2-amine (423, O4I4)

¹H-NMR: 1.23 (s, 9H), 2.13 (s, 3H), 2.25 (s, 3H), 6.26 (s, 1H), 6.93 (d, ³J=7.4 Hz, 1H), 7.05 (t, ³J=7.8 Hz, 1H), 7.52 (d, ³J=8.0 Hz, 1H), 9.13 (s, 1H). ¹³C NMR: 13.8, 20.3, 29.6, 34.5, 120.1, 125.6, 125.7, 128.8, 131.2, 137.2, 139.7, 161.5, 166.1. HRMS (ESI) Calculated m/z: 261.1420; found m/z: 261.1420 [M+H]⁺. Anal. Calcd for C₁₅H₂₀N₂S: C, 69.19; H, 7.74; N, 10.76. Found: C, 68.81; H, 7.63; N, 10.49.

### Cell Culture

The generation of HEK-Oct4, NCCIT-Oct4 and NCCIT-Nanog reporter cell lines, as well as HFF-LTR7-EGFP, is known in the art (Cheng et al, J Med Chem 2015, 58 (12), 4976-83; Cheng et al, J Med Chem 2015, 58 (15), 5742-50; Wang et al, Nature 2014, 516 (7531), 405-9).

Human primary fibroblasts were isolated as described elsewhere (approved by the Ethikkommission I Heidelberg S-186/2016) and cultivated in DMEM (Gibco, Germany) containing 10% FBS (Gibco) and 1% Penicillin/Streptomycin (Gibco). NCCIT was in RPM11640 (Gibco) containing 10% FBS and 1% P/S. Cells were maintained under 5% CO₂ at 37° C. in a humidified atmosphere. iPSCs were in Essential®8 medium (life technologies, Germany). Colony expansion was used for iPSCs maintenance, in which iPSC-like colonies were mechanically selected and isolated in a plate coated with Geltrex® (life technologies); the medium was refreshed very day.

### Luciferase Reporter Assay

Luciferase reporter assay was performed using Beatle Juice Kit (PJK, Germany) according to the manufacturer's instruction. NCCIT-Oct and NCCIT-Nanog reporter cells were plated into a 24-well plate (100,000 cells/well) for 24 h. Cells were treated with compounds as indicated and harvested with luciferase lysis buffer (25 mM Tris phosphate buffer pH=7.8, 4 mM EGTA, 1% Triton X-100, 10% glycerol and 2 mM fresh DTT, filtered through 0.45 μm sterile filter) at 37° C. for 15 min. The protein concentration was determined by Bradford assay (Sigma, Germany). 100 μL reaction mixture containing luciferin and ATP was added to 20 μL cell lysis in a white plate (Gibco, Germany), incubated for 5 min and measured by a luminometer plate-reader. The activity was determined as percent luminous intensity of treated cells over control cells from at least five-independent experiments.

qRT-PCR

Quantitative reverse-transcription real-time-PCR was performed using a Light Cycler 96 (Roche, Germany) following the manufacturer's protocol. Briefly, total RNA was isolated from cells using TRIzol (Qiagen, Germany) or NucleoSpin®RNA Plus (Macherey-Nagel, Germany). The same amount of RNA was used to reverse-transcriptional synthesize cDNA by using random primers ProtoScript® First Strand cDNA Synthesis Kit (NEB, Germany). qPCR was performed using the SYBR Green PCR master mix (qPCRBIO SyGreen Mix Lo-Rox, Nippon Genetics, Germany) and the primer pairs indicated below (MWG, Eurofins, Germany). Actin was used as an endogenous control. Data were normalized to the value of untreated cells showing the mean±SD of quadruplicates and are representative of at least three independent experiments.

TABLE 2
PCR primers

| Gene | Forward | Reverse |
|---|---|---|
| Oct 4 | SEQ ID 001: | SEQ ID 002: |
| | gaaggatgtggtccga | gtgaagtgagggctcc |
| | gtgt | cata |
| Sox2 | SEQ ID 003: | SEQ ID 004: |
| | aaccccaagatgcaca | gcttagcctcgtcgat |
| | actc | gaac |
| Nanog | SEQ ID 005: | SEQ ID 006: |
| | aatacctcagcctccag | tgcgtcacaccattgct |
| | cagatg | attcttc |
| Lin28A | SEQ ID 007: | SEQ ID 008: |
| | gtggatgtctttgtgca | gacacggatggattcc |
| | ccag | agac |
| Klf4 | SEQ ID 009: | SEQ ID 010: |
| | ggacctggactttattc | gataggtgaagctgc |
| | tctcc | aggtg |
| Esrrb | SEQ ID 011: | SEQ ID 012: |
| | ccttccaatcagctgc | gatctgcctgcctctc |
| | cttc | tcat |
| Nodal | SEQ ID 013: | SEQ ID 014: |
| | gtcgacatcacttgcc | tggtcggatgaaactc |
| | agac | ctcc |
| FGF2 | SEQ ID 015: | SEQ ID 016: |
| | aggagtgtgtgctaacc | cagttcgtttcagtgcc |
| | gtt | aca |
| E-Cadherin | SEQ ID 017: | SEQ ID 018: |
| | gagagactgggttattc | gatgctgtagaaaacctt |
| | ctc | gcc |
| Rex1 | SEQ ID 019: | SEQ ID 020: |
| | tggacacgtctgtgctc | gtcttggcgtcttctcg |
| | ttc | aac |
| Sall4 | SEQ ID 021: | SEQ ID 022: |
| | atttgtgggaccctcga | ggtaaaagctcgcccac |
| | cat | aaa |
| Actin | SEQ ID 023: | SEQ ID 024: |
| | ctgactacctcatgaag | cattgccaatggtgatg |
| | atcctc | acctg |

### Immunoblotting

iPSCs were lysed using urea-lysis buffer (1 mM EDTA, 0.5% Triton X-100, 5 mM NaF, 6 M Urea, 1 mM Na3VO4, 10 μg/mL Pepstatin, 100 μM PMSF and 3 μg/mL Aprotinin in PBS). Enhanced chemiluminescence (ECL) immunoblot analysis was performed. 40 μg of total protein was resolved on 12% SDS-PAGE gels and immunoblotted with antibodies. 13-Actin antibody was used as loading control. Primary antibodies were incubated at 1:1000 dilution in TBS (pH 7.5) with 0.1% Tween-20 and 5% BSA/milk with gentle agitation overnight at 4° C. Secondary antibodies (Dianova, Germany) were incubated in TBS (pH 7.5) with 5% Milk and 0.1% Tween-20 at a 1:10,000 dilution for 1 h at room temperature.

TABLE 3
Antibodies used

| Antibody | Company | Cat # |
|---|---|---|
| Oct4A | Cell | 2840S |
| (30A3) | signaling | |
| Sox2 | Cell | 4900 |
| | signaling | |
| Lin28 | epitomics | 3334-1 |
| E-Cadherin | Cell | 3195 |
| | Signaling | |
| Tra-1-81 | Cell | 4745P |
| | signaling | |
| β-Catenin | Cell | 8480P |
| (D10A8) XP | signaling | |
| β-Actin (C4) | santa | sc- |
| | cruz | 47778 |
| Nanog | Cell | 4893S |
| (1E6C4) | signaling | |
| Tra-1-60 (S) | Cell | 4746P |
| | signaling | |

### Immunocytochemistry

iPSCs were seeded in a 96-well plate coated with Geltrex. Cells were fixed with 4% PFA at RT for 15 min, and blocked with blocking buffer (5% goat serum and 0.3% Triton X-100 in PBS) for 1 h. Blocking solution was aspirated and incubated with antibodies in antibody dilution buffer (1% BSA and 0.2% Triton X-100 in PBS) at 4° C. overnight. The secondary antibodies (Goat anti-rabbit Alexa Flor 488 and Goat anti-mouse Alexa Flor 594, Dianova) were added and incubated for 1 h. Hoechst 33342 (1 μg/mL in PBS) was used to visualize nuclei.

### FACS

iPSCs were seeded in a 6-well plate coated with Geltrex. Cells were harvested, fixed and permeabilized (if appropriately) as known in the art (Cheng et al, Mol Oncol. 2016 June; 10(6):806-24). Fixed cells were incubated with the primary antibody for 24 h at 4° C. and with the secondary antibody for 1 h at room temperature.

### Sample Preparation for LC/MS Analysis

NCCIT cells were treated with O4I2 (50 μM) in RPMI 1640 medium containing 2% BSA/FBS. Medium and cells were collected. Cell pellet was solved in 0.1% Triton-x. Protein precipitated by adding MeOH (pH=2) in lysate supernatant on ice for 2 h. Metabolites were extracted from the combined supernatant with ethyl acetate. After removal of organic solvent, residue was dissolved in acetonitrile, frozen immediately in liquid N2 and stored in −80° C. until further analysis.

Additional methods including experimental procedures, chemical synthesis and characterization data can be found in the supplementary information.

### HPLC Analysis

Purity of synthetic products was verified using HPLC analysis on a Jasco HPLC system (PU 2085-plus, Jasco GmbH, Umstadt, Germany) equipped with a porous silica column (ReproSil-Pur ODS-3, dimensions 50×2 mm, Dr. Maisch GmbH, Ammerbuch-Entringen Germany). The synthetic products (10 mM in acetonitrile) were diluted to a final concentration of 25 μM in water. Solvent A consisted of water with 0.1% trifluoroacetic acid and solvent B consisted of acetonitrile with 0.1% trifluoroacetic acid. A linear gradient was applied going from 0% solvent B to 100% solvent B in 7 minutes, followed by another minute of 100% solvent B, after which the system was immediately brought back to 100% solvent A and flushed for 2 minutes. Absorbance was monitored at wavelength of 254 nm. It appeared that O4I2-COON eluted after approximately 3.65 minutes and O4I2 after approximately 4.50 minutes (see chromatograms in SI 3).

### LC-MS

Samples obtained after purification were diluted to contain 20% MeOH. 100 μL of the sample was transferred to an HPLC vial and placed in the autosampler of an Agilent 1200 series HPLC machine. Sample (20 μL) was injected into the system and metabolites were separated on a C18 column (Kinetex 2.6 μm, C18, 100 Å, dimensions 100×2.1 mm, Phenomenex, Aschaffenburg, Germany). Solvent A was water with 0.1% trifluoroacetic acid and solvent B was 80% acetonitrile in water with 0.1% trifluoroacetic acid. Metabolites were separated using a linear gradient from 20% solvent A to 80% solvent B in 40 minutes. Effluent of the column was sprayed directly into the source of the ESI-Q-TOF (electrospray ionization quadrupole time-of-flight) mass spectrometer (micrOTOF, Bruker Daltonics, Bremen, Germany). Scanning mass range was set to m/z 100-1000.

### Episome-Mediated Reprogramming

The episomal vectors carrying OCT44 (#41813), SOX2/KLF4 (#27078) and L-MYC/LIN28 (#27080) were purchased from Addgene (USA). To reach the maximal efficiency, episomal expression of EBNA1 (#37624) was co-transfected with OSKML. Reprogramming was performed in 24-well plate as previously reported (Okita et al. Nat Protoc 5, 418-428 (2010); Okita et al. Nat Methods 8, 409-412 (2011); Okita et al. Science 322, 949-953 (2008)). Briefly, 10 000-20 000 cells were seeded in a 24-well plate for 24 h and then treated with O4I3 (0.25 μM) for 48 h. Cells were transfected with OSKML or SKML in presence of O4I4 (5 μM for HF1 and HF2, 20 μM for HF3) for 7 days (HF1 and HF2) or for 5 days (HF3) in DMEM/F12 GlutaMAX knockout medium containing 1% N2, 2% B27 and 100 ng/mL bFGF. Lipofectamine 3000 was used as transfection reagent (Life Technologies). Essential®8 medium was used after 10 days. After 21-28 days, TRA-1-60 staining was conducted as described before (Cheng et al. J Med Chem 58, 4976-4983 (2015); Cheng et al. J Med Chem 58, 5742-5750 (2015)). TRA-1-60 positive iPSCs-like colonies were mechanically and manually isolated and transferred into a 6-well plate coated with Geltrex® or Matrigel in Essential®8 medium. Medium was refreshed daily.

Differentiation of Mono-Layer CSKML-iPSCs into Three Germ Layers

Mono layer iPSCs (density 60-80%) in Geltrex-coated 24-well plate were used for differentiation. Differentiation medium (500 mL) contains 5 mL BSA, 20 μL 1-thioglycerol, 10 μg/mL insulin, 15 μg/mL transferrin, 0.1% polyvinyl alcohol in DMEM/F12 Glutamax knockout medium.

Endoderm: Day 1) E8 medium was switched to differentiation medium (DE) additionally containing 100 ng/mL Activin, 100 ng/mL bFGF, 10 ng/mL BMP4, 10 μM Ly294002 and 2.5 μM Chir99021. Day 2) Medium was changed with DE containing 100 ng/mL Activin, 100 ng/mL bFGF, 10 ng/mL BMP4 and 10 μM Ly294002. Day 3) RPMI 1640 medium containing 100 ng/mL Activin and 100 ng/mL bFGF was used.

Mesoderm: E8 medium was switched to differentiation medium (DE) additionally containing 100 ng/mL Activin, 20 ng/mL bFGF, 10 ng/mL BMP4, 10 μM Ly294002 and 5 μM Chir99021. The medium was changed daily for 2-3 days.

Neuroectoderm: E8 medium was switched to differentiation medium (DE) additionally containing 10 μM SB431542, 1 μM LDN193189 and 10 ng/mL bFGF. The medium was changed daily for 14 days.

### In Vitro Differentiation of CSKML-iPSCs Via Embryoid Body

To spontaneously differentiate the novel human iPSCS to embryoid bodies (EBs), the differentiation was performed according to a previously published protocol (Wang et al. Nature 516, 405-409 (2014)). Briefly, the novel human iPSCs were cultured on matrigel-coated 6-well plates. Cells from one well were dissociated with collagenase IV (1 mg ml⁻¹), and then split into small cell clumps. The small cell clumps were transferred into one 10-cm low-attachment dish, and cultured in EB medium (knockout DMEM, 20% knockout serum replacement, 1 mM L-glutamine, 1% nonessential amino acids, 0.1 mM 2-mercaptoethanol and primocin). The medium was changed every 2 days. The EBs were cultured for 8-10 days followed by collection for RNA isolation and qPCR.

| Gene | Forward | Reverse |
|---|---|---|
| DNMT3B | SEQ ID NO 25: | SEQ ID NO 26: |
| | AGAGGGACATCTCACGGTTC | GGTTGCCCCAGAAGTATCG |
| DPPA2 | SEQ ID NO 27: | SEQ ID NO 28: |
| | GGTGCCAGTTAAAGATGACGC | GAGGCAAAATGGTCGGCAAG |
| DPPA4 | SEQ ID NO 29: | SEQ ID NO 30: |
| | GACCTCCACAGAGAAGT | TGCCTTTTTCTTAGGGCAGAG |
| | CGAG | |
| FGF4 | SEQ ID NO 31: | SEQ ID NO 32: |
| | GCAAGGGCAAGCTCTATGG | AGGACTCGTAGGCGTTGTA |
| | | GTT |
| GDF3 | SEQ ID NO 33: | SEQ ID NO 34: |
| | CGCTTTCTCCCAGACCAA | GGCAGACAGGTTAAAGTAGA |
| | | GGAG |
| MYC | SEQ ID NO 35: | SEQ ID NO 36: |
| | CACCAGCAGCGACTCTGA | GATCCAGACTCTGACCTTT |
| | | TGC |
| NANOG | SEQ ID NO 37: | SEQ ID NO 38: |
| | AGATGCCTCACACGGAGACT | GGACTGGTGGAAGAATCAGG |
| POU5F1 | SEQ ID NO 39: | SEQ ID NO 40: |
| | CGACCATCTGCCGCTTTG | GCCGCAGCTTACACATGTTCT |
| SALL4 | SEQ ID NO 41: | SEQ ID NO 42: |
| | AGCACATCAACTCGGAGGAG | CATTCCCTGGGTGGTTCACTG |
| SOX2 | SEQ ID NO 43: | SEQ ID NO 44: |
| | ACAGCAAATGACAGCTGC | TCGGCATCGCGGTTTTT |
| | AAA | |
| PAX6 | SEQ ID NO 45: | SEQ ID NO 46: |
| | GGTTGGTATCCGGGGACTTC | TCCGTTGGAACTGATGGAGT |
| SOX1 | SEQ ID NO 47: | SEQ ID NO 48: |
| | ACCAGGCCATGGATGAAG | CTTAATTGCTGGGGAATTGG |
| ZIC1 | SEQ ID NO 49: | SEQ ID NO 50: |
| | CTGGCTGTGGCAAGGTCTTC | CAGCCCTCAAACTCGCACTT |
| GATA4 | SEQ ID NO 51: | SEQ ID NO 52: |
| | GGAAGCCCAAGAACCTGAAT | GTTGCTGGAGTTGCTGGAA |
| GATA6 | SEQ ID NO 53: | SEQ ID NO 54: |
| | AATACTTCCCCCACAACA | CTCTCCCGCACCAGTCAT |
| | CAA | |
| SOX17 | SEQ ID NO 55: | SEQ ID NO 56: |
| | ACGCCGAGTTGAGCAAGA | TCTGCCTCCTCCACGAAG |
| EOMES | SEQ ID NO 57: | SEQ ID NO 58: |
| | ACCGCCACCAAACTGAGAT | TTGTAGTGGGCAGTGGGATT |
| HAND1 | SEQ ID NO 59: | SEQ ID NO 60: |
| | AACTCAAGAAGGCGGATGG | GGAGGAAAACCTTCGTGCT |
| T | SEQ ID NO 61: | SEQ ID NO 62: |
| | ACCCAGTTCATAGCGGTGAC | CCATTGGGAGTACCCAGGTT |
| GAPDH | SEQ ID NO 63: | SEQ ID NO 64: |
| | agccacatcgctcagacac | gcccaatacgaccaaatcc |

### Bisulfite Sequencing

The bisulfite reaction was performed using the EpiTect Bisulfite Kit (Qiagen) according to the manufacturer's instructions. 5 ul of bisulfite-treated genomic DNA was used to amplify the OCT4 a promoter region in human iPSCs and the parental fibroblast. PCR products were cloned into pGEM-T vectors (Promega) and sequenced using the M13 forward primer. Primers used to amplify the OCT4 promoter region were described in a previous publication (Grabundzija et al. Nucleic acids research 41, 1829-1847 (2013)).

| | | |
|---|---|---|
| | SEQ ID NO 65: | SEQ ID NO 66: |
| | GGATGTTATTAAGAT | CCTAAACTCCCCTT |
| OCT4_meth | GAAGATAGTTGG | CAAAATCTATT |

### ATAC-Seq

Cells (50 000) were lysed in a lysis buffer (10 mM Tris HCL pH 7.4, 10 mM NaCl, 3 mM MgCl₂, 0.1% IGEPAL-CA630). TransNGS®Tn5 DNA library pre Kit for Illumina was used to prepare DNA library for sequencing.

### Reprogramming

Epi5® Episomal iPSC Reprogramming Kit (Life technologies) was used to reprogram human fibroblasts according to the manufacturer's protocol. Briefly, 5,000-50,000 cells were seeded in a 24-well plate for 24 h and then transfected with episomal vectors in N2 B27 medium. After 14 days, cells were re-suspended with 0.5 mM EDTA and re-plated in a 6-well plate in Essential®8 medium. Colonies were isolated after 3-4 weeks with the help of alkaline phosphatase (AP) live stain (Life technologies). Medium was refreshed daily.

iPSC Single Cell Expansion

iPSC single cell expansion was performed according to a well-established protocol known in the art (Watanabe et al, Nat Biotechnol, 2007, 25(6), 681-6). Briefly, iPSCs were dissociated with Accutase® as single cell suspension and re-seeded in a Geltrex-coated 96-well plate in the presence of Rock inhibitor for the first 24 h in combination with/without O4I3. Cell viability was performed in MTT assay as disclosed elsewhere (Cheng et al, Mol Oncol. 2016 June; 10(6):806-24).

### Gene Expression Profiling and Data Analysis

Gene expression profiling of human PSCs, iPSCs and fibroblast samples was conducted on the Illumina Human Sentrix-12v4 BeadChip array by the Genomics and Proteomics Core Facility of DKFZ. Two sets of samples were profiled: (i) three hPSC samples, corresponding to treatments with two O4I3 doses (1 nM and 10 nM) and not-treated hPSCs; (ii) three fibroblast samples, corresponding to two treatments (one with O4I3 and one with OSKM) and not-treated fibroblast.

Gene expression analysis was performed in R 3.3.2 computing environment and packages from the open-source software development project Bioconductor 3.4. Clustering and functional enrichment analysis was performed with the standalone tool STEM 1.3.11, while heatmap visualization of gene expression was performed with the package pheatmap 1.0.8. The genome-wide expression profiles of iPSCs were determined using the Human-HT12-V4 Expression BeadChip. Three samples were profiled, corresponding to two O4I3 treatments (one with 1 μM and one with 10 μM) and not-treated iPSCs. Quality assessment and pre-processing of raw data was done with the package beadarray 2.24.2. Pre-processing involved default image processing (with median-based local background), default Illumina removal for outlying observations, mean summarization of bead-level observations into probe-level data, quantile normalization of probe-level data and log 2 transformation. The resulting probes were matched to genes using annotations from the package illuminaHumanv4.db 1.26.0. After filtering probes that poorly matched the annotated genes (quality status of no-match and bad) as well as low-expression probes (detection score>0.05), 14109 probes were available for clustering analysis with STEM, a tool for clustering short time series data that can differentiate between real and random temporal expression patterns. For this purpose, one probe per gene (the one with highest variance, if multiple probes matched to single gene) was selected and the data converted into fold changes with respect to the not-treated iPSCs. The data was analyzed as short time series of length three (corresponding to dose level of 0, 1, and 10 μM), by treating dose level as time factor. STEM was applied in default mode, except for the following settings: at least one measurement with absolute fold change of 0.65; significance of model profiles corrected by false discovery rate method; cellular component terms, terms with evidence code IEA and NAS or terms that had less than 10 genes in-common with the identified model profiles (or clusters of similar model profiles) were excluded from the Gene Ontology (GO) enrichment analysis; multiple testing correction of GO term significance values were determined by the randomization test, with 5000 randomly sampled gene sets per term.

Fibroblast/iPSC experiment data analysis; approximately 18 000 probes, after filtering, were available for differentially expression analysis. The latter was based on fold changes, i.e., the probes with at least two-fold expression relative to the untreated fibroblast were selected as differentially expressed; since single biological replicates per treatment were used, statistical tests were not applicable. Global gene expression comparison between pairs of samples were visualized by scatter plots, plotted with the package ggplot2 2.2.1, while the heatmaps of gene expressions were visualized with the package ComplexHeatmap 1.12.0. In addition, columns and rows in the heatmaps were reordered with hierarchical clustering based on average linkage and Euclidean distance or Pearson correlation. Functional enrichment analysis on differentially expressed probes was performed with gProfileR version r1732_e89_eg36 via the R interface in the package gProfileR version 0.6.1. More specifically, the query lists were treated as unordered human gene lists, and the set of probes retained after filtering was supplied as a background. Multiple testing correction was done by the FDR method according to Benjamini and Hochberg with 0.05 FDR cut-off, including biological processes and molecular functions from the Gene Ontology database with size of 5 up to 500 genes.

### Analysis of H₃K4Me3 Activation

Fibroblasts were treated with O4I3 0.5 μM for 2 days in the presence/absence of reprogramming transcription factors. The expression of H3K4Me3 was visualized by using H3K4Me3 antibody as described before for immunocytochemistry. The signal intensity was analyzed using ImageJ software as single cell level randomly selected from at least three independent experiments.

### Statistics

Statistical and graphical analyses of the data (except DNA microarray) were performed using Microsoft Excel 2007 software. The statistical significance of compared measurements was performed by using the Student's one-tailed or two-tailed t-test, considering P=0.05 as statistically significant.

### TRA-1-60+ Colonies Counting

To assess the number of TRA-1-60+ colonies, 10,000 fibroblast cells/well in 96-well plates were transfected with episomal OSKM (Epi5 kit, Thermofisher Scientific) in the presence/absence of small molecules, as indicated in the text. Immunostaining was performed using TRA-1-60 antibody in living cells after 32 days. Transfection efficiency was indicated by quantifying the expression of EGFP (Addgene plasmid #27082).

iPSCs Differentiation

Cellartis iPS cell to hepatocyte differentiation system (Takara, Germany) was used for differentiation of iPSCs to endoderm. StemDiff neuron differentiation kit (08500, Stem Cell Technologies, Germany) was used for differentiation of iPSCs to neuroectoderm. To differentiate iPSCs to mesoderm, cells were seeded in Geltrex-coated plates as single cells and cultivated till the density reached to at least 80%. N2+627 medium was used to replace E8 medium. Activin (100 ng/mL), bFGF (20 ng/mL), BMP4 (10 ng/mL), Ly2 (10 μM) and Chir (5μ) were freshly added. The medium was changed every two days for 14 days.

Measurement of methylated H₃K4 and the in vitro activity of LSD1, KDM4, and KDM5 Demethylases

Epiquik histone demethylase (H3K4 specific) activity/inhibition assay kit (Epigentek, Biocat, Germany) was used to measure the protective effect of O4I3 and zolpidem on the methylation of H3K4. Their inhibitory effects on LSD1, KDM5s, and KDM4 were determined using Epigenase jarid demethylase activity/inhibition assay kit, Epigenase JMJD2 demethylase activity/inhibition assay kit and EpiQuik histone demethylase LSD1 activity/inhibition assay kit (Epigentek, Biocat, Germany). The total nuclear extraction was used according to manufacturer's instructions. For measuring KDM5A, KDM5B, KDM5C, and KDM5D activity, magnetic beads were used to immunoprecipitate the respective enzymes from 10⁸ cells according to the manufacturer's instructions (Cell Signaling Technologies).

### Chromatin Immunoprecipitation Assay

SimpleChIP Plus Enzymatic Chromatin IP Kit (Cell Signaling Technologies) was used to isolate DNA precipitated with H3K4Me3 or H3K27Me3 antibody. According to the manufacturer's protocol, 10⁶ cells/sample were collected. Non-target Rabbit IgG and total Histone H3 antibodies (provided in the kit) were used as negative and positive controls, respectively. KDM5A-D enzymes were purified using immunoprecipitation with magnetic beads according to the manufacture's protocol. Recruited DNA was subjected to qRT-PCR using ChIP primers sets, and human RPL30 exon 3 primer pairs were used as positive control.

C. elegans Strains and Cultural Condition

Three strains N₂ (wild type), TJ356 [daf-16p::daf-16a/b::GFP+rol-6(su1006)] and CF1038 [daf-16(mu86)] were offered by Caenorhabditis Genetics Center (CGC, University of Minnesota, USA). All strains were maintained on nematode growth media (NGM) agar, which content Escherichia coli OP50 as a nutrient source and incubated at 20° C.

### Worm Synchronization

Eggs were released by lysing gravid adults with bleaching for 10 min. Resuspended eggs in sterile M9 buffer and incubated at 20° C. for 16-18 h. After all the worms were synchronized at the same L1 larval stage, transferred them into the liquid S-media which contents E. coli OP50 (0D600=0.8-1.2). Then, synchronized worms were treated with DMSO, 50 μg/ml epigallocatechin gallate (EGCG) or O4I4 (5 μM, 10 μM, and 20 μM) for further experiments.

### Intracellular ROS

2′-7′-dichlorofluorescein diacetate (H₂DCFDA) is a broadly known substance to quantify the ROS (reactive oxygen species) in C. elegans. After H₂DCFDA (non-fluorescent) penetrates the cell membrane, it will be turned into a highly fluorescent dye 2′-7-dichlorofluorescein (DCF) via the biochemical reaction of enzymatically deacetylated and oxidization by ROS. Due to the fluorescent intensity, the ROS level in C. elegans can be estimated.

The strains N2 (wild type) and CF1038 (daf-16 mutant) were synchronized and treated as described before. 50 μM H₂DCFDA was added into all samples after incubated 48 h. Samples were covered by foil paper to avoid the light and incubated another 1 h. Afterward, 10 μl worms were transferred on a glass slide and paralyzed with 10 μl of 10 mM sodium azide. BIOREVO BZ9000 fluorescence microscope was used to detect the production of DCF, and Image J 1.52a could analyze the ROS level in C. elegans via estimating the densitometrically.

### Survival Assay

After 48 h treatment, around 60 wild-type C. elegans (N2) worms in each sample were transferred into a new liquid S-media which contains 80 μM of superoxide-generating compound juglone (5-hydroxy-1,4-naphthalenedione) for 24 h. If a worm has response via a gentle touch with platinum wire, it is considered as a live worm. Conversely, a dead worm was considered by no response. The function of the survival rate is shown below.

$$\frac{live worms}{total worms} \times 100 \% = Survival Rate ( \% )$$

### Daf-16 Localization Assay

In this experiment, transgenic worms TJ356 (Daf-16::GFP) are employed to study our target substance affects the localization of daf-1 6. After 24 h treatment, worms were transferred to a glass slide and paralyzed with 10 mM sodium azide. BZ9000 fluorescence microscope was used to observe the localization of daf-1 6. In each sample, around 50 worms were scored for three localization patterns: cytoplasmic, intermediate and nuclear.

### Lifespan Assay

O4I4 (0.5 μM) were continuously added in medium until all the worms die. During the experiment period, every two days or three days, worms were counted either live or die by responding to a gentle touch with a platinum wire. Dead worms were discarded, and live worms were transferred to a new NGM plate to ensure they do not go through the dauer stage.

We claim:

1. A method of producing a pluripotent stem cell, in particular an induced pluripotent stem cell, said method comprising contacting a non-pluripotent donor cell obtained from a mammalian (particularly a human) donor with a compound characterized by general formula (1), 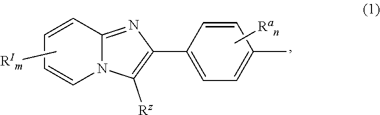
wherein
m is 1 or 2,
each R^{I}, independently from any other R^{I}, is selected from —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃,
R^{z} is selected from: 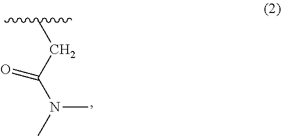 [SMILES: *CC(=O)N(C)C]
or —H,
n is 1 or 2,
each R^{a} independently from any other R^{a} is selected from —H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃,
or
two R^{a} on adjacent positions are linked by C₄H₄.

2. The method according to claim 1, wherein the compound is characterized by general formula (3), 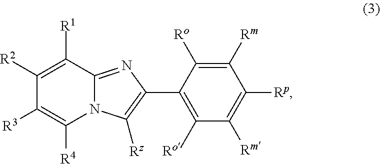
wherein
one of R¹, R², R³ and R⁴ is selected from:
—H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃,
and the other ones are —H.
R^{z} is selected from general formula (2) or —H,
one of R^{o}, R^{m}, R^{p}, R^{m′} and R^{o′} is selected from:
H, substituted or unsubstituted C₁-C₃ alkyl, substituted or unsubstituted C₁-C₃ alkoxy, —F, —Cl, —Br, —I, —NO₂, —OH, —NH₂, —SO₃H, —CN, substituted or unsubstituted C₁-C₃ carboxylic acid, —NCO, —CNO, —NCS, —SCN, —CH₂F, —CHF₂, or —CF₃,
and the other ones are
H, or
two neighbouring positions selected from R^{o}, R^{m} and R^{p} together are C₄H₄.

3. The method according to claim 1, wherein
each R^{I}, R^{a}, R¹, R², R³, R⁴, R^{o}, R^{m}, R^{p}, R^{m′} and R^{o′} is independently selected from —H, —CH₃, —F, —Cl, —Br, —I, —OH, —NO₂, —CN or —OMe.

4. The method according to claim 1, wherein
R¹ is selected from —CH₃, and —H;
R² is selected from —CH₃, and —H;
R³ is selected from —CH₃, —H, —F, —Cl, and —Br;
R⁴ is selected from —CH₃, and —H;
R^{o} and R^{o′} are selected from —NO₂, —F, and —H;
R^{p} is selected from —CH₃, —F, —Cl, —Br, —NO₂, —OMe, —OH, —CN, and —H;
R^{m} and R^{m′} are selected from —Cl, —H, —F, —OH, —NO₂, and —OMe, and/or
R^{z} is selected from —H and formula (2).

5. The method according to claim 1, wherein the compound according to formula (1) is
a) N,N-dimethyl-2-(6-methyl-2-p-tolylimidazo[1,2-a]pyridine-3-yl)acetamid (100),
b) 6-methyl-2-(p-tolyl)imidazo[1,2-a]pyridine (101),
c) 2-(4-fluorophenyl)-6-methyl-imidazo[1,2-a]pyridine (102),
d) 2-(4-chlorophenyl)-6-methyl-imidazo[1,2-a]pyridine (103),
e) 2-(4-bromophenyl)-6-methyl-imidazo[1,2-a]pyridine (104),
f) 6-methyl-2-(4-nitrophenyl)imidazo[1,2-a]pyridine (105),
g) 2-(4-methoxyphenyl)-6-methyl-imidazo[1,2-a]pyridine (106),
h) 4-(6-methylimidazo[1,2-a]pyridin-2-yl)phenol (107),
i) 4-(6-methylimidazo[1,2-a]pyridin-2-yl)benzonitrile (108),
j) 6-methyl-2-phenyl-imidazo[1,2-a]pyridine (109),
k) 6-methyl-2-(3-nitrophenyl)imidazo[1,2-a]pyridine (110),
l) 6-methyl-2-(2-nitrophenyl)imidazo[1,2-a]pyridine (111),
m) 2-(3-fluorophenyl)-6-methyl-imidazo[1,2-a]pyridine (112),
n) 2-(2-fluorophenyl)-6-methyl-imidazo[1,2-a]pyridine (113),
o) 2-(3-chlorophenyl)-6-methyl-imidazo[1,2-a]pyridine (114),
p) 3-(6-methylimidazo[1,2-a]pyridin-2-yl)phenol (115),
q) 2-(3-methoxyphenyl)-6-methyl-imidazo[1,2-a]pyridine (116),
r) 7-methyl-2-(p-tolyl)imidazo[1,2-a]pyridine (117),
s) 8-methyl-2-(p-tolyl)imidazo[1,2-a]pyridine (118),
t) 5-methyl-2-(p-tolyl)imidazo[1,2-a]pyridine (119),
u) 6-chloro-2-(p-tolyl)imidazo[1,2-a]pyridine (120),
v) 6-fluoro-2-(p-tolyl)imidazo[1,2-a]pyridine (121),
w) 6-bromo-2-(p-tolyl)imidazo[1,2-a]pyridine (122), or
x) 6-methyl-2-(2-naphthyl)imidazo[1,2-a]pyridine (123).

6. A method of producing a pluripotent stem cell, in particular a method of producing an induced pluripotent stem cell, said method comprising contacting a non-pluripotent donor cell obtained from a mammalian (particularly a human) donor with a compound characterized by general formula (4), 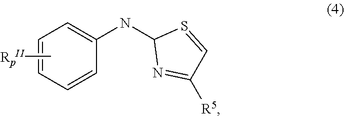
wherein
each R^{II}, independently from any other R^{II}, is selected from
—H,
substituted or unsubstituted C₁-C₃ alkyl, particularly methyl,
substituted or unsubstituted C₁-C₃ alkoxy, particularly O—CH₃,
—F, —Cl, —Br, —I, —NO₂, —OH, —NH₂, —SO₃H, —CN, —NCO, —CNO, —NCS, —SCN, particularly —F, —Cl, —Br, —NO₂, —OH, and —CN,
substituted or unsubstituted C₁-C₃ carboxylic acid, —CH₂F, —CHF₂, or —CF₃, particularly COOH;
p is 1, 2 or 3, particularly p is 1 or 2;
R⁵ is selected from
—H,
substituted or unsubstituted C₁-C₄ alkyl, particularly t-butyl,
substituted or unsubstituted aryl, particularly unsubstituted phenyl or naphtyl;
COOR, wherein R is an unsubstituted or substituted C₁-C₄ alkyl, particularly R is an unsubstituted C₁-C₄ alkyl, more particularly R is ethyl;
with the proviso that the following combinations are not encompassed in the definition of compound (4):
R⁵ is COOR with R being an unsubstituted or substituted C₁-C₄ alkyl, particularly R is an unsubstituted C₁-C₄ alkyl, more particularly R is ethyl, and
R^{II} is halogen or hydrogen and p is selected from 1 or 2.

7. The method according to claim 6, wherein the compound according to formula (4) is the compound according to formula (5), (6) or (7): 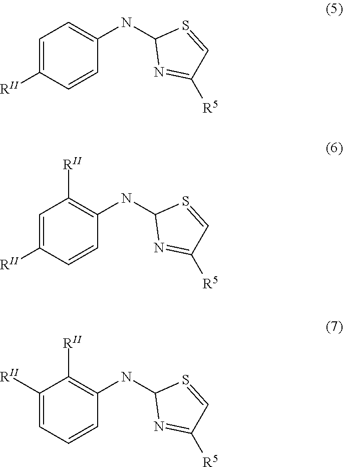
wherein R^{II} and R⁵ have the same meanings as indicated in claim 6.

8. The method according to claim 6, wherein each R^{II}, independently from any other R^{II}, is selected from methyl and ethyl, particularly all R^{II} are methyl.

9. The method according to claim 6, wherein R⁵ is selected from
substituted or unsubstituted C₃-C₄ alkyl, particularly unsubstituted t-butyl, and
substituted or unsubstituted aryl, particularly unsubstituted phenyl or naphtyl.

10. The method according to claim 6, wherein the compound according to formula (4) is selected from any one of the formulas (419), (420), (421), (422) and (423), particularly wherein the compound according to formula (4) is (423)

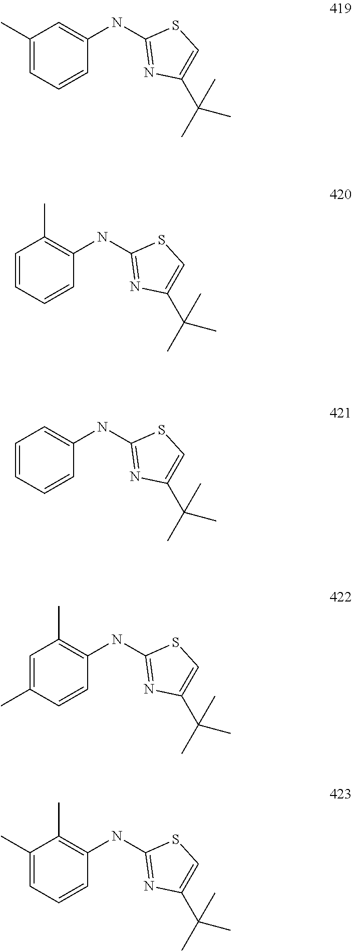

11. The method according to claim 6, wherein the method further comprises introducing into said mammalian non-pluripotent donor cell at least one element selected from:
a nucleic acid sequence encoding a polypeptide,
a polypeptide,
a non-coding nucleic acid sequence,
wherein said polypeptide is selected from:
the family of OCT polypeptides, in particular OCT4,
the family of KLF polypeptides, in particular KLF4,
the family of SOX polypeptides, in particular SOX2,
the family of MYC polypeptides, in particular L-MYC, and
the family of LIN polypeptides, in particular Lin28.
and wherein said non-coding nucleic acid sequence is selected from miR-302b, miR-372, miR-302, miR-367, miR-200c and miR-369.

12. A method of inducing OCT4 and/or NANOG in a mammalian cell, or a method of increasing methylation of histone 3 lysine 4 (H₃K4) in a mammalian non-pluripotent donor cell to augment a method of producing an induced pluripotent stem cell, said method comprising contacting a mammalian donor cell with a compound as specified in claim 1.

13. A method of increasing methylation of histone 3 lysine 4 (H₃K4) in a mammalian non-pluripotent donor cell to augment a method of producing an induced pluripotent stem cell, comprising contacting said donor cell with a compound as specified in claim 1.

14. A method of maintaining pluripotency in mammalian pluripotent stem cells comprising contacting said mammalian pluripotent stem cell with a compound as specified in claim 1.

15. The method of claim 1, wherein the method is practiced in vitro or ex vivo.

16. The method according to claim 1, wherein said compound is used in a final concentration between 0.0005 μmol/l and 100 μmol/l, in particular between 0.001 μmol/l and 15 μmol/l, more particular between 0.001 μmol/l and 10 μmol/l.

17. An induced pluripotent mammalian stem cell obtained or obtainable by a method according to claim 1.

18. A cell culture medium comprising a compound as specified in claim 1.

19. The cell culture medium according to claim 18, wherein said compound has a defined final concentration between 0.5 nmol/l and 100 μmol/l, in particular between 1 nmol/l and 15 μmol/l, more particular between 1 nmol/l and 10 μmol/l.

20. A composition for promoting formation of induced pluripotent mammalian stem cells, comprising a compound specified in claim 1.
